(19) United States
(12) Patent Application Publication
(10) Pub. No.: US 2026/0265380 A1
(43) Pub. Date: Sep. 10, 2026

(54) COMBINATION OF AN ATP-HYDROLYZING ENZYME AND AN IMMUNE CHECKPOINT MODULATOR AND USES THEREOF

(71) Applicants: MV BIOTHERAPEUTICS SA, Bellinzona (CH); INSTITUTE FOR RESEARCH IN BIOMEDICINE, Bellinzona (CH)

(72) Inventors: Fabio GRASSI, Bellinzona (CH); Benedetta DE PONTE CONTI, Milan (IT)

(21) Appl. No.: 19/685,509

(22) Filed: May 22, 2026

Related U.S. Application Data
(63) Continuation of application No. 17/999,455, filed on Jun. 1, 2021.

(30) Foreign Application Priority Data
Jun. 3, 2020 (WO) PCT/EP2020/065357

(51) Int. Cl. C07K 16/28; A61K 9/00; A61K 39/00; A61P 35/00; C12N 9/14; C12N 15/70; C12N 15/74

(52) U.S. Cl. CPC C07K 16/2827; A61K 9/0053; A61K 39/0011; A61P 35/00; C12N 9/14; C12N 15/70; C12N 15/74; A61K 2039/5156; A61K 2039/5158; A61K 2039/55516; C07K 2317/76

(57) ABSTRACT

The present invention provides a combination of (i) an immune checkpoint modulator and (ii) an ATP hydrolyzing enzyme, a nucleic acid encoding an ATP hydrolyzing enzyme, or host cells, microorganisms or viral particles comprising such nucleic acids encoding an ATP hydrolyzing enzyme. The combination may be used in medicine, in particular in the treatment of cancer, for example in cancer immunotherapy.

### CROSS-REFERENCE TO RELATED APPLICATIONS

This patent application is a continuation application of U.S. application Ser. No. 17/999,455, filed Nov. 21, 2022, which is a national stage application of International Application No. PCT/EP2021/064659, filed Jun. 1, 2021, which claims the benefit of International Application No. PCT/EP2020/065357, filed Jun. 3, 2020, each of which is incorporated herein by reference in its entirety for all purposes.

### REFERENCE TO SEQUENCE LISTING

The instant application contains a Sequence Listing which has been submitted electronically in XML file format and is hereby incorporated by reference in its entirety. Said XML copy, created on May 18, 2026 is named 0215-MV01_SL.xml and is 18,402 bytes in size.

### FIELD

The present invention relates to the field of immunotherapy by modulation of immune checkpoints, for example in cancer immunotherapy. The present invention provides new combinations and methods to improve immunotherapy by modulation of immune checkpoints, for example in cancer immunotherapy. In particular, the present invention provides the combination of an immune checkpoint modulator with an ATP-hydrolyzing enzyme, a nucleic acid encoding an ATP-hydrolyzing enzyme, or a host cell, a microorganism or a viral particle comprising a nucleic acid encoding an ATP-hydrolyzing enzyme and uses thereof.

### BACKGROUND

Cancer immunotherapy with immune checkpoint inhibitors increases antitumor immunity by blocking inhibitory immune checkpoints. Inhibitory immune checkpoints typically prevent excessive immune responses. Thereby, immune checkpoints may prevent the immune system, e.g. T cells, from effectively attacking cancer cells. In particular, cancer cells can activate different immune checkpoint pathways to exploit their immunosuppressive functions. Accordingly, blockade of immune checkpoints “unleashes” the immune system, such that immune responses against cancer cells are induced or enhanced. Prominent examples of checkpoint proteins found on T cells or cancer cells include programmed cell death protein 1 (PD-1)/programmed death-ligand 1 (PD-L1) or cytotoxic-T-lymphocyte-associated protein 4 (CTLA-4), which suppress T cell anti-tumor activity. In the recent years, PD-1/PD-L1 and CTLA-4 inhibitors showed promising therapeutic outcomes, and have been approved for various cancer treatments, while further immune checkpoint inhibitors are currently under investigation and in clinical trials.

Recently, extracellular adenosine was identified as potent immune checkpoint mediator interfering with antitumor immune responses. Immunosuppressive adenosine is generated by hydrolysis of adenosine triphosphate (ATP). In humans, the rate-limiting ecto-enzyme in ATP hydrolysis is CD39 and inhibition of ATP-hydrolyzing enzyme CD39 was recently suggested for cancer treatment (Allard B, Longhi M S, Robson S C, Stagg J. The ectonucleotidases CD39 and CD73: Novel checkpoint inhibitor targets. Immunol Rev. 2017; 276(1):121-144. doi:10.1111/imr.12528; Allard D, Allard B, Stagg J. On the mechanism of anti-CD39 immune checkpoint therapy. J Immunother Cancer. 2020; 8(1):e000186. doi:10.1136/jitc-2019-000186). It was demonstrated that CD39 expression by Tregs plays a permissive role in a mouse model of hepatic metastasis, developed through portal vein infusion of luciferase-expressing melanoma B16/F10 cells and MCA-38 colon cancer cells into wild type and CD39^{-/-} mice (Sun X, Wu Y, Gao W, et al. CD39/ENTPD1 expression by CD4+Foxp3+ regulatory T cells promotes hepatic metastatic tumor growth in mice. Gastroenterology. 2010; 139:1030-1040). Growth of melanoma metastatic tumors was strongly inhibited in CD39^{-/-} mice or in chimeric mice reconstituted with CD39^{-/-} bone marrow derived cells (Sun X, Wu Y, Gao W, et al. CD39/ENTPD1 expression by CD4+Foxp3+ regulatory T cells promotes hepatic metastatic tumor growth in mice. Gastroenterology. 2010; 139:1030-1040). Moreover, treatment with polyoxometalate 1 (POM1), a pharmacological CD39 inhibitor, was also shown to significantly limit the tumor growth (Kunzli B M, Bernlochner M I, Rath S, et al. Impact of CD39 and purinergic signalling on the growth and metastasis of colorectal cancer. Purinergic Signal. 2011; 7:231-241). Interestingly, synergistic effects of combination therapy associating CD39 inhibition with immune checkpoint blockade were recently reported. In lung metastasis models, specific blockade of CD39 with POM-1 significantly enhanced the antitumor activity of anti-PD1 and anti-CTLA-4 mAb, in an NK cell and IFN-γ dependent manner (Zhang H, Vijayan D, Li X-Y, et al. The role of NK cells and CD39 in the immunological control of tumor metastases. Oncoimmunology 2019; 8:e159380910.1080/2162402X.2019.1593809). Enhanced activity of anti-PD1 and anti-CTLA4 therapy in CD39-deficient mice inoculated with B16 or MCA205 tumors further suggest that targeted blockade of CD39 with anti-CD39 antibody IPH5201 (Innate Pharma) may also synergize with immune checkpoint inhibitors (Perrot I, Michaud H-A, Giraudon-Paoli M, et al. Blocking antibodies targeting the CD39/CD73 immunosuppressive pathway Unleash immune responses in combination cancer therapies. Cell Rep 2019; 27:2411-25.10.1016/j.celrep.2019.04.091).

While recent reports have shown that patients with various malignancies can benefit from immune checkpoint inhibitor treatment, therapeutic responses were observed only in a limited fraction of patients treated with immune checkpoint inhibitors (Schoenfeld, A. J., and Hellmann, M. D. (2020). Acquired Resistance to Immune Checkpoint Inhibitors. Cancer Cell 37, 443-455). Resistance to the therapeutic effect of immune checkpoint blockade (ICB) may be classified into two broad categories: (i) primary resistance, generally referring to patients who do not respond at all to ICB; and (ii) acquired resistance, which refers to patients showing an initial response to ICB followed by progression of disease. To antagonize primary resistance a number of combinatorial therapies (e.g. chemotherapy, tyrosine kinase and growth factor inhibitors) are currently under investigation (Gandhi, L., Rodriguez-Abreu, D., Gadgeel, S., Esteban, E., Felip, E., De Angelis, F., Domine, M., Clingan, P., Hochmair, M. J., Powell, S. F., et al. (2018). Pembrolizumab plus Chemotherapy in Metastatic Non-Small-Cell Lung Cancer. N Engl J Med 378, 2078-2092; Motzer, R. J., Penkov, K., Haanen, J., Rini, B., Albiges, L., Campbell, M. T., Venugopal, B., Kollmannsberger, C., Negrier, S., Uemura, M., et al. (2019). Avelumab plus Axitinib versus Sunitinib for Advanced Renal-Cell Carcinoma. N Engl J Med 380, 1103-1115; Schmid, P., Adams, S., Rugo, H. S., Schneeweiss, A., Barrios, C. H., Iwata, H., Dieras, V., Hegg, R., Im, S. A., Shaw Wright, G., et al. (2018). Atezolizumab and Nab-Paclitaxel in Advanced Triple-Negative Breast Cancer. N Engl J Med 379, 2108-2121). However, mechanisms leading to acquired resistance to ICB are unknown and there have been no therapeutic approaches for reversing it.

In immune checkpoint blockade (ICB) responsive patients, the tumor microenvironment (TME) was shown to be replenished with fresh, non-exhausted CD8⁺ T cells and T cell clones from sites outside the tumor. This phenomenon appears to be the key factor in explaining the clinical benefit from cancer immunotherapy (Wu, T. D., Madireddi, S., de Almeida, P. E., Banchereau, R., Chen, Y. J., Chitre, A. S., Chiang, E. Y., Iftikhar, H., O'Gorman, W. E., Au-Yeung, A., et al. (2020). Peripheral T cell expansion predicts tumour infiltration and clinical response. Nature 579, 274-278). In fact, antigen recognition by cytotoxic T cells within the TME favor the expansion of dysfunctional cells that become exhausted and epigenetically locked, thereby difficult to revert to effector functionalities (Khan, O., Giles, J. R., McDonald, S., Manne, S., Ngiow, S. F., Patel, K. P., Werner, M. T., Huang, A. C., Alexander, K. A., Wu, J. E., et al. (2019). TOX transcriptionally and epigenetically programs CD8. Nature 571, 211-218; Scott, A. C., DQndar, F., Zumbo, P., Chandran, S. S., Klebanoff, C. A., Shakiba, M., Trivedi, P., Menocal, L., Appleby, H., Camara, S., et al. (2019). TOX is a critical regulator of tumour-specific T cell differentiation. Nature 571, 270-274). Moreover, recent studies have shown that cytotoxicity is confined to non-tumor specific bystander cells that infiltrate the TME (Scheper, W., Kelderman, S., Fanchi, L. F., Linnemann, C., Bendle, G., de Rooij, M. A. J., Hirt, C., Mezzadra, R., Slagter, M., Dijkstra, K., et al. (2019). Low and variable tumor reactivity of the intratumoral TCR repertoire in human cancers. Nat Med 25, 89-94; Simoni, Y., Becht, E., Fehlings, M., Loh, C. Y., Koo, S. L., Teng, K. W. W., Yeong, J. P. S., Nahar, R., Zhang, T., Kared, H., et al. (2018). Bystander CD8(+) T cells are abundant and phenotypically distinct in human tumour infiltrates. Nature 557, 575-579).

Memory CD8+ T cells were shown to be recruited to the tumor irrespective of antigen specificity (Erkes, D. A., Smith, C. J., Wilski, N. A., Caldeira-Dantas, S., Mohgbeli, T., and Snyder, C. M. (2017). Virus-Specific CD8. J Immunol 198, 2979-2988; Rosato, P. C., Wijeyesinghe, S., Stolley, J. M., Nelson, C. E., Davis, R. L., Manlove, L. S., Pennell, C. A., Blazar, B. R., Chen, C. C., Geller, M. A., et al. (2019). Virus-specific memoryT cells populate tumors and can be repurposed for tumor immunotherapy. Nat Commun 10, 567; Simoni, Y., Becht, E., Fehlings, M., Loh, C. Y., Koo, S. L., Teng, K. W. W., Yeong, J. P. S., Nahar, R., Zhang, T., Kared, H., et al. (2018). Bystander CD8(+) T cells are abundant and phenotypically distinct in human tumour infiltrates. Nature 557, 575-579). These CD8⁺ tumor infiltrating lymphocytes (TILs) do not require cognate antigen recognition to become activated, perform effector functions, and improve host outcome (Martin, M. D., Jensen, I. J., Ishizuka, A. S., Lefebvre, M., Shan, Q., Xue, H. H., Harty, J. T., Seder, R. A., and Badovinac, V. P. (2019). Bystander responses impact accurate detection of murine and human antigen-specific CD8 T cells. J Clin Invest 130, 3894-3908; Soudja, S. M., Ruiz, A. L., Marie, J. C., and Lauvau, G. (2012). Inflammatory monocytes activate memory CD8(+) T and innate NK lymphocytes independent of cognate antigen during microbial pathogen invasion. Immunity 37, 549-562).

While combinations of distinct immune checkpoint inhibitors can provide higher rates of progression-free survival and overall survival, treatment-related adverse events, including skin-related events and severe gastrointestinal symptoms constitute a dangerous threat in patient undergoing ICB combination therapy (Wolchok, J. D., Chiarion-Sileni, V., Gonzalez, R., Rutkowski, P., Grob, J. J., Cowey, C. L., Lao, C. D., Wagstaff, J., Schadendorf, D., Ferrucci, P. F., et al. (2017). Overall Survival with Combined Nivolumab and Ipilimumab in Advanced Melanoma. N Engl J Med 377, 1345-1356).

Alternatively, neo-adjuvant combinations of immune checkpoint inhibitors may represent a promising therapeutic avenue for patients with advanced melanoma and other cancers (Versluis, J. M., Long, G. V., and Blank, C. U. (2020). Learning from clinical trials of neoadjuvant checkpoint blockade. Nat Med 26, 475-484). However, also for such combinations severe side effects can limit completion of the treatment (Amaria, R. N., Reddy, S. M., Tawbi, H. A., Davies, M. A., Ross, M. I., Glitza, I. C., Cormier, J. N., Lewis, C., Hwu, W. J., Hanna, E., et al. (2018). Neoadjuvant immune checkpoint blockade in high-risk resectable melanoma. Nat Med 24, 1649-1654; Blank, C. U., Rozeman, E. A., Fanchi, L. F., Sikorska, K., van de Wiel, B., Kvistborg, P., Krijgsman, O., van den Braber, M., Philips, D., Broeks, A., et al. (2018). Neoadjuvant versus adjuvant ipilimumab plus nivolumab in macroscopic stage Ill melanoma. Nat Med 24, 1655-1661).

In summary, mainstream initiation of immune checkpoint therapy to treat cancers is currently obstructed by the low response rate and immune-related adverse events in some cancer patients. Therefore, factors that could improve the therapeutic outcome are required. For example, single ICB neo-adjuvant therapy in resectable lung cancer was recently reported to be associated with few side effects only and did not delay surgery (Forde, P. M., Chaft, J. E., Smith, K. N., Anagnostou, V., Cottrell, T. R., Hellmann, M. D., Zahurak, M., Yang, S. C., Jones, D. R., Broderick, S., et al. (2018). Neoadjuvant PD-1 Blockade in Resectable Lung Cancer. N Engl J Med 378, 1976-1986). Therefore, substances capable of enhancing neo-adjuvant efficacy of (single) immune checkpoint inhibitors may provide effective therapeutic responses with reduced toxicity.

In view of the above, it is the object of the present invention to overcome the drawbacks of the current immunotherapy with checkpoint inhibitors outlined above and to provide a novel combination of checkpoint inhibitors with agents enhancing the anti-tumor effects of checkpoint inhibitors. Thereby, (i) the fraction of patients responding to treatment with immune checkpoint inhibitors can be increased and/or (ii) the doses of the immune checkpoint inhibitors can be decreased and adverse combinations for enhancing the effects of the checkpoint inhibitors can be avoided to reduce or prevent adverse side effects.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%.

### Combination of a Checkpoint Inhibitor and an ATP-Hydrolyzing Enzyme

In a first aspect the present invention provides a combination of
(i) an immune checkpoint inhibitor; and
(ii) (a) an ATP hydrolyzing enzyme,
   (b) a nucleic acid comprising a polynucleotide encoding the ATP hydrolyzing enzyme,
   (c) a host cell comprising the nucleic acid,
   (d) a microorganism comprising the nucleic acid, or
   (e) a viral particle comprising the nucleic acid.

The present inventors surprisingly found that an ATP-hydrolyzing enzyme (or a host cell/microorganism encoding an ATP-hydrolyzing enzyme) increases the anti-tumor efficacy of immune checkpoint inhibitors, as shown in the appended examples. Accordingly, the combination of an immune checkpoint inhibitor and an ATP-hydrolyzing enzyme—or a nucleic acid encoding an ATP-hydrolyzing enzyme; or a host cell, microorganism or viral particle comprising such a nucleic acid (and, thus, expressing an ATP-hydrolyzing enzyme)—results in more efficient cancer treatment. Thereby, the number of patients responding to anti-cancer treatment with checkpoint inhibitors may be increased. Moreover, the dose of the checkpoint inhibitor may be decreased or adverse combinations of checkpoint inhibitors may be avoided in order to reduce severe side effects.

In the following, the components of the combination according to the present invention, i.e. (i) the immune checkpoint modulator and (ii) the ATP hydrolyzing enzyme, the nucleic acid comprising the polynucleotide encoding the ATP hydrolyzing enzyme, the host cell comprising the nucleic acid, the microorganism comprising the nucleic acid or the viral particle comprising the nucleic acid, are described in detail. It is understood that (i) any embodiment of the immune checkpoint inhibitor as described herein may be combined with (ii) any embodiment of the ATP hydrolyzing enzyme, the nucleic acid comprising the polynucleotide encoding the ATP hydrolyzing enzyme, the host cell comprising the nucleic acid, the microorganism comprising the nucleic acid or the viral particle comprising the nucleic acid as described herein.

### Immune Checkpoint Modulator

As used herein (i.e. throughout the present specification), the term “immune checkpoint modulator” (also referred to as “checkpoint modulator”) refers to a molecule or to a compound that modulates (e.g., totally or partially reduces, inhibits, interferes with, activates, stimulates, increases, reinforces or supports) the function of one or more (immune) checkpoint molecules. In other words, an “immune checkpoint modulator” is a modulator of an immune checkpoint molecule. Thus, an immune checkpoint modulator may be an “immune checkpoint inhibitor” (also referred to as “checkpoint inhibitor” or “inhibitor”) or an “immune checkpoint activator” (also referred to as “checkpoint activator” or “activator”). An “immune checkpoint inhibitor” (also referred to as “checkpoint inhibitor” or “inhibitor”) totally or partially reduces, inhibits, interferes with, or negatively modulates the function of one or more checkpoint molecules. An “immune checkpoint activator” (also referred to as “checkpoint activator” or “activator”) totally or partially activates, stimulates, increases, reinforces, supports or positively modulates the function of one or more checkpoint molecules. Immune checkpoint modulators are typically able to modulate (i) self-tolerance and/or (ii) the amplitude and/or the duration of the immune response. Preferably, the immune checkpoint modulator used according to the present invention modulates the function of one or more human checkpoint molecules and is, thus, a “human checkpoint modulator”. Preferably, the immune checkpoint modulator is an activator or an inhibitor of one or more immune checkpoint point molecule(s) selected from CD27, CD28, CD40, CD122, CD137, OX40, GITR, ICOS, A2AR, B7-H3, B7-H4, BTLA (CD272), CTLA-4, IDO, KIR, LAG3, PD-1, PD-L1, PD-L2, TIM-3, VISTA, CEACAM1, GARP, PS, CSF1R, CD94/NKG2A, TDO, GITR, TNFR, TIGIT and/or FasR/DcR3; or an activator or an inhibitor of one or more ligands thereof.

Checkpoint molecules (also referred to as “immune checkpoint molecules” or “immune checkpoints”) are molecules, such as proteins, which are typically involved in immune pathways and, for example, regulate T-cell activation, T-cell proliferation and/or T-cell function. Immune checkpoint molecules are often referred to as “gate keepers” of the immune system. They are usually crucial for self-tolerance, which prevents the immune system from attacking cells indiscriminately. However, some cancers can protect themselves from immune attacks by stimulating immune checkpoint targets, which in turn prevent or reduce immune responses. In view thereof, it is the goal of an immune checkpoint modulator to modulate an immune checkpoint molecule such that immune responses are not prevented or reduced, but rather elicited or enhanced. Accordingly, the function of checkpoint molecules, which is modulated (e.g., totally or partially reduced, inhibited, interfered with, activated, stimulated, increased, reinforced or supported) by checkpoint modulators, is typically the (regulation of) T-cell activation, T-cell proliferation and/or T cell function. Immune checkpoint molecules thus regulate and maintain self-tolerance and the duration and amplitude of physiological immune responses. Many of the immune checkpoint molecules belong to the B7:CD28 family or to the tumor necrosis factor receptor (TNFR) super family and, by binding to specific ligands, activate signaling molecules that are recruited to the cytoplasmic domain (cf. Susumu Suzuki et al., 2016: Current status of immunotherapy. Japanese Journal of Clinical Oncology, 2016: doi: 10.1093/jjco/hyv201 [Epub ahead of print]; in particular Table 1).

The B7:CD28 family comprises the most frequently targeted pathways in immune checkpoint research including the CTLA-4-B7-1/B7-2 pathway and the PD-1-B7-H1(PDL1)/B7-DC(PD-L2) pathway. Another member of this family is ICOS-ICOSL/B7-H2. Further members of that family include CD28, B7-H3 and B7-H4.

CD28 is constitutively expressed on almost all human CD4+ T cells and on around half of all CD8 T cells. Binding with its two ligands are CD80 (B7-1) and CD86 (B7-2), expressed on dendritic cells, prompts T cell expansion. The co-stimulatory checkpoint molecule CD28 competes with the inhibitory checkpoint molecule CTLA4 for the same ligands, CD80 and CD86 (Buchbinder E. I. and Desai A., 2016: CTLA-4 and PD-1 Pathways—Similarities, Differences and Implications of Their Inhibition; American Journal of Clinical Oncology, 39(1): 98-106).

Cytotoxic T-Lymphocyte-Associated protein 4 (CTLA4; also known as CD152) is a CD28 homolog with much higher binding affinity for B7. The ligands of CTLA-4 are CD80 (B7-1) and CD86 (B7-2), similarly to CD28. However, unlike CD28, binding of CTLA4 to B7 does not produce a stimulatory signal, but prevents the co-stimulatory signal normally provided by CD28. Moreover, CTLA4 binding to B7 is assumed to even produce an inhibitory signal counteracting the stimulatory signals of CD28:B7 and TCR:MHC binding. CTLA-4 is considered as a “leader” of the inhibitory immune checkpoints, as it stops potentially autoreactive T cells at the initial stage of naïve T-cell activation, typically in lymph nodes (Buchbinder E. I. and Desai A., 2016: CTLA-4 and PD-1 Pathways: Similarities, Differences and Implications of Their Inhibition; American Journal of Clinical Oncology, 39(1): 98-106). Preferred checkpoint inhibitors of CTLA4 include the monoclonal antibodies Yervoy® (Ipilimumab; Bristol Myers Squibb) and Tremelimumab (Pfizer/Medlmmune). Further preferred CTLA-4 inhibitors include the anti-CTLA4 antibodies disclosed in WO 2001/014424, in WO 2004/035607, in US 2005/0201994, and in EP 1212422 B1. Further anti-CTLA-4 antibodies that can be used in the context of the present invention include, for example, those described in: U.S. Pat. Nos. 5,811,097, 5,855,887, 6,051,227, 6,984,720, WO 01/14424 WO 00/37504, US 2002/0039581, US 2002/086014, WO 98/42752, U.S. Pat. Nos. 6,682,736 and 6,207,156; as well as in: Hurwitz et al., Proc. Natl. Acad. Sci. USA, 95(17):10067-10071 (1998); Camacho et al., J. Clin. Oncology, 22(145): Abstract No. 2505 (2004) (antibody CP-675206); Mokyr et al., Cancer Res., 58:5301-5304 (1998), in U.S. Pat. Nos. 5,977,318, 6,682,736, 7,109,003, and in U.S. Pat. No. 7,132,281.

Programmed Death 1 receptor (PD1) has two ligands, PD-L1 (also known as B7-H1 and CD274) and PD-L2 (also known as B7-DC and CD273). The PD1 pathway regulates previously activated T cells at the later stages of an immune response, primarily in peripheral tissues. An advantage of targeting PD1 is thus that it can restore immune function in the tumor microenvironment. Preferred inhibitors of the PD1 pathway include Opdivo® (Nivolumab; Bristol Myers Squibb), Keytruda® (Pembrolizumab; Merck), Durvalumab (Medlmmune/AstraZeneca), MED14736 (AstraZeneca; as described in WO 2011/066389 A1), Atezolizumab (MPDL3280A, Roche/Genentech; cf. U.S. Pat. No. 8,217,149 B2), Pidilizumab (CT-011; CureTech), MED10680 (AMP-514; AstraZeneca), Avelumab (Merck), MSB-0010718C (Merck), PDR001 (Novartis), BMS-936559 (Bristol Myers Squibb), REGN2810 (Regeneron Pharmaceuticals), MIH1 (Affymetrix), AMP-224 (Amplimmune, GSK), BGB-A317 (BeiGene) and Lambrolizumab (e.g. disclosed as hPD109A and its humanized derivatives h409A11, h409A16 and h409A17 in WO2008/156712; Hamid et al., 2013; N. Engl. J. Med. 369: 134-144).

Inducible T-cell costimulator (ICOS; also known as CD278) is expressed on activated T cells. Its ligand is ICOSL (B7-H2; CD275), expressed mainly on B cells and dendritic cells. The molecule seems to be important in T cell effector function.

B7-H3 (also known as CD276) was originally understood to be a co-stimulatory molecule but is now regarded as co-inhibitory. A preferred checkpoint inhibitor of B7-H3 is the Fc-optimized monoclonal antibody Enoblituzumab (MGA271; MacroGenics; cf. US 2012/0294796 A1).

B7-H4 (also known as VTCN1), is expressed by tumor cells and tumor-associated macrophages and plays a role in tumor escape. Preferred B7-H4 inhibitors are the antibodies described in Dangaj, D. et al., 2013; Cancer Research 73(15): 4820-9 and in Table 1 and the respective description of Jenessa B. Smith et al., 2014: B7-H4 as a potential target for immunotherapy for gynecologic cancers: A closer look. Gynecol Oncol 134(1): 181-189. Other preferred examples of B7-H4 inhibitors include antibodies to human B7-H4 as disclosed, e.g., in WO 2013/025779 A1 and in WO 2013/067492 A1 or soluble recombinant forms of B7-H4, such as disclosed in US 2012/0177645 A1.

The TNF superfamily comprises in particular 19 protein-ligands binding to 29 cytokine receptors. They are involved in many physiological responses such as apoptosis, inflammation or cell survival (Croft, M., C. A. Benedict, and C. F. Ware, Clinical targeting of the TNF and TNFR superfamilies. Nat Rev Drug Discov, 2013.12(2): p. 147-68). The following checkpoint molecules/pathways are preferred for cancer indications: TNFRSF4 (OX40/OX40L), TNFRSFS (CD40L/CD40), TNFRSF7 (CD27/CD70), TNFRSF8 (CD30/CD30L), TNFRSF9 (4-1BB/4-1BBL), TNFRSF10 (TRAILR/TRAIL)), TNFRSF12 (FN14/TWEAK), TNFRSF13 (BAFFRTACI/APRIL-BAFF) and TNFRSF18 (GITR/GITRL). Further preferred checkpoint molecules/pathways include Fas-Ligand and TNFRSF1 (TNFα/TNFR). Moreover, the B- and T-lymphocyte attenuator (BTLA)/herpes virus entry mediator (HVEM) pathway are preferred for enhancing immune responses, just like the CTLA-4 blockade. Accordingly, in the context of the present invention such checkpoint modulators are preferred for the use in the treatment and/or prevention in cancer, which modulate one or more checkpoint molecules selected from TNFRSF4 (OX40/0X40L), TNFRSFS (CD40L/CD40), TNFRSF7 (CD27/CD70), TNFRSF9 (4-1BB/4-1BBL), TNFRSF18 (GITR/GITRL), FasR/DcR3/Fas ligand, TNFRSF1 (TNFα/TNFR), BTLA/HVEM and CTLA4.

OX40 (also known as CD134 or TNFRSF4) promotes the expansion of effector and memory T cells, but it is also able to suppress the differentiation and activity of T-regulatory cells and to regulate cytokine production. The ligand of OX40 is OX40L (also known as TNFSF4 or CD252). OX40 is transiently expressed after T-cell receptor engagement and is only upregulated on the most recently antigen-activated T cells within inflammatory lesions. Preferred checkpoint modulators of OX40 include MED16469 (Medlmmune/AstraZeneca), MED16383 (Medlmmune/AstraZeneca), MED10562 (Medlmmune/AstraZeneca), MOXR0916 (RG7888; Roche/Genentech) and GSK3174998 (GSK).

CD40 (also known as TNFRSF5) is expressed by a variety of immune system cells including antigen presenting cells. Its ligand is CD40L, also known as CD154 or TNFSF5, is transiently expressed on the surface of activated CD4+ T cells. CD40 signaling “licenses” dendritic cells to mature and thereby trigger T-cell activation and differentiation. However, CD40 can also be expressed by tumor cells. Thus, stimulation/activation of CD40 in cancer patients can be beneficial or deleterious. Accordingly, stimulatory and inhibitory modulators of this immune checkpoint were developed (Sufia Butt Hassan, Jesper Freddie Sorensen, Barbara Nicola Olsen and Anders Elm Pedersen, 2014: Anti-CD40-mediated cancer immunotherapy: an update of recent and ongoing clinical trials, Immunopharmacology and Immunotoxicology, 36:2, 96-104). Preferred examples of CD40 checkpoint modulators include (i) agonistic anti-CD antibodies as described in Sufia Butt Hassan, Jesper Freddie Sorensen, Barbara Nicola Olsen and Anders Elm Pedersen, 2014: Anti-CD40-mediated cancer immunotherapy: an update of recent and ongoing clinical trials, Immunopharmacology and Immunotoxicology, 36:2, 96-104, such as Dacetuzumab (SGN-40), CP-870893, FGK 4.5/FGK 45 and FGK115, preferably Dacetuzumab, and (ii) antagonistic anti-CD antibodies as described in Sufia Butt Hassan, Jesper Freddie Sorensen, Barbara Nicola Olsen and Anders Elm Pedersen, 2014: Anti-CD40-mediated cancer immunotherapy: an update of recent and ongoing clinical trials, Immunopharmacology and Immunotoxicology, 36:2, 96-104, such as Lucatumumab (HCD122, CHIR-12.12). Further preferred immune checkpoint modulators of CD40 include SEA-CD40 (Seattle Genetics), ADC-1013 (Alligator Biosciences), APX005M (Apexigen Inc) and RO7009789 (Roche).

CD27 (also known as TNFRSF7) supports antigen-specific expansion of naïve T cells and plays an important role in the generation of T cell memory. CD27 is also a memory marker of B cells. The transient availability of its ligand, CD70 (also known as TNFSF7 or CD27L), on lymphocytes and dendritic cells regulates the activity of CD27. Moreover, CD27 co-stimulation is known to suppress Th17 effector cell function. A preferred immune checkpoint modulator of CD27 is Varlilumab (Celldex). Preferred immune checkpoint modulators of CD70 include ARGX-110 (arGEN-X) and SGN-CD70A (Seattle Genetics).

CD137 (also known as 4-1 BB or TNFRSF9) is a member of the tumor necrosis factor (TNF) receptor family and is increasingly associated with costimulatory activity for activated T cells. In particular, CD137 signaling (via its ligand CD137L, also known as TNFSF9 or 4-1BBL) results in T-cell proliferation and protects T cells, in particular, CD8⁺ T cells, from activation-induced cell death. Preferred checkpoint modulators of CD137 include PF-05082566 (Pfizer) and Urelumab (BMS).

Glucocorticoid-Induced TNFR family Related gene (GITR, also known as TNFRSF18), prompts T cell expansion, including Treg expansion. The ligand for GITR (GITRL, TNFSF18) is mainly expressed on antigen presenting cells. Antibodies to GITR have been shown to promote an anti-tumor response through loss of Treg lineage stability. Preferred checkpoint modulators of GITR include BMS-986156 (Bristol Myers Squibb), TRX518 (GITR Inc) and MK-4166 (Merck).

B and T Lymphocyte Attenuator (BTLA; also known as CD272) is in particular expressed by CD8⁺ T cells, wherein surface expression of BTLA is gradually downregulated during differentiation of human CD8⁺ T cells from the naive to effector cell phenotype. However, tumor-specific human CD8⁺ T cells express high levels of BTLA. BTLA expression is induced during activation of T cells, and BTLA remains expressed on Th1 cells but not Th2 cells. Like PD1 and CTLA4, BTLA interacts with a B7 homolog, B7H4. However, unlike PD-1 and CTLA-4, BTLA displays T-Cell inhibition via interaction with tumor necrosis family receptors (TNF-R), not just the B7 family of cell surface receptors. BTLA is a ligand for tumor necrosis factor (receptor) superfamily, member 14 (TNFRSF14), also known as herpes virus entry mediator (HVEM; Herpesvirus Entry Mediator, also known as CD270). BTLA-HVEM complexes negatively regulate T-cell immune responses. Preferred BTLA inhibitors are the antibodies described in Table 1 of Alison Crawford and E. John Wherry, 2009: Editorial: Therapeutic potential of targeting BTLA. Journal of Leukocyte Biology 86: 5-8, in particular the human antibodies thereof. Other preferred antibodies in this context, which block human BTLA interaction with its ligand are disclosed in WO 2011/014438, such as “4C7” as described in WO 2011/014438.

Another checkpoint molecule family includes checkpoint molecules related to the two primary class of major histocompatibility complex (MHC) molecules (MHC class I and class 1l). This family includes killer Ig-like Receptor (KIR) for class I and lymphocyte activation gene-3 (LAG-3) for class 1l.

Killer-cell Immunoglobulin-like Receptor (KIR) is a receptor for MHC Class I molecules on Natural Killer cells. An exemplary inhibitor of KIR is the monoclonal antibody Lirilumab (IPH 2102; Innate Pharma/BMS; cf. U.S. Pat. No. 8,119,775 B2 and Benson et al., 2012, Blood 120:4324-4333).

Lymphocyte Activation Gene-3 (LAG3, also known as CD223) signaling leads to suppression of an immune response by action to Tregs as well as direct effects on CD8⁺ T cells. A preferred example of a LAG3 inhibitor is the anti-LAG3 monoclonal antibody BMS-986016 (Bristol-Myers Squibb). Other preferred examples of a LAG3 inhibitor include LAG525 (Novartis), IMP321 (Immutep) and LAG3-Ig as disclosed in WO 2009/044273 A2 and in Brignon et al., 2009, Clin. Cancer Res. 15: 6225-6231 as well as mouse or humanized antibodies blocking human LAG3 (e.g., IMP701 as described in WO 2008/132601 A1), or fully human antibodies blocking human LAG3 (such as disclosed in EP 2320940 A2).

Another checkpoint molecule pathway is the TIM-3/GAL9 pathway. T-cell Immunoglobulin domain and Mucin domain 3 (TIM-3, also known as HAVcr-2) is expressed on activated human CD4+ T cells and regulates Th1 and Th17 cytokines. TIM-3 acts as a negative regulator of Th1/Tc1 function by triggering cell death upon interaction with its ligand, galectin-9 (GAL9). TIM-3 is a T helper type 1 specific cell surface molecule that is regulating the induction of peripheral tolerance. A recent study has indeed demonstrated that TIM-3 antibodies could significantly enhance antitumor immunity (Ngiow, S. F., et al., Anti-TIM3 antibody promotes T cell IFN-gammamediated antitumor immunity and suppresses established tumors. Cancer Res, 2011. 71(10): p. 3540-51). Preferred examples of TIM-3 inhibitors include antibodies targeting human TIM3 (e.g. as disclosed in WO 2013/006490 A2) or, in particular, the anti-human TIM3 blocking antibody F38-2E2 as disclosed by Jones et al., 2008, J Exp Med. 205 (12): 2763-79.

CEACAM1 (Carcinoembryonic antigen-related cell adhesion molecule 1) is a further checkpoint molecule (Huang, Y. H., et al., CEACAM1 regulates TIM-3-mediated tolerance and exhaustion. Nature, 2015. 517(7534): p. 386-90; Gray-Owen, S. D. and R. S. Blumberg, CEACAM1: contact-dependent control of immunity. Nat Rev Immunol, 2006. 6(6): p. 433-46). A preferred checkpoint modulator of CEACAM1 is CM-24 (cCAM Biotherapeutics).

Another immune checkpoint molecule is GARP, which plays a role in the ability of tumors to escape the patient's immune system. Presently in clinical trials, the candidate (ARGX-115) seems demonstrating interesting effect. Accordingly, ARGX-115 is a preferred GARP checkpoint modulator.

Moreover, various research groups have demonstrated that another checkpoint molecule is phosphatidylserine (also referred to as “PS”) may be targeted for cancer treatment (Creelan, B. C., Update on immune checkpoint inhibitors in lung cancer. Cancer Control, 2014. 21(1): p. 80-9; Yin, Y., et al., Phosphatidylserine-targeting antibody induces Ml macrophage polarization and promotes myeloid-derived suppressor cell differentiation. Cancer Immunol Res, 2013. 1(4): p. 256-68). A preferred checkpoint modulator of phosphatidylserine (PS) is Bavituximab (Peregrine).

Another checkpoint pathway is CSF1/CSF1R (Zhu, Y., et al., CSF1/CSF1R Blockade Reprograms Tumor-Infiltrating Macrophages and Improves Response to T-cell Checkpoint Immunotherapy in Pancreatic Cancer Models. Cancer Research, 2014. 74(18): p. 5057-5069). Preferred checkpoint modulators of CSF1R include FPA008 (FivePrime), IMC-CS4 (Eli-Lilly), PLX3397 (Plexxicon) and RO5509554 (Roche).

Furthermore, the CD94/NKG2A natural killer cell receptor is evaluated for its role in cervical carcinoma (Sheu, B. C., et al., Up-regulation of inhibitory natural killer receptors CD94/NKG2A with suppressed intracellular perforin expression of tumor infiltrating CD8+ T lymphocytes in human cervical carcinoma. Cancer Res, 2005. 65(7): p. 2921-9) and in leukemia (Tanaka, J., et al., Cytolytic activity against primary leukemic cells by inhibitory NK cell receptor (CD94/NKG2A)-expressing T cells expanded from various sources of blood mononuclear cells. Leukemia, 2005. 19(3): p. 486-9). A preferred checkpoint modulator of NKG2A is IPH2201 (Innate Pharma).

Another checkpoint molecule is IDO, the indoleamine 2,3-dioxygenase enzyme of the kynurenine pathway (Ball, H. J., et al., Indoleamine 2,3-dioxygenase-2; a new enzyme in the kynurenine pathway. Int J Biochem Cell Biol, 2009. 41(3): p. 467-71). Indoleamine 2,3-dioxygenase (IDO) is a tryptophan catabolic enzyme with immune-inhibitory properties. IDO is known to suppress T and NK cells, generate and activate Tregs and myeloid-derived suppressor cells, and promote tumour angiogenesis. IDO1 is overexpressed in many cancer and was shown to allow tumor cells escaping from the immune system (Liu, X., et al., Selective inhibition of ID01 effectively regulates mediators of antitumor immunity. Blood, 2010. 115(17): p. 3520-30; Ino, K., et al., Inverse correlation between tumoral indoleamine 2,3-dioxygenase expression and tumor-infiltrating lymphocytes in endometrial cancer: its association with disease progression and survival. Clin Cancer Res, 2008. 14(8): p. 2310-7) and to facilitate chronic tumor progression when induced by local inflammation (Muller, A. J., et al., Chronic inflammation that facilitates tumor progression creates local immune suppression by inducing indoleamine 2,3 dioxygenase. Proc Natl Acad Sci US A, 2008. 105(44): p. 17073-8). Preferred IDO inhibitors include Exiguamine A, epacadostat (INCB024360; InCyte), Indoximod (NewLink Genetics), NLG919 (NewLink Genetics/Genentech), GDC-0919 (NewLink Genetics/Genentech), F001287 (Flexus Biosciences/BMS) and small molecules such as 1-methyl-tryptophan, in particular 1-methyl-[D]-tryptophan and the IDO inhibitors listed in Table 1 of Sheridan C., 2015: IDO inhibitors move center stage in immune-oncology; Nature Biotechnology 33: 321-322.

Another immune checkpoint molecule, which may be modulated is also a member of the kynurenine metabolic pathway: TDO (tryptophan-2,3-dioxygenase). Several studies already demonstrated the interest of TDO in cancer immunity and autoimmunity (Garber, K., Evading immunity: new enzyme implicated in cancer. J Natl Cancer Inst, 2012. 104(5): p. 349-52; Platten, M., W. Wick, and B. J. Van den Eynde, Tryptophan catabolism in cancer: beyond !DO and tryptophan depletion. Cancer Res, 2012. 72(21): p. 5435-40; Platten, M., et al., Cancer Immunotherapy by Targeting IDO/TDO and Their Downstream Effectors. Front Immunol, 2014. 5: p. 673).

Another immune checkpoint molecule, which may be modulated is A2AR. The Adenosine A2A receptor (A2AR) is regarded as an important checkpoint in cancer therapy because the tumor microenvironment has typically relatively high concentrations of adenosine, which is activating A2AR. Such signaling provides a negative immune feedback loop in the immune microenvironment (for review see Robert D. Leone et al., 2015: A2aR antagonists: Next generation checkpoint blockade for cancer immunotherapy. Computational and Structural Biotechnology Journal 13: 265-272). Preferred A2AR inhibitors include Istradefylline, PBS-509, ST1535, ST4206, Tozadenant, V81444, Preladenant, Vipadenant, SCH58261, SYN115, ZM241365 and FSPTP.

Another immune checkpoint molecule, which may be modulated is VISTA. V-domain Ig suppressor of T cell activation (VISTA; also known as C10orf54) is primarily expressed on hematopoietic cells so that consistent expression of VISTA on leukocytes within tumors may allow VISTA blockade to be effective across a broad range of solid tumors. A preferred VISTA inhibitor is JNJ-61610588 (ImmuNext), an anti-VISTA antibody, which recently entered a phase 1 clinical trial.

Another immune checkpoint molecule is CD122. CD122 is the Interleukin-2 receptor beta sub-unit. CD122 increases proliferation of CD8+ effector T cells.

Recently, T cell immunoglobulin and ITIM domain (TIGIT) emerged as immune checkpoint molecule. TIGIT is an inhibitory receptor expressed on lymphocytes, which interacts with CD155 expressed on antigen-presenting cells or tumor cells to downregulate T cell and natural killer (NK) cell functions. TIGIT action and effects of TIGIT blockade are described, for example, in Harjunpää H, Guillerey C. TIGIT as an emerging immune checkpoint. Clin Exp Immunol. 2020; 200(2):108-119. doi:10.1111/cei.13407, which is incorporated herein in its entirety. TIGIT blockade may be combined with blockade of the PD1-pathway or may be used as sole checkpoint inhibitor treatment. Exemplified antibodies for blockade of TIGIT, which may be used as sole checkpoint inhibitor or in combination with an inhibitor (antibody) of the PD1 pathway, include, but are not limited to, Etigilimab (OMP-313M32), Tiragolumab (MTIG7192A; RG6058), AB154 (Arcus Bioscience), MK-7684, BMS-986207, ASP8374, and ASP8374.

Immune checkpoint molecules are responsible for co-stimulatory or inhibitory interactions of T-cell responses. Accordingly, checkpoint molecules can be divided into (i) (co-)stimulatory checkpoint molecules and (ii) inhibitory checkpoint molecules. Typically, (co-)stimulatory checkpoint molecules act positively in concert with T-cell receptor (TCR) signaling induced by antigen stimulation, whereas inhibitory checkpoint molecules negatively regulate TCR signaling. Examples of (co-)stimulatory checkpoint molecules include CD27, CD28, CD40, CD122, CD137, OX40, GITR and ICOS. Examples of inhibitory checkpoint molecules include CTLA4 as well as PD1 with its ligands PD-L1 and PD-L2; and A2AR, B7-H3, B7-H4, BTLA, IDO, KIR, LAG3, TIM-3, VISTA, CEACAM1, GARP, PS, CSF1R, CD94/NKG2A, TDO, TNFR, TIGIT and FasR/DcR3.

Preferably, the immune checkpoint modulator is an activator of a (co-)stimulatory checkpoint molecule or an inhibitor of an inhibitory checkpoint molecule or a combination thereof. For example, the immune checkpoint modulator may be (i) an activator of CD27, CD28, CD40, CD122, CD137, OX40, GITR and/or ICOS or (ii) an inhibitor of A2AR, B7-H3, B7-H4, BTLA, CD40, CTLA-4, IDO, KIR, LAG3, PD-1, PDL-1, PD-L2, TIM-3, VISTA, CEACAM1, GARP, PS, CSF1R, CD94/NKG2A, TDO, TNFR, TIGIT and/or FasR/DcR3.

As described above, a number of modulators of CD27, CD28, CD40, CD122, CD137, OX40, GITR, ICOS, A2AR, B7-H3, B7-H4, CTLA-4, PD1, PDL-1, PD-L2, IDO, LAG-3, BTLA, TIM3, VISTA, KIR, CEACAM1, GARP, PS, CSF1R, CD94/NKG2A, TDO, TNFR, TIGIT and/or FasR/DcR3 are known to the skilled person. Some are in clinical trials or even approved by some authorities (in some countries). Based on these known immune checkpoint modulators, alternative immune checkpoint modulators may be developed in the (near) future. In particular, known modulators of the preferred immune checkpoint molecules may be used as such, or analogues thereof may be used, in particular chimerized, humanized or human forms of antibodies.

Preferably, the immune checkpoint modulator is an inhibitor of an inhibitory checkpoint molecule (but no inhibitor of a stimulatory checkpoint molecule). The inhibitory checkpoint molecule may be selected from A2AR, B7-H3, B7-H4, BTLA, CD40, CTLA-4, IDO, KIR, LAG3, PD-1, PDL-1, PD-L2, TIM-3, VISTA, CEACAM1, GARP, PS, CSF1R, CD94/NKG2A, TDO, TNFR, TIGIT and FasR/DcR3. In certain embodiments, the immune checkpoint modulator may be an inhibitor of A2AR, B7-H3, B7-H4, BTLA, CTLA-4, IDO, KIR, LAG3, PD-1, TIM-3, VISTA, CEACAM1, GARP, PS, CSF1R, CD94/NKG2A, TDO, TNFR, TIGIT and/or DcR3 or of a ligand thereof.

In some embodiments, the immune checkpoint modulator may be an activator of a stimulatory or costimulatory checkpoint molecule (but preferably no activator of an inhibitory checkpoint molecule). For example, the immune checkpoint modulator may be an activator of CD27, CD28, CD40, CD122, CD137, OX40, GITR and/or ICOS or of a ligand thereof.

More preferably, the immune checkpoint modulator is an inhibitor of the “CTLA4-pathway” or an inhibitor of the “PD1-pathway”, including CTLA4 and its ligands CD80 and CD86 and PD1 with its ligands PD-L1 and PD-L2, respectively (more details on CTLA4 and PD-1 pathways as well as further participants are described in Buchbinder E. I. and Desai A., 2016: CTLA-4 and PD-1 Pathways—Similarities, Differences and Implications of Their Inhibition; American Journal of Clinical Oncology, 39(1): 98-106). In some embodiments, the immune checkpoint modulator is an inhibitor of CTLA-4, PD-1, PD-L1 and/or PD-L2, preferably an inhibitor of PD-1, PD-L1 and/or PD-L2, more preferably the immune checkpoint modulator is an inhibitor of PD-L1 and/or PD-1, and even more preferably an inhibitor of PD-L1.

Accordingly, the checkpoint modulator may be selected from known inhibitors of the CTLA-4 pathway and/or the PD-1 pathway. Preferred inhibitors of the CTLA-4 pathway and of the PD-1 pathway include the monoclonal antibodies Yervoy® (Ipilimumab; Bristol Myers Squibb) and Tremelimumab (Pfizer/Medlmmune) as well as Opdivo® (Nivolumab; Bristol Myers Squibb), Keytruda® (Pembrolizumab; Merck), Durvalumab (Medlmmune/AstraZeneca), MED14736 (AstraZeneca; cf. WO 2011/066389 A1), MPDL3280A (Roche/Genentech; cf. U.S. Pat. No. 8,217,149 B2), Pidilizumab (CT-011; CureTech), MEDI0680 (AMP-514; AstraZeneca), MSB-0010718C (Merck), MIH1 (Affymetrix) and Lambrolizumab (e.g. disclosed as hPD109A and its humanized derivatives h409A11, h409A16 and h409A17 in WO2008/156712; Hamid et al., 2013; N. Engl. J. Med. 369: 134-144). More preferred checkpoint inhibitors include the CTLA-4 inhibitors Yervoy® (Ipilimumab; Bristol Myers Squibb) and Tremelimumab (Pfizer/Medlmmune) and/or the PD-1 inhibitors Opdivo® (Nivolumab; Bristol Myers Squibb), Keytruda® (Pembrolizumab; Merck), Pidilizumab (CT-011; CureTech), MED10680 (AMP-514; AstraZeneca), AMP-224 and Lambrolizumab (e.g. disclosed as hPD109A and its humanized derivatives h409A11, h409A16 and h409A17 in WO2008/156712; Hamid O. et al., 2013; N. Engl. J. Med. 369: 134-144).

In some embodiments, the combination of the invention comprises a single immune checkpoint modulator only. Alternatively, more than one immune checkpoint modulator (e.g., checkpoint inhibitor) may be used, in particular at least 2, 3, 4, 5, 6, 7, 8, 9 or 10 distinct immune checkpoint modulators (e.g., checkpoint inhibitors) may be used, e.g. (exactly) 2 distinct immune checkpoint modulators (e.g., checkpoint inhibitors) are used. In some embodiments, the distinct immune checkpoint modulators (e.g., checkpoint inhibitors) used in combination modulate (e.g., inhibit) different checkpoint pathways. For example, an inhibitor of the PD-1 pathway may be combined with an inhibitor of the CTLA-4 pathway. In other embodiments, the distinct immune checkpoint modulators (e.g., checkpoint inhibitors) used in combination modulate (e.g., inhibit) the same checkpoint pathway.

In the context of the present invention immune checkpoint modulators may be any kind of molecule or agent, as long as it totally or partially reduces, inhibits, interferes with, activates, stimulates, increases, reinforces or supports the function of one or more checkpoint molecules as described above. In particular, the immune checkpoint modulator binds to one or more checkpoint molecules, such as checkpoint proteins, or to its precursors, e.g. on DNA- or RNA-level, thereby modulating (e.g., totally or partially reducing, inhibiting, interfering with, activating, stimulating, increasing, reinforcing or supporting) the function of one or more checkpoint molecules as described above. For example, immune checkpoint modulators may be oligonucleotides, siRNA, shRNA, ribozymes, anti-sense RNA molecules, immunotoxins, small molecule inhibitors and antibodies or antigen binding fragments thereof (e.g., checkpoint molecule blocking antibodies or antibody fragments, antagonist antibodies or antibody fragments or agonist antibodies or antibody fragments).

In certain embodiments, the immune checkpoint modulator may be an oligonucleotide. Such an oligonucleotide may be used to decrease protein expression, in particular to decrease the expression of a checkpoint protein, such as the checkpoint receptors or ligands described above. Oligonucleotides are short DNA or RNA molecules, typically comprising from 2 to 50 nucleotides, preferably from 3 to 40 nucleotides, more preferably from 4 to 30 nucleotides and even more preferably from 5 to 25 nucleotides, such as, for example 4, 5, 6, 7, 8, 9 or 10 nucleotides. Oligonucleotides are usually made in the laboratory by solid-phase chemical synthesis. Oligonucleotides maybe single-stranded or double-stranded, however, in the context of the present invention the oligonucleotide may be single-stranded. In some embodiments, the checkpoint modulator oligonucleotide is an antisense-oligonucleotide. Antisense-oligonucleotides are single strands of DNA or RNA that are complementary to a chosen sequence, in particular to a sequence chosen from the DNA or RNA sequence (or a fragment thereof) of a checkpoint protein. Antisense RNA is typically used to prevent protein translation of messenger RNA strands, e.g. of mRNA for a checkpoint protein, by binding to the mRNA. Antisense DNA is typically used to target a specific, complementary (coding or non-coding) RNA. If binding takes place, such a DNA/RNA hybrid can be degraded by the enzyme RNase H. Moreover, morpholino-antisense oligonucleotides can be used for gene knockdowns in vertebrates. For example, Kryczek et al., 2006 (Kryczek I, Zou L, Rodriguez P, Zhu G, Wei S, Mottram P, et al. B7-H4 expression identifies a novel suppressive macrophage population in human ovarian carcinoma. J Exp Med. 2006; 203:871-81) designed a B7-H4-specific morpholino that specifically blocked B7-H4 expression in macrophages, resulting in increased T-cell proliferation and reduced tumor volumes in mice with tumor associated antigen (TAA)-specific T cells.

In some embodiments, the immune checkpoint modulator may be an siRNA. Small interfering RNA (siRNA), sometimes known as short interfering RNA or silencing RNA, is a class of double-stranded RNA molecules, which is typically 20-25 base pairs in length. In the RNA interference (RNAi) pathway, siRNA interferes with the expression of specific genes, such as genes coding for checkpoint proteins, with complementary nucleotide sequences. siRNA functions by causing mRNA to be broken down after transcription, resulting in no translation. Transfection of exogenous siRNA may be used for gene knockdown, however, the effect maybe only transient, especially in rapidly dividing cells. This may be overcome, for example, by RNA modification or by using an expression vector for the siRNA. The siRNA sequence may also be modified to introduce a short loop between the two strands. The resulting transcript is a short hairpin RNA (shRNA, also “small hairpin RNA”), which can be processed into a functional siRNA by Dicer in its usual fashion. shRNA is an advantageous mediator of RNAi in that it has a relatively low rate of degradation and turnover. Accordingly, the immune checkpoint modulator may be an shRNA. shRNA typically requires the use of an expression vector, e.g. a plasmid or a viral or bacterial vector.

In some embodiments, the immune checkpoint modulator may be an immunotoxin. Immunotoxins are chimeric proteins that contain a targeting moiety (such as an antibody), which is typically targeting an antigen on a certain cell, such as a cancer cell, linked to a toxin. In some embodiments, the immunotoxin may comprise a targeting moiety, which targets a checkpoint molecule. When the immunotoxin binds to a cell carrying the antigen, e.g. the checkpoint molecule, it is taken in through endocytosis, and the toxin can then kill the cell. Immunotoxins may comprise a (modified) antibody or antibody fragment, linked to a (fragment of a) toxin. For linkage, methods are well known in the art. The targeting portion of the immunotoxin typically comprises a Fab portion of an antibody that targets a specific cell type. The toxin is usually cytotoxic, such as a protein derived from a bacterial or plant protein, from which the natural binding domain has been removed so that the targeting moiety of the immunotoxin directs the toxin to the antigen on the target cell. However, immunotoxins can also comprise a targeting moiety other than an antibody or antibody fragment, such as a growth factor. For example, recombinant fusion proteins containing a toxin and a growth factor are also referred to as recombinant immunotoxins. In certain embodiments, the immune checkpoint modulator may be a small molecule drug (also referred to as “small molecule inhibitor”). A small molecule drug is a low molecular weight (up to 900 daltons) organic compound that typically interacts with (the regulation of) a biological process. In the context of the present invention, a small molecule drug which is an immune checkpoint modulator, is an organic compound having a molecular weight of no more than 900 daltons, which totally or partially reduces, inhibits, interferes with, or negatively modulates the function of one or more checkpoint molecules as described above. The upper molecular weight limit of 900 daltons allows for the possibility to rapidly diffuse across cell membranes and for oral bioavailability. In some instances, the molecular weight of the small molecule drug which is an immune checkpoint modulator, is no more than 500 daltons. For example, various A2AR antagonists known in the art are organic compounds having a molecular weight below 500 daltons.

Preferably, the immune checkpoint modulator is an antibody or an antigen-binding fragment thereof. Such immune checkpoint modulator antibodies or antigen-binding fragments thereof include in particular antibodies, or antigen binding fragments thereof, that bind to immune checkpoint receptors or antibodies that bind to immune checkpoint receptor ligands. Immune checkpoint modulator antibodies or an antigen-binding fragments thereof may be agonists or antagonists of immune checkpoint receptors or of immune checkpoint receptor ligands. Examples of antibody-type checkpoint modulators include immune checkpoint modulators, which are currently approved, namely, Yervoy® (Ipilimumab; Bristol Myers Squibb), Opdivo® (Nivolumab; Bristol Myers Squibb) and Keytruda® (Pembrolizumab; Merck) and further anti-checkpoint receptor antibodies or anti-checkpoint ligand antibodies as described above.

Preferably, the immune checkpoint modulator in the combination according to the present invention is an antibody or an antigen-binding fragment that can partially or totally block the PD-1 pathway (e.g., they can be partial or full antagonists of the PD-1 pathway), in particular PD-1, PD-L1 or PD-L2. This pathway and examples of antibodies blocking this pathway are described in Ohaegbulam K C, Assal A, Lazar-Molnar E, Yao Y, Zang X. Human cancer immunotherapy with antibodies to the PD-1 and PD-L1 pathway. Trends Mol Med. 2015; 21(1):24-33. doi:10.1016/j.molmed.2014.10.009. In general, antibodies or antigen-binding fragments blocking the PD-1 pathway include anti-PD-1 antibodies, human anti-PD-1 antibodies, mouse anti-PD-1 antibodies, mammalian anti-PD-1 antibodies, humanized anti-PD-1 antibodies, monoclonal anti-PD-1 antibodies, polyclonal anti-PD-1 antibodies, chimeric anti-PD-1 antibodies, anti-PD-L1 antibodies, anti-PD-L2 antibodies, anti-PD-1 adnectins, anti-PD-1 domain antibodies, single chain anti-PD-1 fragments, heavy chain anti-PD-1 fragments, and light chain anti-PD-1 fragments. For example, the anti-PD-1 antibody may be an antigen-binding fragment. Preferably, the immune checkpoint modulator antibody is able to bind to human PD-L1 and to partially or totally block the activity of (human) PD-L1 (e.g., they can be partial or full antagonists of PD-L1), thereby in particular unleashing the function of immune cells expressing PD-1 or PD-L1. Examples of antibodies targeting PD-1 include CT-011 (Pidilizumab; CureTech), MK-3475 (Lambrolizumab, Pembrolizumab; Merck), BMS-936558 (Nivolumab; Bristol-Meyers Squibb), and AMP-224 (Amplimmune/GlaxoSmithKline). Examples of antibodies targeting PD-L1 include BMS-936559 (Bristol-Meyers Squibb), MED14736 (Medlmmune), MPDL3280A (Roche) and MSB0010718C (Merck).

In some embodiments, the immune checkpoint modulators in the combination according to the present invention may be antibodies or antigen-binding fragments that can partially or totally block the CTLA-4 pathway (e.g., they can be partial or full antagonists of the CTLA-4 pathway). Such antibodies or antigen-binding fragments include anti-CTLA4 antibodies, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, chimeric anti-CTLA4 antibodies, MDX-010 (ipilimumab), tremelimumab, anti-CD28 antibodies, anti-CTLA4 adnectins, anti-CTLA4 domain antibodies, single chain anti-CTLA4 fragments, heavy chain anti-CTLA4 fragments, and light chain anti-CTLA4 fragments. For example, the anti-CTLA4 antibody may be an antigen-binding fragment. Preferably, the anti-CTLA4 antibody is able to bind to human CTLA4 and to partially or totally block the activity of CTLA4 (e.g., they can be partial or full antagonists of CTLA-4), thereby in particular unleashing the function of immune cells expressing CTLA4.

### ATP-Hydrolyzing Enzymes and Nucleic Acids Encoding ATP-Hydrolyzing Enzymes

According to a first aspect of the present invention, (i) the immune checkpoint modulator is combined with (ii) an ATP-hydrolyzing enzyme.

As used herein, the term “ATP-hydrolyzing enzyme” refers to any enzyme which catalyzes the hydrolysis of ATP to ADP, ATP to AMP and/or ADP to AMP. Such enzymes include but are not limited to apyrase, ATPase, ATP-diphosphatase, adenosine diphosphatase, ADPase, ATP-diphosphohydrolase and CD39 (Ectonucleoside triphosphate diphosphohydrolase 1, ENTPD1). In the context of the present invention, any ATP-hydrolyzing enzyme may be used.

In some embodiments, the ATP-hydrolyzing enzyme is not endogenous CD39 (Ectonucleoside triphosphate diphosphohydrolase 1, ENTPD1). Endogenous CD39 is an integral membrane protein that hydrolyses ATP and ADP in a calcium and magnesium dependent reaction generating AMP. It is activated upon glycosylation and translocation to the cell surface membrane where it displays its enzyme activity as an ectonucleotidase. CD39 is attached to the plasma membrane by two transmembrane domains (Grinthal A, Guidotti G. CD39, NTPDase 1, is attached to the plasma membrane by two transmembrane domains. Why?. Purinergic Signal. 2006; 2(2):391-398. doi:10.1007/s11302-005-5907-8). However, as described below, in the context of the present invention soluble (not membrane-bound) ATP-hydrolyzing enzymes are preferred. In contrast to membrane-bound endogenous CD39, CD39 can be engineered to obtain a soluble form of CD39 (Gayle R B 3rd, Maliszewski C R, Gimpel S D, Schoenborn M A, Caspary R G, Richards C, Brasel K, Price V, Drosopoulos J H, Islam N, Alyonycheva T N, Broekman M J, Marcus A J. Inhibition of platelet function by recombinant soluble ecto-ADPase/CD39. J Clin Invest. 1998 May 1; 101(9):1851-9. doi: 10.1172/JC11753).

Preferably, the ATP-hydrolyzing enzyme is soluble (secreted), i.e. not bound or attached to a (plasma) membrane. Without being bound to any theory, the present inventors assume that soluble ATP-hydrolyzing enzymes can reach various places (e.g., in the body) more efficiently as compared to membrane-bound enzymes. In particular, without being bound to any theory, it is assumed that the ATP-hydrolyzing enzyme mediates its beneficial effects (when combined with a checkpoint inhibitor) in the intestinal lumen, namely, by degrading extracellular ATP released from microbiota in the gut. The experimental data of this specification demonstrate the crucial role of the ATP-hydrolyzing enzyme on the ATP released from microbiota in the gut in order to mediate its beneficial effects on the activity of the checkpoint inhibitor. As membrane-bound ATP-hydrolyzing enzymes, such as endogenous CD39, cannot affect (the majority of) the extracellular ATP released by the microbiota in the gut, due to their limited activity range in the tissue where they are located, the ATP hydrolyzing enzyme is preferably not bound or attached to a (plasma) membrane. Accordingly, the ATP-hydrolyzing enzyme is preferably a soluble ATP-hydrolyzing enzyme.

Examples of soluble ATP-hydrolyzing enzymes include bacterial (e.g., Shigella flexneri) and potato apyrase as well as (engineered) soluble CD39.

Preferably, the ATP-hydrolyzing enzyme is apyrase. Apyrases are ATP-diphosphohydrolases that catalyze the sequential hydrolysis of ATP to ADP and ADP to AMP releasing inorganic phosphate. In particular, apyrases can also act on ADP and other nucleoside triphosphates and diphosphates in addition to ATP. Apyrase can be found in various eukaryotes in membrane bound and/or secreted soluble forms.

In general, the apyrase may have the sequence of any naturally occurring apyrase from any organism. In some embodiments, the apyrase is not an endogenous apyrase. In other words, the apyrase differs from the endogenous apyrase of the organism, to which it is administered. In certain embodiments, the apyrase is not a human endogenous apyrase, e.g. the apyrase may be a non-human apyrase. In some embodiments, the apyrase is not a mammalian apyrase. Preferably, the apyrase may be a bacterial or plant apyrase. For example, the apyrase may be Shigella flexneri apyrase or Solanum tuberosum (potato) apyrase. Moreover, the apyrase may be sequence variant of a naturally occurring apyrase exhibiting at least 50% or 60%, preferably at least 70% or 75%, more preferably at least 80% or 85%, even more preferably at least 90% or 95%, still more preferably at least 97% or 98%, such as at least 99% sequence identity to a naturally occurring apyrase. In particular, such a sequence variant may be functional, i.e., the ATP-hydrolyzing function of the apyrase is maintained in the sequence variant. The skilled person is aware of various bioinfomatics tools providing annotated sequences of proteins, including apyrases, and identifying active sites, domains and regions (such as nucleotide binding regions) important for the ATP-hydrolyzing functionality of a certain apyrase. Accordingly, the skilled person is well-aware, which amino acid positions must be maintained in an apyrase to maintain its ATP-hydrolyzing functionality. Preferably, the apyrase comprises the amino acid sequence of SEQ ID NO: 1. Also included are functional sequence variants of SEQ ID NO: 1 as described above, i.e. having at least 50% or 60%, preferably at least 70% or 75%, more preferably at least 80% or 85%, even more preferably at least 90% or 95%, still more preferably at least 97% or 98%, such as at least 99% sequence identity to a naturally occurring apyrase. In sequence variants of SEQ ID NO: 1 R192 must be maintained to ensure functionality.

The ATP-hydrolyzing enzyme may be obtained by any means. Preferably, the ATP-hydrolyzing enzyme is recombinantly produced. Preferably, the ATP-hydrolyzing enzyme is recombinantly produced apyrase. Preferably, the apyrase is recombinantly produced apyrase having the sequence of SEQ ID NO: 1 or a sequence variant thereof as described above, e.g. having at least 70% or 75%, more preferably at least 80% or 85%, even more preferably at least 90% or 95%, still more preferably at least 97% or 98%, such as at least 99% sequence identity; wherein R192 is preferably maintained. For recombinant production, the ATP-hydrolyzing enzyme may be encoded by a nucleic acid not naturally occurring in the cell or organism expressing the ATP-hydrolyzing enzyme. Recombinant production of the ATP-hydrolyzing enzyme may be achieved, for example, (1) by heterologous expression (wherein the apyrase sequence is derived from a different organism than the organism used for its expression), (2) by expression based on an expression vector (not occurring in nature; e.g. for overexpression of the ATP-hydrolyzing enzyme), (3) by not naturally occurring ATP-hydrolyzing enzymes (e.g., functional sequence variants as described above), or by any combination of (1)-(3). For example, a (heterologous) cell expressing the ATP-hydrolyzing enzyme may impart a post-translational modification (PTM; e.g., glycosylation) on the ATP-hydrolyzing enzyme that is not present in its native state. Such a PTM may result in a functional difference (e.g., reduced immunogenicity). Accordingly, the ATP-hydrolyzing enzyme may have a post-translational modification, which is distinct from the naturally produced ATP-hydrolyzing enzyme. As an alternative, the apyrase may be used directly from a natural source. The apyrase may be obtained from a plant source, an animal source or a bacterial source. The apyrase may be purified or cell extracts (such as periplasmic extracts of bacterial cells) may be used.

While the ATP-hydrolyzing enzyme may be used as protein/polypeptide, the ATP-hydrolyzing enzyme as described herein may also be encoded by a polynucleotide comprised in a nucleic acid. Accordingly, the present invention also provides a combination of (i) an immune checkpoint modulator and (ii) a nucleic acid molecule comprising a polynucleotide encoding the ATP-hydrolyzing enzyme as described herein. A nucleic acid (molecule) is a molecule comprising nucleic acid components. The term nucleic acid molecule usually refers to DNA or RNA molecules. It may be used synonymous with the term “polynucleotide”, i.e. the nucleic acid molecule may consist of a polynucleotide encoding the ATP-hydrolyzing enzyme. Alternatively, the nucleic acid molecule may also comprise further elements in addition to the polynucleotide encoding the ATP-hydrolyzing enzyme. Typically, a nucleic acid molecule is a polymer comprising or consisting of nucleotide monomers which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA or RNA molecules. Examples of nucleic acid molecules and/or polynucleotides include, e.g., a recombinant polynucleotide, a vector, an oligonucleotide, an RNA molecule such as an rRNA, an mRNA, an miRNA, an siRNA, or a tRNA, or a DNA molecule such as a cDNA.

Due to the redundancy of the genetic code, the present invention also comprises sequence variants of nucleic acid sequences, which encode the same amino acid sequences. For example, the polynucleotide encoding the apyrase having the amino acid sequence of SEQ ID NO: 1 may have the nucleotide sequence of SEQ ID NO: 3 or a sequence variant thereof encoding the same amino acid sequence of SEQ ID NO: 1 (due to the redundancy of the genetic code).

The polynucleotide encoding the ATP-hydrolyzing enzyme (or the complete nucleic acid molecule) may be optimized for expression of the ATP-hydrolyzing enzyme. For example, codon optimization of the nucleotide sequence may be used to improve the efficiency of translation in expression systems for the production of the ATP-hydrolyzing enzyme. Accordingly, the polynucleotide encoding of the ATP-hydrolyzing enzyme may be codon-optimized. The skilled artisan is aware of various tools for codon optimization, such as those described in: Ju Xin Chin, Bevan Kai-Sheng Chung, Dong-Yup Lee, Codon Optimization OnLine (COOL): a web-based multi-objective optimization platform for synthetic gene design, Bioinformatics, Volume 30, Issue 15, 1 Aug. 2014, Pages 2210-2212; or in: Grote A, Hiller K, Scheer M, Munch R, Nortemann B, Hempel D C, Jahn D, JCat: a novel tool to adapt codon usage of a target gene to its potential expression host. Nucleic Acids Res. 2005 Jul. 1; 33(Web Server issue):W526-31; or, forexample, Genscript's OptimumGene™ algorithm (as described in US 2011/0081708 A1).

Moreover, the nucleic acid molecule may comprise heterologous elements (i.e., elements, which in nature do not occur on the same nucleic acid molecule as the coding sequence for the ATP-hydrolyzing enzyme), e.g. for expression (such as heterologous expression) of the ATP-hydrolyzing enzyme. For example, a nucleic acid molecule may comprise a heterologous promoter, a heterologous enhancer, a heterologous UTR (e.g., for optimal translation/expression), a heterologous Poly-A-tail, and the like. In some embodiments the nucleic acid molecule may comprise an element conferring resistance against an antibiotic. In other embodiments, the nucleic acid molecule does not comprise an element conferring resistance against an antibiotic.

In general, the nucleic acid molecule may be manipulated to insert, delete or alter certain nucleic acid sequences. Changes from such manipulation include, but are not limited to, changes to introduce restriction sites, to amend codon usage, to add or optimize transcription and/or translation regulatory sequences, etc. It is also possible to change the nucleic acid to alter the encoded amino acids. For example, it may be useful to introduce one or more (e.g., 1, 2, 3, 4, 5, 6, 7, 8, 9, 10, etc.) amino acid substitutions, deletions and/or insertions into the amino acid sequence of the ATP-hydrolyzing enzyme. Such point mutations can modify stability, post-translational modifications, expression yield, etc.; can introduce amino acids for the attachment of covalent groups (e.g., labels); or can introduce tags (e.g., for purification purposes). Alternatively, a mutation in a nucleic acid sequence may be “silent”, i.e. not reflected in the amino acid sequence due to the redundancy of the genetic code as described above. In general, mutations can be introduced in specific sites or can be introduced at random, followed by selection (e.g., molecular evolution). For instance, a nucleic acid encoding the ATP-hydrolyzing enzyme can be randomly or directionally mutated to introduce different properties in the encoded amino acids. Such changes can be the result of an iterative process wherein initial changes are retained and new changes at other nucleotide positions are introduced. Further, changes achieved in independent steps may be combined.

In some embodiments, the nucleic acid molecule comprising a polynucleotide encoding the ATP-hydrolyzing enzyme may be a vector, for example an expression vector. A vector is usually a recombinant nucleic acid molecule, i.e. a nucleic acid molecule which does not occur in nature. Accordingly, the vector may comprise heterologous elements (i.e., sequence elements of different origin in nature). For example, the vector may comprise a multi cloning site, a heterologous promoter, a heterologous enhancer, a heterologous selection marker (to identify cells comprising said vector in comparison to cells not comprising said vector) and the like. A vector in the context of the present invention is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to the ATP-hydrolyzing enzyme. An expression vector may be used for production of expression products such as RNA, e.g. mRNA, or peptides, polypeptides or proteins. For example, an expression vector may comprise sequences needed for transcription of a sequence stretch of the vector, such as a (heterologous) promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present invention may be, e.g., an RNA vector or a DNA vector. For example, a vector in the sense of the present application may comprise a cloning site, a selection marker, and a sequence suitable for multiplication of the vector, such as an origin of replication. A vector in the context of the present application may be a plasmid vector.

In some embodiments, the vector is an expression vector. Expression vectors may be capable of enhancing the expression of one or more polynucleotides that have been inserted or cloned into the vector. Examples of such expression vectors include, bacteriophages, autonomously replicating sequences (ARS), centromeres, and other sequences which are able to replicate or be replicated in vitro or in a cell, or to convey a nucleic acid segment to a particular location within a cell of an animal or human. Expression vectors useful in the present invention include chromosomal-, episomal- and virus-derived vectors, e.g., vectors derived from bacterial plasmids or bacteriophages, and vectors derived from combinations thereof, such as cosmids and phagemids or virus-based vectors such as adenovirus, AAV, lentiviruses.

The expression vector may be a plasmid. Any plasmid expression vector may be used provided that it is replicable and viable in the host.

For expression of the ATP-hydrolyzing enzyme in bacteria, the expression vector is preferably a vector optimized for protein expression in bacteria, e.g. in E. coli. Such expression vectors are well-known in the art and commercially available. For example, the pBAD vector system may be used, which provides a reliable and controllable system for expressing recombinant proteins in bacteria. This system is based on the araBAD operon, which controls E. coli L-arabinose metabolism. The polynucleotide encoding the ATP-hydrolyzing enzyme may be placed into the pBAD vector downstream of the araBAD promoter, which then drives expression of the ATP-hydrolyzing enzyme in response to L-arabinose, and is inhibited by glucose.

In some embodiments, the expression vector may be mini-circle DNA. Mini-circle DNA are useful for persistently high levels of nucleic acid transcription. The circular vectors are characterized by being devoid of expression-silencing bacterial sequences. For example, mini-circle vectors differ from bacterial plasmid vectors in that they lack an origin of replication, and lack drug selection markers commonly found in bacterial plasmids, e.g. β-lactamase, tet, and the like. Consequently, minicircle DNA becomes smaller in size, allowing more efficient delivery.

In certain embodiments, the expression vector may be a viral vector. Any viral vector based on any virus may be used as a carrier for the agent. Commonly used classes of viral systems used in gene therapy can be categorized into two groups according to whether their genomes integrate into host cellular chromatin (oncoretroviruses and lentiviruses) or persist in the cell nucleus predominantly as extrachromosomal episomes (adeno-associated viruses, adenoviruses and herpesviruses). Accordingly, the viral vector may be a retroviral, lentiviral, adenoviral, herpesviral or adeno-associated viral vector, as described below. Moreover, the viral vector may be derived from any of retroviruses, lentiviruses, adeno-associated viruses, adenoviruses or herpesviruses.

The viral vector may be an adenoviral (AdV) vector. Adenoviruses are medium-sized double-stranded, non-enveloped DNA viruses with linear genomes that is between 26-48 Kbp. Adenoviruses gain entry to a target cell by receptor-mediated binding and internalization, penetrating the nucleus in both non-dividing and dividing cells. Adenoviruses are heavily reliant on the host cell for survival and replication and are able to replicate in the nucleus of vertebrate cells using the host's replication machinery.

The viral vector may be from the Parvoviridae family. The Parvoviridae is a family of small single-stranded, non-enveloped DNA viruses with genomes approximately 5000 nucleotides long. The viral vector may be an adeno-associated virus (AAV). AAV is a dependent parvovirus that generally requires co-infection with another virus (typically an adenovirus or herpesvirus) to initiate and sustain a productive infectious cycle. In the absence of such a helper virus, AAV is still competent to infect or transduce a target cell by receptor-mediated binding and internalization, penetrating the nucleus in both non-dividing and dividing cells. Because progeny virus is not produced from AAV infection in the absence of helper virus, the extent of transduction is restricted only to the initial cells that are infected with the virus. Unlike retrovirus, adenovirus, and herpes simplex virus, AAV appears to lack human pathogenicity and toxicity.

Viral vectors based on viruses from the family Retroviridae may be used. Retroviruses comprise single-stranded RNA animal viruses that are characterized by two unique features. First, the genome of a retrovirus is diploid, consisting of two copies of the RNA. Second, this RNA is transcribed by the virion-associated enzyme reverse transcriptase into double-stranded DNA. This double-stranded DNA or provirus can then integrate into the host genome and be passed from parent cell to progeny cells as a stably-integrated component of the host genome.

Preferably, the expression vector is a plasmid. As an alternative, preferably the expression vector is a bacteriophage. Where the expression vector is a plasmid or a bacteriophage, the expression vector may be transformed into a bacterial cell and the bacterial cell included in the composition of the invention. The bacterial cell may be E. coli. As an alternative the bacterial carrier may be attenuated Salmonella enterica. The attenuated Salmonella enterica may be of the serovar Salmonella Typhimurium.

In some embodiments, the nucleic acid molecule comprising a polynucleotide encoding the ATP-hydrolyzing enzyme as described herein may be a genomic nucleic acid molecule, for example genomic DNA (e.g. chromosomal DNA). In other words, the polynucleotide encoding the ATP-hydrolyzing enzyme may be integrated into the genome (of an organism (heterologously) expressing the ATP-hydrolyzing enzyme.

In some embodiments, a DNA fragment may be introduced into, e.g., a host cell/microorganism, such as a bacterium, for integration into the genome of the host cell/microorganism, such as a bacterium. To this end, the DNA fragment may contain a nucleotide sequence encoding the ATP-hydrolyzing enzyme, in particular an apyrase, as described herein (for example the S. flexneri phoN2 gene) for the integration into the genome, e.g. of a host cell/microorganism, such as a bacterium. For example, such a DNA fragment may be for the integration of S. flexneri phoN2 gene in E. coli Nissle (EcN) genome. An exemplified DNA fragment for the integration of S. flexneri phoN2 gene in E. coli Nissle (EcN) genome is shown in FIG. 39. In some embodiments, the DNA fragment may contain malP: EcN gene for maltodextrin phosphorylase; cat: E. coli gene for chloramphenicol acetyltransferase; phoN2: S. flexneri gene for apyrase; malT: EcN gene for the transcriptional activator of the maltose and maltodextrins operon; FRT: Flippase Recognition Target sequence; P_{cat}: promoter of the cat gene; P_{proD}: promoter of the phoN2 gene; BBa_BB0032 RBS: Ribosome Binding Site of the phoN2 gene; and/or T_{phoN2}: transcriptional terminator of the phoN2 gene. In some embodiments, the nucleotide sequence of the EcN malP gene portion is according to SEQ ID NO: 4 or a sequence variant thereof having at least 75%, 80%, 85%, 90% or 95% sequence identity. In some embodiments, the nucleotide sequence of the EcN malT gene portion is according to SEQ ID NO: 5 or a sequence variant thereof having at least 75%, 80%, 85%, 90% or 95% sequence identity. In some embodiments, the DNA fragment including the P_{proD} promoter, the BBa_BB0032 RBS, the S. flexneri phoN2 gene and the phoN2 transcriptional terminator may be according to SEQ ID NO: 6 or a sequence variant thereof having at least 75%, 80%, 85%, 90% or 95% sequence identity. In some embodiments, the DNA fragment including the E. coli cat gene flanked by the FRT sequences may be according to SEQ ID NO: 7 or a sequence variant thereof having at least 75%, 80%, 85%, 90% or 95% sequence identity.

### Host Cells, Microorganisms and Viral Particles

In a further aspect, the present invention also provides a combination of (i) an immune checkpoint modulator and (ii) a host cell comprising the nucleic acid molecule as described herein, i.e. the nucleic acid comprising the polynucleotide encoding the ATP-hydrolyzing enzyme as described herein.

Host cells may be prokaryotic or eukaryotic cells. Examples of such cells include but are not limited to, eukaryotic cells, e.g., yeast cells, animal cells or plant cells or prokaryotic cells, including E. coli. In some embodiments, the cells may be mammalian cells, such as a mammalian cell line. Examples include human cells, CHO cells, HEK293T cells, PER.C6 cells, NS0 cells, human liver cells, or myeloma cells.

The cell may be transformed or transfected with a nucleic acid, such as a (expression) vector, as described above. The term “transfection” refers to the introduction of nucleic acid molecules, such as DNA or RNA molecules (e.g. plasmids), into eukaryotic animal/human cells, while the term “transformation” usually refers to the introduction of nucleic acid molecules, such as DNA or RNA molecules (e.g. plasmids), into bacterial cells, yeast cells, plant cells or fungi cells. In the context of the present invention, the terms “transfection” and “transformation” encompass any method known to the skilled person for introducing nucleic acid molecules into cells, such as into mammalian or bacterial cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. In some embodiments, the introduction is non-viral. For bacterial cells, competent bacteria may be used for transformation.

Moreover, the cells of the present invention may be transfected/transformed stably or transiently with the nucleic acid (vector), e.g. for expressing the ATP-hydrolyzing enzyme as described herein. In some embodiments, the cells are stably transfected with the nucleic acid (vector) comprising a polynucleotide encoding the ATP-hydrolyzing enzyme as described herein. In other embodiments, the cells are transiently transfected/transformed with the nucleic acid (vector) comprising a polynucleotide encoding the ATP-hydrolyzing enzyme as described herein.

Accordingly, the present invention also provides a combination of (i) an immune checkpoint modulator and (ii) a recombinant host cell, which heterologously expresses the ATP-hydrolyzing enzyme as described herein. For example, the cell may be of another species than the ATP-hydrolyzing enzyme. In some embodiments, the cell type of the cell does not express (such) an ATP-hydrolyzing enzyme in nature. Moreover, the host cell may impart a post-translational modification (PTM; e.g., glycosylation) on the ATP-hydrolyzing enzyme that is not present in their native state. Such a PTM may result in a functional difference (e.g., reduced immunogenicity). Accordingly, the ATP-hydrolyzing enzyme may have a post-translational modification, which is distinct from the naturally produced ATP-hydrolyzing enzyme.

In a further aspect, the present invention also provides a combination of (i) an immune checkpoint modulator and (ii) a microorganism comprising the nucleic acid molecule as described herein, i.e. the nucleic acid comprising the polynucleotide encoding the ATP-hydrolyzing enzyme as described herein. The microorganism may be a live microorganism.

As used herein, the term “microorganism” refers to a microscopic organism, which may exist in its single-celled form or in a colony of cells. Typically, the term “microorganism” includes all unicellular organisms. Accordingly, the microorganism may be selected from prokaryotes, such as archea and bacteria, and eukaryotes, such as unicellular protists, protozoans, fungi and plants.

Preferably, the microorganism is a prokaryotic microorganism, such as a bacterium, or a eukaryotic microorganism, such as a yeast. In some embodiments, the microorganism is selected from the group consisting of Escherichia spp., Salmonella spp., Yersinia spp., Vibrio spp., Listeria spp., Lactococcus spp., Shigella spp., Cyanobacteria, and Saccharomyces spp. As used herein, the expression “spp.” in connection with any microorganism is intended to comprise all members of a given genus, including species, subspecies and others.

In certain embodiments, the microorganisms may be provided as probiotics (e.g., of live bacteria). As used herein, the term “probiotics” refers to live microorganisms, such as bacteria or yeasts, providing health benefits when consumed, for example by improving or restoring the gut flora. Such live microorganisms can be used as food additive due to the health benefits they can provide. Those can be for example lyophilized in granules, pills or capsules, or directly mixed with dairy products for consumption. Examples of microorganisms, for which health benefits have been demonstrated include, but are not limited to Lactobacillus, Bifidobacterium, Saccharomyces, Lactococcus, Enterococcus, Streptococcus, Pediococcus, Leuconostoc, Bacillus, Escherichia coli, in particular regarding probiotic strains thereof, such as those described in Fijan S. Microorganisms with claimed probiotic properties: an overview of recent literature. Int J Environ Res Public Health. 2014; 11(5):4745-4767. doi:10.3390/ijerph110504745, which is incorporated herein by reference.

In case of virulent microorganisms, the virulence of the microorganism may be attenuated. Methods for attenuating the virulence, e.g. of bacteria, are known in the art and described, for example, in WO 2018/089841. In general, attenuation of virulence may be achieved by a mutation of a virulence factor from a virulent pathogen.

In particular, the present invention provides a combination of (i) an immune checkpoint modulator and (ii) a bacterium (bacterial cell) comprising the nucleic acid molecule as described herein, i.e. the nucleic acid comprising the polynucleotide encoding the ATP-hydrolyzing enzyme as described herein. Accordingly, the host cell as described above may be a bacterial cell and the microorganism as described above may be a bacterium.

The bacterium may be a recombinant bacterium, i.e. a bacterium, which does not occur in nature. In particular, the recombinant bacterium may comprise nucleic acid sequences not occurring in the bacterium in nature, e.g. for heterologous expression or overexpression of the ATP-hydrolyzing enzyme. Accordingly, the bacterium may heterologously express the ATP-hydrolyzing enzyme (i.e., the expressed ATP-hydrolyzing enzyme may not naturally occur in the bacterium and may be derived from a distinct strain, species etc.); or the bacterium may overexpress the ATP-hydrolyzing enzyme. As used herein, the term “overexpression” refers to artificial expression of a gene of interest (e.g. encoding the ATP-hydrolyzing enzyme) in increased quantity. Overexpression can be achieved by various ways, e.g. by increasing the number of nucleic acid molecules encoding the gene of interest (e.g. encoding the ATP-hydrolyzing enzyme) and/or by the use of regulatory elements increasing expression (e.g. promoters, enhancers or other gene-regulatory elements).

The bacterium may be a live bacterium. If the bacterium is a pathogen, its virulence may be attenuated as described above. In general, the bacterium may be selected from Gram-positive or Gram-negative bacteria. In some embodiments, the bacterium may be a Gram-negative bacterium, such as a bacterium selected from Escherichia spp., Salmonella spp., Yersinia spp., Vibrio spp., Shigella spp., or Cyanobacteria, such as a bacterium selected from Escherichia coli, Salmonella typhi, Salmonella typhimurium, Yersinia enterocolitica, Vibrio cholerae, and Shigella flexneri. In some embodiments, the bacterium may be a Gram-positive bacterium. Examples of Gram-positive bacteria include Lactococcus spp., such as Lactococcus lactis, and Listeria spp., such as Listeria monocytogenes. Preferably, the bacterium may be Escherichia coli, Lactococcus lactis or Salmonella typhimurium. Particularly preferably, the bacterium may be Escherichia coli, Lactococcus lactis or Salmonella typhimurium, in particular (heterologously) expressing apyrase.

The bacterium may provide probiotic properties, as described above. In particular, the probiotic bacterium may be Lactococcus lactis or a probiotic strain of Escherichia coli, such as Escherichia coli Nissle 1917 (EcN). Escherichia coli Nissle 1917 was shown to treat constipation (Chmielewska A., Szajewska H. Systematic review of randomised controlled trials: Probiotics for functional constipation. World J. Gastroenterol. 2010; 16:69-75) and inflammatory bowel disease (Behnsen J., Deriu E., Sassone-Corsi M., Raffatellu M. Probiotics: Properties, examples, and specific applications. Cold Spring Harb. Perspect. Med. 2013; 3 doi: 10.1101/cshperspect.a010074) and to relieve gastrointestinal disorder, ulcerative colitis, and Crohn's disease (Xia P., Zhu J., Zhu G. Escherichia coli Nissle 1917 as safe vehicles for intestinal immune targeted therapy—A review. Acta Microbiol. Sin. 2013; 53:538-544).

In a further aspect, the present invention also provides a combination of (i) an immune checkpoint modulator and (ii) a viral particle comprising the nucleic acid molecule as described herein, i.e. the nucleic acid comprising the polynucleotide encoding the ATP-hydrolyzing enzyme as described herein. As used herein, the term “viral particle” includes virions as well as virus-like particles. A “virion” (“virus”) is a structure, which can usually transfer nucleic acid from one cell to another, and may be “enveloped” or “non-enveloped”. As used herein, a “virus-like particle” (also “VLP”) refers in particular to a non-replicating, viral shell, derived from any of several viruses. VLPs lack the viral components that are required for virus replication and thus represent a highly attenuated form of a virus. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. Virus like particles and methods of their production are known and familiar to the person of ordinary skill in the art, and viral proteins from several viruses are known to form VLPs, including human papillomavirus, HIV (Kang et al., Biol. Chem. 380: 353-64 (1999)), Semliki-Forest virus (Notka et al., Biol. Chem. 380: 341-52 (1999)), human polyomavirus (Goldmann et al., J. Virol. 73: 4465-9 (1999)), rota virus (Jiang et al., Vaccine 17: 1005-13 (1999)), parvovirus (Casal, Biotechnology and Applied Biochemistry, Vol 29, Part 2, pp 141-150 (1999)), canine parvovirus (Hurtado et al., J. Viral. 70: 5422-9 (1996)), hepatitis E virus (Li et al., J. Viral. 71: 35 7207-13 (1997)), and Newcastle disease virus. The formation of such VLPs can be detected by any suitable technique. Examples of suitable techniques known in the art for detection of VLPs in a medium include, e.g., electron microscopy techniques, dynamic light scattering (DLS), selective chromatographic separation (e.g., ion exchange, hydrophobic interaction, and/or size exclusion chromatographic separation of the VLPs) and density gradient centrifugation. Further, VLPs can be isolated by known techniques, e.g., density gradient centrifugation and identified by characteristic density banding. See, for example, Baker et al. (1991) Biophys. J. 60: 1445-1456; and Hagensee et al. (1994) J. Viral. 68:4503-4505; Vincente, J Invertebr Pathol., 2011; Schneider-Ohrum and Ross, Curr. Top. Microbial. Immunol., 354: 53073 (2012).

Preferably, the viral particle is not infectious in humans. In particular, viruses infecting and replicating in bacteria, such as bacteriophages, may be used. Accordingly, the present invention also provides a combination of (i) an immune checkpoint modulator and (ii) a bacteriophage comprising the nucleic acid molecule as described herein, i.e. the nucleic acid comprising the polynucleotide encoding the ATP-hydrolyzing enzyme as described herein. A bacteriophage is a virus that infects and replicates within bacteria and archaea. Bacteriophages are usually composed of proteins that encapsulate a DNA or RNA genome, and occur in various distinct structures, that may be either simple or elaborate. Phages may provide antibacterial effects. Bacteriophages comprising the nucleic acid comprising the polynucleotide encoding the ATP-hydrolyzing enzyme may readily transfer the nucleic acid comprising the polynucleotide encoding the ATP-hydrolyzing enzyme to bacteria, such that the ATP-hydrolyzing enzyme is expressed by bacteria.

### Compositions

The immune checkpoint modulator of the combinations of the invention as described above may be provided in a composition. Accordingly, each of the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, and the viral particle comprising the nucleic acid comprising the polynucleotide encoding the ATP hydrolyzing enzyme may be provided in a composition. The composition may be a vaccine.

For example, the composition may be a pharmaceutical composition, which may optionally comprise a pharmaceutically acceptable carrier, diluent and/or excipient. Although the carrier, diluent or excipient may facilitate administration, it should not itself be harmful to the individual receiving the composition. Nor should it be toxic. Usually, carriers, diluents and excipients are not “active” components of the composition. Accordingly, the immune checkpoint modulator, the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme may be the sole active component of the composition (i.e. which is pharmaceutically active, in particular with regard to the disease to be treated). Suitable carriers may be large, slowly metabolized macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates.

The composition may comprise a vehicle. A vehicle is typically understood to be a material that is suitable for storing, transporting, and/or administering a compound, such as a pharmaceutically active compound. For example, the vehicle may be a physiologically acceptable liquid, which is suitable for storing, transporting, and/or administering a pharmaceutically active compound. Once formulated, the compositions can be administered directly to the subject. In some embodiments the compositions are adapted for administration to mammalian, e.g., human subjects.

In some embodiments, the pharmaceutical composition may include an antimicrobial, particularly if packaged in a multiple dose format. They may comprise detergent e.g., a Tween (polysorbate), such as Tween 80. Detergents are generally present at low levels e.g., less than 0.01%. Compositions may also include sodium salts (e.g., sodium chloride) to give tonicity. For example, a concentration of 10±2 mg/ml NaCl is typical.

Further, pharmaceutical compositions may comprise a sugar alcohol (e.g., mannitol) or a disaccharide (e.g., sucrose or trehalose) e.g., at around 15-30 mg/ml (e.g., 25 mg/ml), particularly if they are to be lyophilized or if they include material which has been reconstituted from lyophilized material. The pH of a composition for lyophilization may be adjusted to between 5 and 8, or between 5.5 and 7, or around 6.1 prior to lyophilization.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject. A thorough discussion of pharmaceutically acceptable carriers is available in Gennaro (2000) Remington: The Science and Practice of Pharmacy, 20th edition, ISBN: 0683306472.

Pharmaceutical compositions may be prepared in various forms and may be administered by any number of routes including, but not limited to, oral, intravenous, intramuscular, intraarterial, intraperitoneal, subcutaneous, enteral, sublingual, or rectal routes. Preferably, the pharmaceutical composition may be prepared for oral administration, e.g. as tablets, capsules and the like, or as injectable, e.g. as liquid solutions or suspensions. In some embodiments, the pharmaceutical composition is an injectable. Solid forms suitable for solution in, or suspension in, liquid vehicles prior to injection are also encompassed, for example the pharmaceutical composition may be in lyophilized form.

The composition may be prepared for oral administration e.g., as a tablet or capsule, as a spray, or as a syrup (optionally flavored). Orally acceptable dosage forms include, but are not limited to, capsules, tablets, aqueous suspensions or solutions. In the case of tablets for oral use, carriers commonly used may include lactose and corn starch. Lubricating agents, such as magnesium stearate, may also be added. For oral administration in a capsule form, useful diluents include lactose and dried cornstarch. When aqueous suspensions are required for oral use, the active ingredient, i.e. the immune checkpoint modulator, the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme, may be combined with emulsifying and suspending agents. If desired, certain sweetening, flavoring or coloring agents may also be added. As such, the active component may be susceptible to degradation in the gastrointestinal tract. Thus, if the composition is to be administered by a route using the gastrointestinal tract, the composition may contain agents which protect the ATP-hydrolyzing enzyme or the checkpoint modulator from degradation but which release the ATP-hydrolyzing enzyme or the checkpoint modulator once it has been absorbed from the gastrointestinal tract. The composition may be in kit form, designed such that a combined composition is reconstituted just prior to administration to a subject. For example, a lyophilized ATP-hydrolyzing enzyme or immune checkpoint inhibitor may be provided in kit form with sterile water or a sterile buffer.

Within the scope of the invention are compositions present in several forms adapted for various routes of administration; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the ATP-hydrolyzing enzyme or the checkpoint modulator may be in dry form, for reconstitution before use with an appropriate sterile liquid. In some embodiments, the compositions may be prepared as injectables, either as liquid solutions or suspensions. Solid forms suitable for solution in, or suspension in, liquid vehicles prior to injection can also be prepared (e.g., a lyophilized composition, e.g. for reconstitution with sterile water containing a preservative). For injection, e.g. intravenous, cutaneous or subcutaneous injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection, lactated Ringer's injection. Preservatives, stabilizers, buffers, antioxidants and/or other additives may be included, as required. For injection, the pharmaceutical composition may be provided, for example, in a pre-filled syringe.

Pharmaceutical compositions may generally have a pH between 5.5 and 8.5, in some embodiments this may be between 6 and 8, for example about 7. The pH may be maintained by the use of a buffer. The composition may be sterile and/or pyrogen free. The composition may be gluten free. The composition may be isotonic with respect to humans. In some embodiments pharmaceutical compositions may be supplied in hermetically-sealed containers.

Whether it is a protein, a peptide, a nucleic acid molecule, a host cell, a microorganism, a viral particle or another pharmaceutically useful compound as described above that is to be given to an individual, administration is usually in a “prophylactically effective amount” or in a “therapeutically effective amount” (as the case may be), this being sufficient to show benefit to the individual. The actual amount administered, and rate and time-course of administration, will depend on the nature and severity of what is being treated. Accordingly, an “effective” amount of one or more active ingredients is usually an amount that is sufficient to treat, ameliorate, attenuate, reduce or prevent a desired disease or condition, or to exhibit a detectable therapeutic effect. Therapeutic effects also include reduction or attenuation in pathogenic potency or physical symptoms. The precise effective amount for any particular subject will depend upon their size, weight, and health, the nature and extent of the condition, and the therapeutics or combination of therapeutics selected for administration. The effective amount for a given situation is determined by routine experimentation and is within the judgment of a clinician.

In certain embodiments, the pharmaceutical composition according to the present invention may also comprise an additional active component, which is not the immune checkpoint modulator, the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme. The additional active component is typically pharmaceutically active with regard to the same disease, for example cancer. For the treatment of cancer, examples of additionally active compounds include, but are not limited to, an anti-cancer agent (such as a cytostatic agent) or an antibody directed against a tumor-associated or tumor-specific antigen. Accordingly, the pharmaceutical composition according to the present invention may comprise one or more of the additional active components.

The (i) immune checkpoint modulator, and (ii) the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme, can be present either in the same pharmaceutical composition as the additional active component or, alternatively, comprised in a separate pharmaceutical composition. Accordingly, each additional active component may be comprised in a distinct pharmaceutical composition. Preferably, components (i) and (ii); i.e. (i) the immune checkpoint modulator, and (ii) the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme; may be comprised in distinct (pharmaceutical) compositions. Such different pharmaceutical compositions may be administered either combined/simultaneously or at separate times or at separate locations (e.g., separate parts of the body) or via distinct routes of administration. For example, (the composition comprising) the immune checkpoint modulator may be administered via a parenteral route of administration, while (the composition comprising) the ATP hydrolyzing enzyme, the nucleic acid comprising the polynucleotide encoding the ATP hydrolyzing enzyme, the host cell, the microorganism, or the viral particle comprising the nucleic acid may be administered via an enteral route of administration.

In certain embodiments, the immune checkpoint modulator or the ATP hydrolyzing enzyme may make up at least 50% by weight (e.g., 60%, 70%, 75%, 80%, 85%, 90%, 95%, 96%, 97%, 98%, 99% or more) of the total protein in the composition.

In some embodiments, the composition may contain the immune checkpoint modulator, the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme in purified form.

In some instances, the composition may contain a cell extract comprising the ATP hydrolyzing enzyme or the nucleic acid comprising the polynucleotide encoding the ATP hydrolyzing enzyme. For example, the composition may comprise a cell extract from a cell expressing the ATP hydrolyzing enzyme or a cell comprising the nucleic acid comprising the polynucleotide encoding the ATP hydrolyzing enzyme. Such a cell may be a bacterial cell as described above. For example, the composition may comprise a periplasmic extract of a bacterium comprising the nucleic acid comprising the polynucleotide encoding the ATP hydrolyzing enzyme. Preferred bacteria (bacterial cells) in this context are those described above.

In some embodiments, the composition may be formulated for administration in a nanocapsule. Preferably, the composition comprising the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme; may be formulated for administration in a nanocapsule (while the composition comprising the immune checkpoint modulator may or may not be formulated for administration in a nanocapsule. Accordingly, the present invention also provides a nanocapsule comprising the composition as described herein. In particular, the present invention provides a nanocapsule comprising (a composition comprising) the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme.

A nanocapsule is usually made from a nontoxic polymer/lipid and can protect substances from adverse environment. Nanocapsules are usually vesicular systems made of a polymeric membrane which encapsulates an inner liquid core at the nanoscale. Encapsulation methods are known in the art and include nanoprecipitation, emulsion-diffusion and solvent-evaporation. In some embodiments, the nanocapsule may be for enteral, in particular oral, administration. Nanocapsules and methods for preparing nanocapsules are known in the art and described, for example, in Erdogar N, Akkin S, Bilensoy E. Nanocapsules for Drug Delivery: An Updated Review of the Last Decade. Recent Pat Drug Deliv Formul. 2018; 12(4):252-266. doi: 10.2174/1872211313666190123153711, which is incorporated herein in its entirety.

The present invention also provides a method of preparing a (pharmaceutical) composition comprising the steps of: (i) preparing the immune checkpoint modulator, the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme; and (ii) admixing it with one or more pharmaceutically acceptable carriers.

### Combinations

According to the present invention, the immune checkpoint modulator is combined with an ATP-hydrolyzing enzyme, a nucleic acid encoding the ATP-hydrolyzing enzyme, or a host cell, microorganism or viral particle comprising the nucleic acid encoding the ATP-hydrolyzing enzyme. The ATP-hydrolyzing enzyme encoded by the nucleic acid may be expressed, such that at the place of action (e.g., in the human or animal body), where the combination exerts its effects, the immune checkpoint modulator is combined with the ATP-hydrolyzing enzyme.

In general, a “combination” of (i) the immune checkpoint modulator as described herein and of (ii) the ATP-hydrolyzing enzyme, a nucleic acid encoding the ATP-hydrolyzing enzyme, or a host cell, microorganism or viral particle comprising the nucleic acid encoding the ATP-hydrolyzing enzyme, as described herein, means that both components can exert their effects in a combined manner. To this end, the time window of the effects of both components usually overlaps. Accordingly, the effects of both components are usually present in the human or animal body at the same time (even if one or both of the components may be no longer physically present). In some embodiments, both components may be (physically) present in the human or animal body at the same time.

Accordingly, (i) the treatment with the immune checkpoint modulator as described herein preferably overlaps with (ii) the treatment with the ATP-hydrolyzing enzyme, a nucleic acid encoding the ATP-hydrolyzing enzyme, or a host cell, microorganism or viral particle comprising the nucleic acid encoding the ATP-hydrolyzing enzyme, as described herein. Even if one component (i) or (ii) may not be administered, e.g., at the same day, as the other component (the other of (i) or (ii)), their treatment schedules are usually intertwined.

In some embodiments, (i) the immune checkpoint modulator as described herein and/or (ii) the ATP-hydrolyzing enzyme, a nucleic acid encoding the ATP-hydrolyzing enzyme, or a host cell, microorganism or viral particle comprising the nucleic acid encoding the ATP-hydrolyzing enzyme, as described herein, may be administered repeatedly. For example, the administration of (i) the immune checkpoint modulator as described herein may be followed by the administration of (ii) the ATP-hydrolyzing enzyme, a nucleic acid encoding the ATP-hydrolyzing enzyme, or a host cell, microorganism or viral particle comprising the nucleic acid encoding the ATP-hydrolyzing enzyme, as described herein, and, thereafter, a further administration of (i) the immune checkpoint modulator as described herein may follow. Similarly, the administration of (ii) the ATP-hydrolyzing enzyme, a nucleic acid encoding the ATP-hydrolyzing enzyme, or a host cell, microorganism or viral particle comprising the nucleic acid encoding the ATP-hydrolyzing enzyme, as described herein may be followed by the administration of (i) the immune checkpoint modulator as described herein, and, thereafter, a further administration of (ii) the ATP-hydrolyzing enzyme, a nucleic acid encoding the ATP-hydrolyzing enzyme, or a host cell, microorganism or viral particle comprising the nucleic acid encoding the ATP-hydrolyzing enzyme, as described herein may follow. In this way, the treatment schedules of both components (i) and (ii) may be intertwined.

The immune checkpoint modulator combined with the ATP-hydrolyzing enzyme, the nucleic acid encoding the ATP-hydrolyzing enzyme, or the host cell, microorganism or viral particle comprising the nucleic acid encoding the ATP-hydrolyzing enzyme may provide an additive therapeutic effect, such as a synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

In some embodiments, the combination of (i) the immune checkpoint modulator as described herein and (ii) the ATP-hydrolyzing enzyme, a nucleic acid encoding the ATP-hydrolyzing enzyme, or a host cell, microorganism or viral particle comprising the nucleic acid encoding the ATP-hydrolyzing enzyme, as described herein, may be combined with a further (“third”) component, such as an antigen or a fragment thereof comprising at least one epitope, a nucleic acid encoding the antigen or the fragment thereof, or a host cell, microorganism or viral particle comprising the nucleic acid encoding the antigen or the fragment thereof.

In other words, the combination of (i) the immune checkpoint modulator as described herein and (ii) the ATP-hydrolyzing enzyme, a nucleic acid encoding the ATP-hydrolyzing enzyme, or a host cell, microorganism or viral particle comprising the nucleic acid encoding the ATP-hydrolyzing enzyme, as described herein, may further comprise any one (or a combination) of:
(a) an antigen or a fragment thereof comprising at least one antigenic epitope,
(b) a nucleic acid comprising a polynucleotide encoding the antigen or the fragment thereof comprising at least one antigenic epitope,
(c) a host cell comprising the nucleic acid,
(d) a microorganism comprising the nucleic acid, or
(e) a viral particle comprising the nucleic acid.

As used herein, an “antigen” is any structural substance which serves as a target for the receptors of an adaptive immune response, in particular as a target for antibodies, T cell receptors, and/or B cell receptors. An “epitope”, also known as “antigenic determinant”, is the part (or fragment) of an antigen that is recognized by the immune system, in particular by antibodies, T cell receptors, and/or B cell receptors. Thus, one antigen comprises at least one epitope, i.e. a single antigen may have one or more epitopes. In the context of the present invention, the term “epitope” is mainly used to designate T cell epitopes, which are presented on the surface of an antigen-presenting cell, where they are bound to Major Histocompatibility Complex (MHC). T cell epitopes presented by MHC class I molecules are typically, but not exclusively, peptides between 8 and 11 amino acids in length, whereas MHC class II molecules present longer peptides, generally, but not exclusively, between 12 and 25 amino acids in length.

Preferably, the combination of (i) the immune checkpoint modulator as described herein and (ii) the ATP-hydrolyzing enzyme, a nucleic acid encoding the ATP-hydrolyzing enzyme, or a host cell, microorganism or viral particle comprising the nucleic acid encoding the ATP-hydrolyzing enzyme, as described herein, is further combined with a fragment of an antigen, said fragment comprising at least one epitope of said antigen. As used herein, a “fragment” of an antigen comprises at least 10 consecutive amino acids of the antigen, preferably at least 15 consecutive amino acids of the antigen, more preferably at least 20 consecutive amino acids of the antigen, even more preferably at least 25 consecutive amino acids of the antigen and most preferably at least 30 consecutive amino acids of the antigen.

Furthermore, a “sequence variant” of an antigen or a fragment thereof comprising at least one epitope may be used, which has an (amino acid) sequence which is at least 70% or at least 75%, preferably at least 80% or at least 85%, more preferably at least 90% or at least 95%, even more preferably at least 97% or at least 98%, particularly preferably at least 99% identical to the reference sequence (e.g., a naturally occurring antigen or fragment). A “functional” sequence variant is preferred and means in the context of an antigen/antigen fragment/epitope, that the function of the epitope(s), e.g. comprised by the antigen (fragment), is not impaired or abolished, i.e. that it is immunogenic, preferably has similar/the same immunogenicity as the epitope comprised in the full length antigen. In some embodiments, the amino acid sequence of the epitope(s), e.g. comprised by the cancer/tumor antigen (fragment) as described herein, is not mutated and, thus, identical to a (naturally occurring) reference epitope sequence.

The antigen is typically selected in view of the desired immune response elicited or enhanced by the combination of (i) the immune checkpoint modulator as described herein and (ii) the ATP-hydrolyzing enzyme, a nucleic acid encoding the ATP-hydrolyzing enzyme, or a host cell, microorganism or viral particle comprising the nucleic acid encoding the ATP-hydrolyzing enzyme, as described herein. In other words, the selected antigen (fragment) may determine the target/direction of the immune response elicited or enhanced by the combination of the invention as described herein.

For example, in the context of cancer/tumor, the antigen (or the fragment thereof) is a cancer/tumor antigen, in particular a cancer/tumor-associated antigen or a cancer/tumor-specific antigen. Many cancer/tumor antigens are known in the art to be associated with one or more particular cancer or tumor, such that—depending on the type of cancer/tumor and/or the desired treatment effect, an appropriate antigen or fragment thereof can be selected.

As used herein, “cancer/tumor antigens/epitopes” are antigens/epitopes produced by cancer/tumor cells. Such antigens/epitopes are typically specific for (or associated with) a certain kind of cancer/tumor.

Cancer/tumor-associated (also cancer/tumor-related) antigens (TAAs) are antigens, which are usually expressed by both, cancer/tumor cells and normal cells. For example, a TAA may be one or more surface proteins or polypeptides, nuclear proteins or glycoproteins, or fragments thereof, expressed by a tumor cell. For example, human tumor-associated antigens include differentiation antigens (such as melanocyte differentiation antigens), mutational antigens (such as p53), overexpressed cellular antigens (such as HER2), viral antigens (such as human papillomavirus proteins), and cancer/testis (CT) antigens that are expressed in germ cells of the testis and ovary but are silent in normal somatic cells (such as MAGE and NY-ESO-1). Many TAAs are not cancer- or tumor-specific and may also be found on normal tissues. Accordingly, those antigens may be present since birth (or even before). Therefore, there is a chance that the immune system developed self-tolerance to those antigens.

Cancer/tumor-specific antigens (TSAs), in contrast, are antigens, which are expressed specifically by cancer/tumor cells, but not by normal cells. TSA can be specifically recognized by neoantigen-specific T cell receptors (TCRs) in the context of major histocompatibility complexes (MHCs) molecules. Accordingly, TSA include in particular neoantigens. In general neoantigens are antigens, which were not present before and are, thus, “new” to the immune system. Neoantigens are typically due to somatic mutations. In the context of cancer/tumors, cancer/tumor-specific neoantigens were typically not present before the cancer/tumor developed and cancer/tumor-specific neoantigens are usually encoded by somatic gene mutations in the cancerous cells/tumor cells. From an immunological perspective, a tumor neoantigen is the truly foreign protein and entirely absent from normal human organs/tissues. For most human tumors without a viral etiology, tumor neoantigens can e.g. derive from a variety of nonsynonymous genetic alterations including single-nucleotide variants (SNVs), insertions and deletions (indel), gene fusions, frameshift mutations, and structural variants (SVs). For example, tumor-neoantigens may be identified using in silico prediction tools known in the art as disclosed in Trends in Molecular Medicine, November 2019, Pages 980-992 or by methods known to the skilled person, such as cancer genome sequencing or deep-sequencing technologies identifying mutations within the protein-coding part of the (cancer) genome.

Suitable cancer/tumor epitopes can also be retrieved for example from cancer/tumor epitope databases, e.g. from the Cancer Antigenic Peptide Database (Vigneron N, Stroobant V, Van den Eynde B J, van der Bruggen P. Database of T cell-defined human tumor antigens: the 2013 update. Cancer Immun. 2013; 13:15), or from the database “Tantigen” (Zhang G, Chitkushev L, Olsen L R, Keskin D B, Brusic V. TANTIGEN 2.0: a knowledge base of tumor T cell antigens and epitopes. BMC Bioinformatics. 2021; 22(Suppl 8):40. Published 2021 Apr. 14. doi:10.1186/s12859-021-03962-7).

In some embodiments, the cancer/tumor antigen or the cancer/tumor epitope may be a recombinant cancer/tumor antigen or a recombinant cancer/tumor epitope. Such a recombinant cancer/tumor antigen or a recombinant cancer/tumor epitope may be designed by introducing mutations that change (add, delete or substitute) particular amino acids in the overall amino acid sequence of the native cancer/tumor antigen or the native cancer/tumor epitope. The introduction of mutations does not alter the cancer/tumor antigen or the cancer/tumor epitope so much that it cannot be universally applied across a mammalian subject, and preferably a human or dog subject, but changes it enough that the resulting amino acid sequence breaks tolerance or is considered a foreign antigen in order to generate an immune response. Another manner may be creating a consensus recombinant cancer/tumor antigen or cancer/tumor epitope that has at least 85% and up to 99% amino acid sequence identity to its' corresponding native cancer/tumor antigen or native cancer/tumor epitope; preferably at least 90% and up to 98% sequence identity; more preferably at least 93% and up to 98% sequence identity; or even more preferably at least 95% and up to 98% sequence identity. In some instances the recombinant cancer/tumor antigen or the recombinant cancer/tumor epitope has 95%, 96%, 97%, 98%, or 99% amino acid sequence identity to its' corresponding native cancer/tumor antigen or cancer/tumor epitope. The native cancer/tumor antigen is the antigen normally associated with the particular cancer or tumor. Depending upon the cancer/tumor antigen, the consensus sequence of the cancer/tumor antigen can be across mammalian species or within subtypes of a species or across viral strains or serotypes. Some cancer/tumor antigen do not vary greatly from the wild type amino acid sequence of the cancer/tumor antigen. The aforementioned approaches can be combined so that the final recombinant cancer/tumor antigen or cancer/tumor epitope has a percent similarity to native cancer antigen amino acid sequence as discussed above. In other embodiments, the amino acid sequence of an epitope of a cancer/tumor antigen as described herein is not mutated and, thus, identical to the reference epitope sequence.

Similarly, as the ATP-hydrolyzing enzyme, also the antigen, or the fragment thereof comprising at least one epitope, may be administered as protein/peptide or encoded in a nucleic acid; or a host cell, a microorganism or a viral particle may be used for delivery (of such a nucleic acid). The detailed description of the nucleic acid, the host cell, microorganism and viral particle above (in the context of the ATP hydrolyzing enzyme) applies accordingly for the antigen or the fragment thereof comprising at least one epitope. While the form of administration for the ATP-hydrolyzing enzyme and the antigen, or the fragment thereof comprising at least one epitope, may be selected independently from each other, both may be administered in corresponding forms, e.g. both as protein/peptide or as nucleic acid etc. In some embodiments, the combination may comprise a host cell or a microorganism comprising a first nucleic acid comprising a polynucleotide encoding the ATP hydrolyzing enzyme and a second nucleic acid comprising a polynucleotide encoding the antigen or the fragment thereof comprising at least one antigenic epitope. Accordingly, the combination may comprise a host cell or a microorganism (heterologously) expressing the ATP hydrolyzing enzyme and the antigen or the fragment thereof comprising at least one antigenic epitope.

In some embodiments, the combination of (i) the immune checkpoint modulator as described herein and (ii) the ATP-hydrolyzing enzyme, a nucleic acid encoding the ATP-hydrolyzing enzyme, or a host cell, microorganism or viral particle comprising the nucleic acid encoding the ATP-hydrolyzing enzyme, as described herein, does not comprise vancomycin (or an antibiotic). In other words, the administration of vancomycin (or an antibiotic) may be avoided when the combination according to the present invention as described herein is administered.

### Kits

In a further aspect, the present invention also provides a kit comprising:
(i) an immune checkpoint inhibitor; and
(ii) any of:
   (a) an ATP hydrolyzing enzyme,
   (b) a nucleic acid comprising a polynucleotide encoding the ATP hydrolyzing enzyme,
   (c) a host cell comprising the nucleic acid,
   (d) a microorganism comprising the nucleic acid, or
   (e) a viral particle comprising the nucleic acid.

In some embodiments, such a kit comprises (i) the immune checkpoint modulator as described above and (ii) an ATP-hydrolyzing enzyme as described above. In some embodiments, such a kit comprises (i) the immune checkpoint modulator as described above and (ii) a nucleic acid as described above encoding the ATP-hydrolyzing enzyme. In some embodiments, such a kit comprises (i) the immune checkpoint modulator as described above and (ii) a host cell as described above comprising a nucleic acid comprising a polynucleotide encoding the ATP hydrolyzing enzyme. In some embodiments, such a kit comprises (i) the immune checkpoint modulator as described above and (ii) a microorganism as described above comprising a nucleic acid comprising a polynucleotide encoding the ATP hydrolyzing enzyme. In some embodiments, such a kit comprises (i) the immune checkpoint modulator as described above and (ii) a viral particle as described above comprising a nucleic acid comprising a polynucleotide encoding the ATP hydrolyzing enzyme. Accordingly, the detailed embodiments of the immune checkpoint modulator as described above apply accordingly to the kit according to the present invention. Accordingly, the detailed embodiments of the ATP-hydrolyzing enzyme as described above, the nucleic acid as described above encoding the ATP-hydrolyzing enzyme, or the host cell as described above, the microorganism as described above or the viral particle as described above apply accordingly to the kit according to the present invention. In particular, (i) the immune checkpoint modulator and/or (ii) the ATP-hydrolyzing enzyme as described above, the nucleic acid as described above encoding the ATP-hydrolyzing enzyme, or the host cell as described above, the microorganism as described above or the viral particle as described above may be provided in a composition (or in separate compositions) as described above.

Moreover, the kit may further comprise any one (or a combination) of:
(a) an antigen or a fragment thereof comprising at least one epitope as described above,
(b) a nucleic acid comprising a polynucleotide encoding the antigen or the fragment thereof comprising at least one epitope as described above,
(c) a host cell comprising the nucleic acid as described above,
(d) a microorganism comprising the nucleic acid as described above, or
(e) a viral particle comprising the nucleic acid as described above.

It is understood that the detailed description of the antigen or the fragment thereof as above, applies accordingly.

The various components of the kit may be packaged in one or more containers. In some embodiments, the different components; in particular components (i) and (ii), i.e. (i) the immune checkpoint modulator as described herein and (ii) the ATP hydrolyzing enzyme, the nucleic acid, the host cell, the microorganism or the viral particle as described herein; are provided in distinct containers. The distinct containers with the components may be provided together, e.g. in a box/container. The above components may be provided in a lyophilized or dry form or dissolved in a suitable buffer. For example, the kit may comprise a (pharmaceutical) composition comprising the immune checkpoint modulator as described above and a (pharmaceutical) composition comprising any of the ATP-hydrolyzing enzyme as described above, the nucleic acid as described above encoding the ATP-hydrolyzing enzyme, or the host cell as described above, the microorganism as described above or the viral particle as described above, e.g. with each composition in a separate container. The kit may also comprise a (pharmaceutical) composition comprising both, the immune checkpoint modulator and any of the ATP-hydrolyzing enzyme as described above, the nucleic acid as described above encoding the ATP-hydrolyzing enzyme, or the host cell as described above, the microorganism as described above or the viral particle as described above.

The kit may also comprise additional reagents including, for instance, buffers for storage and/or reconstitution of the above-referenced components, washing solutions, and the like.

In addition, the kit-of-parts according to the present invention may optionally contain instructions of use. Preferably, the kit further comprises a package insert or label with directions to treat a cancer by using a combination of (i) the immune checkpoint modulator and (ii) the ATP-hydrolyzing enzyme as described above, the nucleic acid as described above encoding the ATP-hydrolyzing enzyme, or the host cell as described above, the microorganism as described above or the viral particle as described above. For example, the directions to use the combination according to the present invention as described above may include an administration regimen.

### Medical Treatment and Uses

The combinations of the invention as described above and the kit of the invention as described above may be used in medicine, for example for the treatment of cancer.

The combination of (i) the immune checkpoint modulator as described above and (ii) the ATP-hydrolyzing enzyme as described above, the nucleic acid as described above encoding the ATP-hydrolyzing enzyme, or the host cell as described above, the microorganism as described above or the viral particle as described above is able to initiate or enhance the efficacy of checkpoint modulators as shown in the examples.

Accordingly, the present invention also provides a method for reducing the risk of occurrence, treating, ameliorating, or reducing cancer or initiating, enhancing or prolonging an anti-tumor-response in a subject in need thereof, comprising administering to the subject
(i) an immune checkpoint modulator; and
(ii) (a) an ATP hydrolyzing enzyme,
   (b) a nucleic acid comprising a polynucleotide encoding the ATP hydrolyzing enzyme,
   (c) a host cell comprising the nucleic acid,
   (d) a microorganism comprising the nucleic acid, or
   (e) a viral particle comprising the nucleic acid.

In addition, the present invention also provides a combination therapy for reducing the risk of occurrence, treating, ameliorating, or reducing cancer or initiating, enhancing or prolonging an anti-tumor-response, wherein the combination therapy comprises administration of
(i) an immune checkpoint modulator; and
(ii) (a) an ATP hydrolyzing enzyme,
   (b) a nucleic acid comprising a polynucleotide encoding the ATP hydrolyzing enzyme,
   (c) a host cell comprising the nucleic acid,
   (d) a microorganism comprising the nucleic acid, or
   (e) a viral particle comprising the nucleic acid.

Accordingly, the present invention also provides an immune checkpoint modulator for use in medicine, wherein the immune checkpoint modulator is administered in combination with
(a) an ATP hydrolyzing enzyme,
(b) a nucleic acid comprising a polynucleotide encoding the ATP hydrolyzing enzyme,
(c) a host cell comprising the nucleic acid,
(d) a microorganism comprising the nucleic acid, or
(e) a viral particle comprising the nucleic acid.

Preferably, the immune checkpoint modulator used in combination as described above, is used for the treatment of a cancer.

It is understood that the (detailed) description above of (i) the immune checkpoint modulator and (ii) the ATP hydrolyzing enzyme, the nucleic acid, the host cell, the microorganism or the viral particle (in the context of the “combination”) as well as regarding respective composition and administration forms applies accordingly to the immune checkpoint modulator for use in combination as described herein. Likewise, it may be further combined with the antigen or the fragment thereof (administered in any one of the forms described above), as described above. Also further details as described above apply accordingly.

The treatment of cancer may be prophylactic treatment (e.g., reducing the risk of occurrence of a cancer) or therapeutic treatment. As used herein, the term “therapeutic treatment” refers to treatment after the onset of a disease, while “prophylactic treatment” refers to treatment before the onset of a disease or before the first symptoms occur. In particular, “therapeutic treatment” does not include prophylactic measures applied before the onset of a disease. Since the onset of a disease is often associated with symptom(s) of the disease, human or animal subjects are often “therapeutically” treated after the diagnosis or at least a (strong) assumption that the subject suffers from a certain disease. Therapeutic treatment aims in particular at (1) ameliorating, improving, or curing a disease (state) or (2) at inhibiting or delaying the progression of a disease (for example, by increasing the average survival time for cancer patients). However, prevention of the onset of a disease cannot typically be achieved by therapeutic treatment.

The combination as described may be used (for the preparation of a medicament) for the treatment of a cancer disease. The term “disease” as used in the context of the present invention is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

Cancer diseases (or “cancers”) are a group of diseases involving abnormal cell growth, in particular with the potential to invade or spread to other parts of the body. Cancerous cells/tissue may typically show the six hallmarks of cancer, namely (a) cell growth and division absent the proper signal; (b) continuous growth and division even given contrary signals; (c) avoidance of programmed cell death; (d) limitless number of cell divisions; (e) promoting blood vessel construction; and (f) invasion of tissue and formation of metastases.

Cancer diseases include diseases caused by defective apoptosis. The cancer may be a solid tumor, a blood cancer, or a lymphatic cancer. In the context of the present invention, the cancer to be treated may preferably be a solid tumor. The cancer to be treated may be metastatic.

Preferably, in the treatment of a cancer, the combination according to the present invention inhibits, reduces or delays the ongoing/further growth of a tumor (or of metastases). The combination of the invention may also decrease the size of the tumor (or the number of metastases). In some embodiments, the combination of the invention may reduce the risk of or prevent the reoccurrence of the tumor and/or metastases.

Non-limiting examples of cancer diseases include melanoma; intestinal cancer, including tumors of the small intestine and gastrointestinal tumors, such as colon carcinoma, colorectal cancer, colon adenocarcinoma; anal carcinoma; brain tumors, such as glioblastomas, breast cancer; adenocarcinoma (e.g., colon adenocarcinoma); genital tumors, including cancers of the genitourinary tract, such as prostate cancer; liver cancer and lung cancer.

As described above, a “combination” of (i) the immune checkpoint modulator as described above and (ii) the ATP-hydrolyzing enzyme as described above, the nucleic acid as described above encoding the ATP-hydrolyzing enzyme, or the host cell as described above, the microorganism as described above or the viral particle as described above means that the treatment with (i) the immune checkpoint modulator as described herein is combined with the treatment with (ii) the ATP-hydrolyzing enzyme as described above, the nucleic acid as described above encoding the ATP-hydrolyzing enzyme, or the host cell as described above, the microorganism as described above or the viral particle as described above. In other words, even if one of the components (i) or (ii) ((i) the checkpoint modulator and (ii) the ATP-hydrolyzing enzyme as described above, the nucleic acid as described above encoding the ATP-hydrolyzing enzyme, or the host cell as described above, the microorganism as described above or the viral particle as described above) is not administered, e.g., at the same day as the other component (the other of (i) or (ii)), their treatment schedules are intertwined. This means that “a combination” in the context of the present invention does in particular not include the start of a treatment with one component (i) or (ii), when the treatment with the other component of the components (i) and (ii) is already finished. In more general, an “intertwined” treatment schedule of the components (i) and (ii)—and, thus, a combination of the components (i) and (ii)—means that:
(i) the first administration of the ATP-hydrolyzing enzyme, the nucleic acid encoding the ATP-hydrolyzing enzyme, or the host cell, microorganism or viral particle comprising the nucleic acid encoding the ATP-hydrolyzing enzyme starts not more than one week (preferably not more than 3 days, more preferably not more than 2 days, even more preferably not more than a day) after the (final) treatment with the immune checkpoint modulator (e.g., the final administration of the immune checkpoint modulator); or
(ii) the first administration of the immune checkpoint modulator starts not more than one week (preferably not more than 3 days, more preferably not more than 2 days, even more preferably not more than a day) after the (final) treatment with the ATP-hydrolyzing enzyme, the nucleic acid encoding the ATP-hydrolyzing enzyme, or the host cell, microorganism or viral particle comprising the nucleic acid encoding the ATP-hydrolyzing enzyme (e.g., the final administration of the ATP-hydrolyzing enzyme, the nucleic acid encoding the ATP-hydrolyzing enzyme, or the host cell, microorganism or viral particle comprising the nucleic acid encoding the ATP-hydrolyzing enzyme).

For example, in the combination of (i) the immune checkpoint modulator as described herein and of (ii) the ATP-hydrolyzing enzyme, the nucleic acid encoding the ATP-hydrolyzing enzyme, or the host cell, microorganism or viral particle comprising the nucleic acid encoding the ATP-hydrolyzing enzyme, one component ((i) or (ii)) may be administered once or twice a week (e.g., (i) the immune checkpoint modulator), while the other component may be administered daily (e.g., (ii) the ATP-hydrolyzing enzyme, the nucleic acid encoding the ATP-hydrolyzing enzyme, or the host cell, microorganism or viral particle comprising the nucleic acid encoding the ATP-hydrolyzing enzyme). In this example, on some days of the daily administration of one component also the other component is administered. However, in another example, if both components were administered weekly, in some of the weeks both components were administered (even if not administered at the same day, the treatment schedules still overlap). If one of the components is administered only once, while the other component is administered repeatedly, the single administration of one component usually lies within the treatment cycle of the other component (even if not administered at the same day). In general, to achieve a combination, one component may be administered as long as its effects overlap with the effects of the other component.

As outlined above, the administration of (i) the immune checkpoint modulator as described herein and/or of (ii) the ATP-hydrolyzing enzyme, the nucleic acid encoding the ATP-hydrolyzing enzyme, or the host cell, microorganism or viral particle comprising the nucleic acid encoding the ATP-hydrolyzing enzyme may require repeated (multiple, i.e. more than one) administrations, e.g. multiple injections and/or multiple oral administrations. Thus, the administration may be repeated at least two times, for example once as primary immunization injections and, later, as booster injections; or, e.g., in a daily manner. Accordingly, (i) the immune checkpoint modulator as described herein and (ii) the ATP-hydrolyzing enzyme, the nucleic acid encoding the ATP-hydrolyzing enzyme, or the host cell, microorganism or viral particle comprising the nucleic acid encoding the ATP-hydrolyzing enzyme may be administered repeatedly or continuously. The immune checkpoint modulator as described herein and the ATP-hydrolyzing enzyme, the nucleic acid encoding the ATP-hydrolyzing enzyme, or the host cell, microorganism or viral particle comprising the nucleic acid encoding the ATP-hydrolyzing enzyme may be administered repeatedly or continuously for a period of at least 1, 2, 3, or 4 weeks; 2, 3, 4, 5, 6, 8, 10, or 12 months; or 2, 3, 4, or 5 years. For example, the immune checkpoint modulator may be administered twice per day, once per day, every two days, every three days, once per week, every two weeks, every three weeks, once per month or every two months. For example, the ATP-hydrolyzing enzyme, the nucleic acid encoding the ATP-hydrolyzing enzyme, or the host cell, microorganism or viral particle comprising the nucleic acid encoding the ATP-hydrolyzing enzyme may be administered twice per day, once per day (e.g., daily), every two days, every three days, once per week, every two weeks, every three weeks, once per month or every two months.

In some embodiments, (i) the immune checkpoint modulator; and/or (ii) the ATP hydrolyzing enzyme, the nucleic acid, the host cell, the microorganism or the viral particle are administered on the same day. In some embodiments, (i) the immune checkpoint modulator; and/or (ii) the ATP hydrolyzing enzyme, the nucleic acid, the host cell, the microorganism or the viral particle are administered repeatedly. For example, the ATP hydrolyzing enzyme, the nucleic acid, the host cell, the microorganism or the viral particle may be administered daily, while the immune checkpoint modulator may be administered once or twice a week on days, on which also the other component (the ATP hydrolyzing enzyme, the nucleic acid, the host cell, the microorganism or the viral particle) is administered.

In some embodiments, (i) the immune checkpoint modulator; and (ii) the ATP hydrolyzing enzyme, the nucleic acid, the host cell, the microorganism or the viral particle may be administered at about the same time. “At about the same time”, as used herein, means in particular simultaneous administration or that directly after administration of component (i) component (ii) is administered or vice versa. The skilled person understands that “directly after” includes the time necessary to prepare the second administration—for example the time necessary for exposing and disinfecting the location for the second administration as well as appropriate preparation of the “administration device” (e.g., syringe, pump, etc.). Simultaneous administration also includes if the periods of administration of both components overlap or if, for example, one component is administered over a longer period of time, such as 30 min, 1 h, 2 h or even more, e.g. by infusion, and the other component is administered at some time during such a long period.

Preferably, (i) the immune checkpoint modulator; and (ii) the ATP hydrolyzing enzyme, the nucleic acid, the host cell, the microorganism or the viral particle are administered consecutively. For example, (i) the immune checkpoint modulator may be administered before (ii) the ATP hydrolyzing enzyme, the nucleic acid, the host cell, the microorganism or the viral particle is administered; or (i) the immune checkpoint modulator may be administered after (ii) the ATP hydrolyzing enzyme, the nucleic acid, the host cell, the microorganism or the viral particle is administered. In consecutive administration, the time interval between administration of both components (i) and (ii) is preferably no more than one week, more preferably no more than 3 days, even more preferably no more than 2 days and most preferably no more than 24 h are in between administration of both components (i) and (ii). It is particularly preferred that (i) the checkpoint modulator and (ii) the ATP hydrolyzing enzyme, the nucleic acid, the host cell, the microorganism or the viral particle are administered at the same day. The time between administration of both components (i) and (ii) may be no more than 12 hours, preferably no more than 6 hours, more preferably no more than 3 hours, e.g. no more than 2 hours or no more than 1 hour.

The immune checkpoint modulator; and the ATP hydrolyzing enzyme, the nucleic acid, the host cell, the microorganism or the viral particle can be administered by various routes of administration, for example, systemically or locally. Routes for systemic administration in general include, for example, enteral and parenteral routes, which include subcutaneous, intravenous, intramuscular, intraarterial, intradermal and intraperitoneal routes. Routes for local administration in general include, for example, administration directly at the site of affliction, such as intratumoral administration.

Preferably, (i) the immune checkpoint modulator; and (ii) the ATP hydrolyzing enzyme, the nucleic acid, the host cell, the microorganism or the viral particle are administered via distinct routes of administration.

In particular, the ATP hydrolyzing enzyme, the nucleic acid, the host cell, the microorganism or the viral particle is preferably administered via an enteral route of administration. Enteral routes of administration refers to administration via the gastrointestinal tract and includes, for example oral, sublingual, and rectal administration as well as administration via a gastric tube. Oral administration of the ATP hydrolyzing enzyme, the nucleic acid comprising the polynucleotide encoding the ATP hydrolyzing enzyme, the host cell comprising the nucleic acid comprising the polynucleotide encoding the ATP hydrolyzing enzyme, the microorganism comprising the nucleic acid comprising the polynucleotide encoding the ATP hydrolyzing enzyme, or the viral particle comprising the nucleic acid comprising the polynucleotide encoding the ATP hydrolyzing enzyme is preferred. Without being bound to any theory, it is assumed that the ATP-hydrolyzing enzyme mediates its beneficial effects (when combined with a checkpoint inhibitor) in the intestinal lumen, namely, by degrading extracellular ATP released from microbiota in the gut. The experimental data of this specification demonstrate the crucial role of the ATP-hydrolyzing enzyme on the ATP released from microbiota in the gut in order to mediate its beneficial effects on the activity of the checkpoint inhibitor. Because enteral administration of the ATP hydrolyzing enzyme, the nucleic acid, the host cell, the microorganism or the viral particle delivers the ATP hydrolyzing enzyme into the gastrointestinal tract (gut), this route of administration is preferred for the ATP hydrolyzing enzyme, the nucleic acid, the host cell, the microorganism or the viral particle.

For example, the (encoded) ATP hydrolyzing enzyme may be a soluble ATP hydrolyzing enzyme; and the ATP hydrolyzing enzyme, the nucleic acid, the host cell, the microorganism or the viral particle may be administered via an enteral route of administration.

The immune checkpoint modulator is preferably administered via a parenteral route of administration. Non-limiting examples of parental administration include intravenous, intraarterial, intramuscular, intradermal, intranodal, intraperitoneal, and subcutaneous routes of administration. Preferably, the immune checkpoint modulator may be administered intravenously or subcutaneously. The checkpoint modulator may also be administered at the site of affliction, e.g. intratumorally.

In certain embodiments, (i) the immune checkpoint modulator; and (ii) the ATP hydrolyzing enzyme, the nucleic acid, the host cell, the microorganism or the viral particle are administered via the same route of administration, such as any one of the enteral or parental route described above.

The immune checkpoint modulator; and the ATP hydrolyzing enzyme, the nucleic acid, the host cell, the microorganism or the viral particle may be provided in the same or in distinct compositions. Preferably, (i) the immune checkpoint modulator; and (ii) the ATP hydrolyzing enzyme, the nucleic acid, the host cell, the microorganism or the viral particle as described above are provided in distinct compositions, e.g. as described above. Thereby, different other components, e.g. different vehicles, can be used for (i) the immune checkpoint modulator; and (ii) the ATP hydrolyzing enzyme, the nucleic acid, the host cell, the microorganism or the viral particle as described above. Moreover, (i) the immune checkpoint modulator; and (ii) the ATP hydrolyzing enzyme, the nucleic acid, the host cell, the microorganism or the viral particle as described above can be administered via different routes of administration and the doses (in particular the relation of the doses) can be adjusted according to the actual need.

The inventive combination of (i) the immune checkpoint modulator; and (ii) the ATP hydrolyzing enzyme, the nucleic acid, the host cell, the microorganism or the viral particle as described above may be administered as “stand-alone” combination therapy (i.e., without the combination of any further components or active agents, such as anti-cancer agents (e.g., cytostatic agents) or antibodies against tumor-associated antigens). Alternatively, the inventive combination of (i) the immune checkpoint modulator; and (ii) the ATP hydrolyzing enzyme, the nucleic acid, the host cell, the microorganism or the viral particle as described above may be administered in combination with one or more further active agents, such as anti-cancer agents (e.g., cytostatic agents) or antibodies against tumor-associated antigens).

In certain embodiments, the inventive combination of (i) the immune checkpoint modulator; and (ii) the ATP hydrolyzing enzyme, the nucleic acid, the host cell, the microorganism or the viral particle as described above may be combined with adoptive cell therapy, preferably with CAR T cell therapy or with the infusion of in vitro expanded tumor infiltrating T cells. Adoptive cell therapy (also known as “cellular immunotherapy”) makes use of human immune cells for the treatment of cancer. The immune cells are preferably autologous (i.e. they are isolated from the same patient, who receives them after in vitro treatment of the cells), but may also be allogenic (i.e. they are isolated from the another human patient). After isolation, the immune cells may be in vitro expanded and/or genetically engineered (e.g., to enhance their anti-tumor effects). Examples of adoptive cell therapy include tumor-infiltrating lymphocyte (TIL) therapy, engineered T cell receptor (TCR) therapy, chimeric antigen receptor (CAR) T cell therapy and natural killer (NK) cell therapy.

Preferably, the inventive combination of (i) the immune checkpoint modulator; and (ii) the ATP hydrolyzing enzyme, the nucleic acid, the host cell, the microorganism or the viral particle as described above may be combined with adoptive T cell therapy, such as tumor-infiltrating lymphocyte (TIL) therapy, engineered T cell receptor (TCR) therapy, or chimeric antigen receptor (CAR) T cell therapy.

In tumor-infiltrating lymphocyte (TIL) therapy, naturally occurring T cells that have already infiltrated the patient's tumor are harvested. After activating and expanding the tumor-infiltrating T cells, large numbers of these activated T cells can be re-infused to the patient.

In engineered TCR therapy, the T cells are in vitro engineered to introduce an engineered T cell receptor (TCR) to enable the cells to target specific cancer antigens. Thereby, an optimal target for each patient's tumor may be selected and distinct types of T cells may be engineered, such that the treatment can be further improved and personalized to individuals.

In chimeric antigen receptor (CAR) T cell therapy, a key advantage of CARs is their ability to bind to cancer cells even if their antigens are not presented on the surface via MHC, which can render more cancer cells vulnerable to their attacks. CAR T cell therapy targets adoptively transferred T cells directly to tumor cells to provide effective and durable anti-tumor responses. The CAR endows transferred cells with high-avidity binding to cell-surface antigens independently from expression of major histocompatibility complex (MHC) and triggers robust T cell activation and anti-tumor response.

In adoptive cell therapy, the administration of the inventive combination of (i) the immune checkpoint modulator; and (ii) the ATP hydrolyzing enzyme, the nucleic acid, the host cell, the microorganism or the viral particle as described above may be started at the same day when the (T) cells are administered to the patient (after in vitro treatment of the (T) cells). This means that at least one of the two components (i) and (ii) of the inventive combination (e.g. the ATP hydrolyzing enzyme, the nucleic acid, the host cell, the microorganism or the viral particle as described above) may be administered for the first time at the same day when the patient receives the (T) cells after their in vitro treatment. Alternatively, at least one of the two components (i) and (ii) of the inventive combination (or both) may be administered for the first time before the patient receives the (T) cells after their in vitro treatment. In this case, the treatment with the inventive combination may continue while the patient receives the (T) cells after their in vitro treatment. It is also possible to administer at least one of the two components (i) and (ii) of the inventive combination (or both) for the first time after the patient receives the (T) cells after their in vitro treatment. In particular if both components (i) and (ii) of the inventive combination are administered for the first time after the patient receives the (T) cells after their in vitro treatment, it is preferred that no more than two weeks, preferably no more than one week, more preferably, no more than 5 days, even more preferably no more than 2 or 3 days are between the administration of the (T) cells after their in vitro treatment and the administration of the first of the two components (i) and (ii) of the inventive combination.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1 shows a map of the pHND10 plasmid carrying the phoN2 gene encoding periplasmic ATP-diphosphohydrolase (apyrase).

FIG. 2 shows the amino acid sequence of wild-type phon2 protein (apyrase; SEQ ID NO: 1) and indicates the position of the R192P substitution in the loss-of-function isoform (SEQ ID NO: 2).

FIG. 3 shows the nucleotide sequence of the phoN2 gene (SEQ ID NO: 3) used for generating pHND10 plasmid.

FIG. 4 shows for Example 2 the development of tumor sizes over time. Mice were inoculated subcutaneously (s.c.) with 1×10⁶ B16-OVA melanoma cells and on day 8, 11, 14 and 18 after tumor inoculation treated with PBS (B16-OVA) or 100 μg of anti-PD-L1 antibody in 100 μl PBS intraperitoneally (i.p.). Mice were also gavaged everyday with 1×10¹⁰ of E. coli^{pApyr} or E. coli^{pHND19} (as indicated) or PBS from day 5 until the end of the experiment. Tumor growth was monitored until the experimental endpoint. Two-way ANOVA for the statistical analysis of tumor growth was applied. n=15 (B16-OVA); 19 (B16-OVA+aPDL1); 20 (B16-OVA+aPDL1+E. coli^{pHND19} or E. coli^{pAPYR}). ***p<0.001 , ****p<0.0001.

FIG. 5 shows the survival rates for Example 2. Mice were inoculated s.c. with 1×10⁶ B16-OVA melanoma cells and on day 8, 11, 14 and 18 after tumor inoculation treated with PBS (B16-OVA) or 100 μg of anti-PD-L1 antibody in 100 μl PBS i.p. Mice were also gavaged everyday with 1×10¹⁰ of E. coli^{pApyr} or E. coli^{pHND19} (as indicated) or PBS from day 5 until the end of the experiment and survival monitored. Mantel-Cox log-rank test for the statistical analysis of survival curves was applied. n=18 (B16-OVA); 20 (B16-OVA+aPDL1 and B16-OVA+aPDL1+E. coli^{pHND19} or E. coli^{pAPYR}). *p<0.05, **p<0.01.

FIG. 6 shows for Example 3 the development of tumor sizes over time. Mice were inoculated s.c. with 1×10⁶ MC38 colon adenocarcinoma cells and on day 8, 11, 14 and 18 after tumor inoculation treated with PBS (MC38) or 100 μg of anti-PD-L1 antibody in 100 μl PBS i.p. Mice were also gavaged everyday with 1×10¹⁰ of E. coli^{pApyr} or E. coli^{pHND19} (as indicated) or PBS from day 5 until the end of the experiment. Tumor growth was monitored until the experimental endpoint. Two-way ANOVA for the statistical analysis of tumor growth was applied. n=15 (MC38); 22 (MC38+aPDL1); 23 (MC38+aPDL1+E. coli^{pHND19}); 24 (MC38+aPDL1+E. coli^{pAPYR}). **p<0.1 , ***p<0.001 FIG. 7 shows the survival rates for Example 3. Mice were inoculated s.c. with 1×10⁶ MC38 colon adenocarcinoma cells and on day 8, 11, 14 and 18 after tumor inoculation treated with PBS (MC38) or 100 μg of anti-PD-L1 antibody in 100 μl PBS i.p. Mice were also gavaged everyday with 1×10¹⁰ of E. coli^{pApyr} or E. coli^{pHND19} (as indicated) or PBS from day 5 until and survival monitored. Mantel-Cox log-rank test for the statistical analysis of survival curves was applied. n=9 (MC38); 16 (MC38+aPDL1 and MC38+aPDL1⁺ E. coli^{pHND19}); 15 (MC38+aPDL1+E. coli^{pAPYR}). *p<0.05.

FIG. 8 shows for Example 4 the development of tumor sizes over time. Mice were inoculated s.c. with 1×10⁶ MC38 colon adenocarcinoma cells and on day 8, 11 and 14 after tumor inoculation treated with PBS (MC38) or 100 μg of anti-PD-L1 antibody in 100 μl PBS i.p. Mice were also gavaged everyday with 1×10¹⁰ of E. coli Nissle 1917 or E. coli Nissle 1917^{pApyr} (as indicated) or PBS from day 5 until the end of the experiment. Tumor growth was monitored until the experimental endpoint. Two-way ANOVA for the statistical analysis of tumor growth was applied. n=9 (MC38); 11 (MC38+aPDL1 and MC38+aPDL1+E. coli Nissle 1917); 12 (MC38+aPDL1+E. coli Nissle 1917^{pApyr}). *p<0.05.

FIG. 9 shows for Example 5 the development of tumor sizes over time. Mice were inoculated s.c. with 1×10⁶ MC38 colon adenocarcinoma cells and on day 8, 11 and 14 after tumor inoculation treated with PBS (MC38) or 100 μg of anti-PD-L1 antibody in 100 μl PBS i.p. Mice were also gavaged everyday with 1×10¹⁰ of E. coli^{pApyr} or 100 μl periplasmic extract (APY extract) or PBS from day 5 until the end of the experiment. Tumor growth was monitored until the experimental endpoint. Two-way ANOVA for the statistical analysis of tumor growth was applied. n=5 (MC38 and MC38+aPDL1); 6 (MC38+aPDL1+APY extract or E. coli^{pAPYR}). **p<0.01.

FIG. 10 shows for Example 6 representative flow cytometry histograms of electronically gated TCRβ⁺CD8⁺ TILs for CXCR5 expression in mice bearing MC38 tumors and treated with anti-PD-L1, anti-PD-L1 and E. coli^{pHND19} (E. coli p19) or E. coli^{pApyr}. Numbers indicate the percentage of positive cells beyond the displayed marker.

FIG. 11 shows for Example 6 the statistical analysis of the frequency of CXCR5⁺ cells among TCRβ⁺CD8⁺ TILs and CXCR5 expression levels measured as mean fluorescence intensity (MFI) in flow cytometry, in mice bearing MC38 tumors and treated with anti-PD-L1, anti-PD-L1 and E. coli^{pHND19} (E. coli p19) or E. coli^{pApyr}. Two-tailed Mann-Whitney U test. **** p<0.0001. Accordingly, administration of E. coli^{pApyr} results in increase of CXCR5⁺ cells and CXCR5 expression levels among CD8⁺ TILs.

FIG. 12 shows for Example 6 representative flow cytometry histograms of TCF1 expression in CXCR5⁻ (empty curve) and CXCR5⁺ (grey curve) subsets of electronically gated CD8⁺ TILs in MC38 tumors from mice treated with anti-PD-L1 and E. coli^{pApyr}. On the right, statistical analysis of MFI in CXCR5⁻ and CXCR5⁺ cells within CD8⁺ TILs from mice bearing MC38 tumors and treated with anti-PD-L1, anti-PD-L1 and E. coli^{pHND19} or E. coli^{pApyr}. Two-tailed Mann-Whitney U test. **** p<0.0001.

FIG. 13 shows for Example 7 representative flow cytometry histograms of electronically gated TCRβ⁺CD8⁺ cells of Peyer's patches of the ileum for CXCR5 expression in mice treated with anti-PD-L1, anti-PD-L1 and E. coli^{pHND19} (E. coli p19), anti-PD-L1 and E. coli^{pApyr}. Numbers indicate the percentage of positive cells beyond the displayed marker.

FIG. 14 shows for Example 7 the statistical analysis of the frequency of CXCR5⁺ cells in ileal PPs among TCRβ⁺CD8⁺ cells of Peyer's patches of the ileum and CXCR5 expression levels measured as MFI in flow cytometry, in mice treated with anti-PD-L1, anti-PD-L1 and E. coli^{pHND19} (E. coli p19), anti-PD-L1 and E. coli^{pApyr}. Two-tailed Mann-Whitney U test. * p<0.05, ** p<0.01, *** p<0.001.

FIG. 15 shows for Example 8 representative flow cytometry histograms of electronically gated TCRβ⁺CD8⁺ TILs for ICOS expression in mice bearing MC38 tumors and treated with anti-PD-L1, anti-PD-L1 and E. coli^{pHND19} (E. coli p19), anti-PD-L1 and E. coli^{pApyr}. Numbers indicate the percentage of positive cells beyond the displayed marker.

FIG. 16 shows for Example 8 the statistical analysis of the frequency of ICOS⁺ cells among TCRβ⁺CD8⁺ TILs detected in flow cytometry, in mice bearing MC38 tumors and treated with anti-PD-L1, anti-PD-L1 and E. coli^{pHND19} (E. coli p19), anti-PD-L1 and E. coli^{pApyr}. Two-tailed Mann-Whitney U test. *** p<0.001. Accordingly, administration of E. coli^{pApyr} results in increase of ICOS⁺ cells among CD8⁺ TILs.

FIG. 17 shows for Example 9 representative flow cytometry histograms of electronically gated TCRβ⁺CD8⁺ TILs for IFN-γ secretion in mice bearing MC38 tumors and treated with anti-PD-L1, anti-PD-L1 and E. coli^{pHND19} (E. coli p19), anti-PD-L1 and E. coli^{pApyr}. Numbers indicate the percentage of IFN-γ secreting cells.

FIG. 18 shows for Example 9 the statistical analysis of the frequency of IFN-γ secreting cells among TCRβ⁺CD8⁺ TILs detected in flow cytometry, in mice bearing MC38 tumors and treated with anti-PD-L1, anti-PD-L1 and E. coli^{pHND19} (E. coli p19), anti-PD-L1 and E. coli^{pApyr}. Two-tailed Mann-Whitney U test. * p<0.05, ** p<0.01. Accordingly, administration of E. coli^{pApyr} results in increase of IFN-γ secreting cells among CD8⁺ TILs.

FIG. 19 shows for Example 9 representative flow cytometry histograms of electronically gated TCRβ⁺CD8⁺ TILs for IL-21 secretion in mice bearing MC38 tumors and treated with anti-PD-L1, anti-PD-L1 and E. coli^{pHND19} (E. coli p19), anti-PD-L1 and E. coli^{pApyr}. Numbers indicate the percentage of IL-21 secreting cells.

FIG. 20 shows for Example 9 the statistical analysis of the frequency of IL-21 secreting cells among TCRβ⁺CD8⁺ TILs detected by flow cytometry, in mice bearing MC38 tumors and treated with anti-PD-L1, anti-PD-L1 and E. coli^{pHND19} (E. coli p19), anti-PD-L1 and E. coli^{pApyr}. Two-tailed Mann-Whitney U test. * p<0.05. Accordingly, administration of E. coli^{pApyr} results in increase of IL-21 secreting cells among CD8⁺ TILs.

FIG. 21 shows for Example 10 representative flow cytometry histograms of electronically gated TCRβ⁺CD8⁺ cells from PPs for IL-21 secretion in mice treated with anti-PD-L1, anti-PD-L1 and E. coli^{pHND19} (E. coli p19), anti-PD-L1 and E. coli^{pApyr}. Numbers indicate the percentage of IL-21 secreting cells.

FIG. 22 shows for Example 10 the statistical analysis of the frequency of IL-21 secreting cells among TCRβ⁺CD8⁺ cells from ileal PPs of mice treated with anti-PD-L1, anti-PD-L1 and E. coli^{pHND19} (E. coli p19), anti-PD-L1 and E. coli^{pApyr}. Two-tailed Mann-Whitney U test. ** p<0.01. Accordingly, administration of E. coli^{pApyr} results in increase of IL-21 secreting cells among CD8⁺ cells isolated from ileal Peyer's patches.

FIG. 23 shows for Example 11 representative flow cytometry plots of electronically gated CD3⁻ cells for CD11c⁺MHCII⁺ cells in mice bearing MC38 tumors and treated with anti-PD-L1, anti-PD-L1 and E. coli^{pHND19} (E. coli p19), anti-PD-L1 and E. coli^{pApyr}. Numbers indicate the percentage of positive cells in the displayed quadrant.

FIG. 24 shows for Example 11 the statistical analysis of the frequency of CD11c⁺MHCII⁺ cells among CD3⁻ cells detected by flow cytometry, in mice bearing MC38 tumors and treated with anti-PD-L1, anti-PD-L1 and E. coli^{pHND19} (E. coli p19), anti-PD-L1 and E. coli^{pApyr}. Two-tailed Mann-Whitney U test. **p<0.01, ***p<0.001. Accordingly, administration of E. coli^{pApyr} results in increase of CD11c⁺MHCII⁺ cells among CD3-tumor infiltrating leukocytes.

FIG. 25 shows for Example 11 representative flow cytometry plots of electronically gated CD11c⁺MHCII⁺ cells for CD103⁺CD70⁺ cells in mice bearing MC38 tumors and treated with anti-PD-L1, anti-PD-L1 and E. coli^{pHND19} (E. coli p19), anti-PD-L1 and E. coli^{pApyr}. Numbers indicate the percentage of positive cells in the displayed quadrant.

FIG. 26 shows for Example 11 the statistical analysis of the frequency of CD103⁺CD70⁺ cells among CD11c⁺MHCII⁺ cells detected by flow cytometry, in mice bearing MC38 tumors and treated with anti-PD-L1, anti-PD-L1 and E. coli^{pHND19} (E. coli p19), anti-PD-L1 and E. coli^{pApyr}. Two-tailed Mann-Whitney U test. **p<0.01. Accordingly, administration of E. coli^{pApyr} results in increase of CD103⁺CD70⁺ cells among CD11c⁺MHCII⁺ tumor infiltrating cells.

FIG. 27 shows for Example 12 that E. coli^{pApyr} improves therapeutic outcome of adoptive transfer of tumor-specific CD8 cells combined with anti-PD-L1 treatment in mice bearing MC38 colon adenocarcinoma. At day 0, mice were engrafted s.c. with 1×10⁶ OVA expressing MC38 colon adenocarcinoma cells. On day 8, mice were injected i.v. with 8×10⁵ TCR transgenic anti-OVA CD8⁺ OT-I T cells. On day 10, 14, 17 and 20, mice were treated with 100 μg of anti-PD-L1 antibody in 100 μl PBS i.p. Mice were also gavaged everyday with 1×10¹⁰ of E. coli^{pApyr} or PBS from day 8 until the end of the experiment. Tumor growth was monitored until the experimental endpoint. Two-way ANOVA for the statistical analysis of tumor growth was applied. n=7. ***p<0.001.

FIG. 28 shows for Example 13 that E. coli^{pApyr} improves anti-CTLA4 treatment outcome in mice bearing MC38 colon adenocarcinoma. Mice were inoculated s.c. with 1×10⁶ MC38 colon adenocarcinoma cells and on day 8, 11, 14 and 18 after tumor inoculation treated with PBS (MC38) or 100 μg of anti-CTLA4 antibody in 100 μl PBS i.p. Mice were also gavaged everyday with 1×10¹⁰ of E. coli^{pApyr} (as indicated) or PBS from day 5 until the end of the experiment. Tumor growth was monitored until the experimental endpoint. Two-way ANOVA for the statistical analysis of tumor growth was applied. n=5 (MC38); 6 (MC38+aCTLA4); 7 (MC38+aCTLA4+E. coli^{pAPYR}). **p<0.01, ***p<0.001.

FIG. 29 shows for Example 13 that E. coli^{pApyr} improves anti-CTLA4 survival in mice bearing MC38 colon adenocarcinoma. Mice were inoculated s.c. with 1×10⁶ MC38 colon adenocarcinoma cells and on day 8, 11, 14 and 18 after tumor inoculation treated with PBS (MC38) or 100 μg of anti-CTLA4 antibody in 100 μl PBS i.p. Mice were also gavaged everyday with 1×10¹⁰ of E. coli^{pApyr} (as indicated) or PBS from day 5 and survival monitored. Mantel-Cox log-rank test for the statistical analysis of survival curves was applied. n=5 (MC38); 6 (MC38+aCTLA4); 7 (MC38+aCTLA4+E. coli^{pAPYR}). *p<0.05, **p<0.01.

FIG. 30 shows for Example 14 that E. coli^{pApyr} improves outcome of an anti-PD-L1 and anti-CTLA4 combination therapy in mice bearing MC38 colon adenocarcinoma. Mice were inoculated s.c. with 1×10⁶ MC38 colon adenocarcinoma cells and on day 8, 11, 14 and 18 after tumor inoculation treated with PBS (MC38) or 100 μg of anti-PD-L1 and 100 μg of anti-CTLA4 antibody in 100 μl PBS i.p. Mice were also gavaged everyday with 1×10¹⁰ of E. coli^{pApyr} (as indicated) or PBS from day 5 until the end of the experiment. Tumor growth was monitored until the experimental endpoint. Two-way ANOVA for the statistical analysis of tumor growth was applied. n=5 (MC38); 7 (MC38+aPD-L1+aCTLA4); 7 (MC38+aPD-L1+aCTLA4+E. coli^{pAPYR}). *p<0.05, **p<0.01, ***p<0.001.

FIG. 31 shows for Example 14 that E. coli^{pApyr} improves survival by anti-PD-L1 and anti-CTLA4 combination therapy in mice bearing MC38 colon adenocarcinoma. Mice were inoculated s.c. with 1×10⁶ MC38 colon adenocarcinoma cells and on day 8, 11, 14 and 18 after tumor inoculation treated with PBS (MC38) or 100 μg of anti-PD-L1 and 100 μg of anti-CTLA4 antibody in 100 μl PBS i.p. Mice were also gavaged everyday with 1×10¹⁰ of E. coli^{pApyr} (as indicated) or PBS from day 5 and survival monitored. Mantel-Cox log-rank test for the statistical analysis of survival curves was applied. n=5 (MC38); 7 (MC38+aPD-L1+aCTLA4); 7 (MC38+aPD-L1+aCTLA4+E. coli^{pAPYR}). **p<0.01, ***p<0.001.

FIG. 32 shows for Example 15 that E. coli^{pApyr} improves anti-PD-L1 treatment outcome in Balb/c mice bearing CT26 colon adenocarcinoma. Mice were inoculated s.c. with 1×10⁶ CT26 colon adenocarcinoma cells and on day 8, 11, 14 and 17 after tumor inoculation treated with PBS (CT26) or 100 μg of anti-PD-L1 antibody in 100 μl PBS i.p. Mice were also gavaged everyday with 1×10¹⁰ of E. coli^{pApyr} or transformants with empty vector E. coli^{pBAD28} (as indicated) or PBS from day 5 until the end of the experiment. Tumor growth was monitored until the experimental endpoint. Two-way ANOVA for the statistical analysis of tumor growth was applied. n=12 (CT26); 19 (CT26+aPDL1+E. coli^{pBAD28}); 20 (CT26+aPDL1+E. coli^{pAPYR}). *p<0.05, **p<0.01, ***p<0.001.

FIG. 33 shows for Example 15 that E. coli^{pApyr} improves survival by anti-PD-L1 in Balb/c mice bearing CT26 colon adenocarcinoma. Mice were inoculated s.c. with 1×10⁶ CT26 colon adenocarcinoma cells and on day 8, 11, 14 and 17 after tumor inoculation treated with PBS (CT26) or 100 μg of anti-PD-L1 antibody in 100 μl PBS i.p. Mice were also gavaged everyday with 1×10¹⁰ of E. coli^{pApyr} or E. coli^{pBAD28} (as indicated) or PBS from day 5 and survival monitored. Mantel-Cox log-rank test for the statistical analysis of survival curves was applied. n=12 (CT26); 19 (CT26+aPDL1+E. coli^{pBAD28}); 20 (CT26+aPDL1+E. coli^{pAPYR}). *p<0.05, *p<0.001, *p<0.0001.

FIG. 34 shows for Example 16 that administration of E. coli^{pApyr} results in increase of CCR9⁺ cells among CD8⁺ TILs. (A) Representative flow cytometry histograms of electronically gated TCRβ⁺CD8⁺ TILs for CCR9 expression in mice bearing MC38 tumors and treated with anti-PD-L1, anti-PD-L1 and E. coli^{pBAD28} or E. coli^{pApyr}. Numbers indicate the percentage of positive cells beyond the displayed marker. (B) Statistical analysis of the frequency of CCR9⁺ cells among TCRβ⁺CD8⁺ TILs in the indicated mice. Two-tailed Mann-Whitney U test. *** p<0.001.

FIG. 35 shows for Example 17 that administration of E. coli^{pApyr} results in increase of Ki-67⁺ cells among CD8⁺ T cells in the Peyer's patches of the ileum. (A) Representative flow cytometry histograms of electronically gated TCRβ⁺CD8⁺ cells for Ki-67 expression in mice treated with anti-PD-L1, anti-PD-L1 and E. coli^{pBAD28} or E. coli^{pApyr}. Numbers indicate the percentage of positive cells within the displayed marker. (B) Statistical analysis of the frequency of Ki-67⁺ cells in ileal PPs among TCRβ⁺CD8⁺ cells in the indicated mice. Two-tailed Mann-Whitney U test. *p<0.05, **p<0.01.

FIG. 36 shows for Example 18 that administration of E. coli^{pApyr} results in increase of T-bet⁺ cells among CD8⁺ T cells in the Peyer's patches of the ileum. (A) Representative flow cytometry histograms of electronically gated TCRβ⁺CD8⁺ cells for T-bet expression in mice treated with anti-PD-L1, anti-PD-L1 and E. coli^{pBAD28} or E. coli^{pApyr}. Numbers indicate the percentage of positive cells within the displayed marker. (B) Statistical analysis of the frequency of T-bet⁺ cells in ileal PPs among TCRβ⁺CD8⁺ cells in the indicated mice. Two-tailed Mann-Whitney U test. ** p<0.01, *** p<0.001.

FIG. 37 shows for Example 19 the map of the pApyr plasmid carrying the phoN2 gene encoding apyrase used to transform Lactococcus lactis. PnisA, nisin A inducible promoter; SP usp45: signal sequence of usp45 gene; phoN2: S. flexneri apyrase gene; repC: replication gene C; repA: replication gene A; camR (cat): chloramphenicol resistance gene.

FIG. 38 shows for Example 20 that Lactococcus lactis^{pNZ-Apyr} improves anti-PD-L1 treatment outcome in mice bearing MC38 colon adenocarcinoma. Mice were inoculated s.c. with 1×10⁶ MC38 colon adenocarcinoma cells and on day 8, 11, 14 and 17 after tumor inoculation treated with PBS (MC38) or 100 μg of anti-PD-L1 antibody in 100 μl PBS i.p. Mice were also gavaged everyday with 1×10¹⁰ of L. lactis^{pNZ-Apyr} or transformants with empty vector L. lactis^{pNZ} (as indicated) or PBS from day 5 until the end of the experiment. Tumor growth was monitored until the experimental endpoint. Two-way ANOVA for the statistical analysis of tumor growth was applied. n=9 (MC38); 6 (MC38+aPDL1); 17 (MC38+aPDL1+L. lactis^{pNZ}); 20 (MC38+aPDL1+L. lactis^{pNZ-Apyr}). *p<0.05, **p<0.01, ***p<0.001, ****p<0.0001.

FIG. 39 shows for Example 21 the DNA fragment insertion for the integration of S. flexneri phoN2 gene in EcN genome. malP: EcN gene for maltodextrin phosphorylase; cat: E. coli gene for chloramphenicol acetyltransferase; phoN2: S. flexneri gene for apyrase; malT: EcN gene for the transcriptional activator of the maltose and maltodextrins operon; FRT: Flippase Recognition Target sequence; P cat: promoter of the cat gene; P_{proD}: promoter of the phoN2 gene; BBa_BB0032 RBS: Ribosome Binding Site of the phoN2 gene; T_{phoN2}: transcriptional terminator of the phoN2 gene.

FIG. 40 shows for Example 21 the nucleotide sequence of the EcN malP gene portion (SEQ ID NO: 4). The malP stop codon is indicated in bold.

FIG. 41 shows for Example 21 the nucleotide sequence of the EcN malT gene portion (SEQ ID NO: 5). The malT start codon is indicated in bold.

FIG. 42 shows for Example 21 the nucleotide sequence of the DNA fragment including the P_{proD} promoter, the BBa_BB0032 RBS, the S. flexneri phoN2 gene and the phoN2 transcriptional terminator (SEQ ID NO: 6). The P_{proD} sequence is underlined. The BBa_BB0032 RBS is shown in italics. The phoN2 start and stop codons are indicated in bold. The phoN2 transcriptional terminator is shown in bold italics.

FIG. 43 shows for Example 21 the nucleotide sequence of the DNA fragment including the E. coli cat gene flanked by the FRT sequences (SEQ ID NO: 7). The cat start and stop codons are indicated in bold. The FRT sequences are shown in italics.

FIG. 44 shows for Example 21 the malP-phoN2-malT recombinant genomic region of EcN::phoN2. malP: EcN gene for maltodextrin phosphorylase; phoN2: S. flexneri gene for apyrase; malT: EcN gene for the transcriptional activator of the maltose and maltodextrins operon; FRT: Flippase Recognition Target sequence; P_{proD}: promoter of the phoN2 gene; BBa_BB0032 RBS: Ribosome Binding Site of the phoN2 gene; T_{phoN2}: transcriptional terminator of the phoN2 gene.

FIG. 45 shows for Example 21 apyrase detection in EcN::phoN2 periplasmic extracts. EcN and EcN::phoN2 clone 1 (cl 1) bacterial cultures were grown for 2.5 h, in LB medium, at 37° C. and harvested by centrifugation. The periplasmic fraction of each culture was isolated, precipitated with trichloroacetic acid (TCA), solubilized in Laemmli buffer and analyzed by Western blot using a polyclonal anti-apyrase rabbit serum.

FIG. 46 shows for Example 21 the dose-dependent degradation of ATP by EcN::phoN2 periplasmic extract. EcN and EcN::phoN2 clone 1 (cl 1) bacterial cultures were grown for 6 h, in LB medium, at 37° C. and harvested by centrifugation. The periplasmic fraction of each culture was isolated, dialyzed against PBS 1× and serially diluted with PBS 1×. The apyrase activity in periplasmic extracts (PE) was measured as percentage of degradation of 50 μM ATP relative to PBS 1×. Apyrase activity in PE was evaluated by an ATP-dependent bioluminescence assay with recombinant firefly luciferase and its substrate D-luciferin according to the manufacturer's protocol (Life Technologies Europe B.V.).

FIG. 47 shows for Example 22 that E. coli Nissle 1917::phoN2 improves anti-PD-L1 treatment outcome in mice bearing MC38 colon adenocarcinoma. Mice were inoculated s.c. with 1×10⁶ MC38 colon adenocarcinoma cells and on day 8, 11, 14 and 17 after tumor inoculation treated with PBS (MC38) or 100 μg of anti-PD-L1 antibody in 100 μl PBS i.p. Mice were also gavaged everyday with 1×10¹⁰ of E. coli Nissle 1917 (EcN) or E. coli Nissle 1917 with phoN2 gene integrated in the genome (EcN::phoN2) or PBS from day 5 until the end of the experiment. Tumor growth was monitored until the experimental endpoint. Two-way ANOVA for the statistical analysis of tumor growth was applied. n=5 (MC38); 7 (MC38+aPD-L1+EcN); 6 (MC38+aPD-L1+EcN::phoN2). *p<0.05, **p<0.01, ***p<0.001.

FIG. 48 shows for Example 23 a schematic representation of the pBAD-OVA plasmid. pBAD, arabinose-inducible promoter; ova: cDNA encoding chicken ovalbumin; araC: arabinose operon regulator gene; f1 ori: f1 bacteriophage origin of replication; pBR322 ori: pBR322 plasmid origin of replication; kanR: kanamycin resistance gene.

FIG. 49 shows for Example 23 the nucleotide sequence of the cDNA encoding chicken ovalbumin used for the generation of the pBAD-OVA plasmid (SEQ ID NO: 8).

FIG. 50 shows for Example 23 the amino acid sequence of the chicken ovalbumin protein (SEQ ID NO: 9).

FIG. 51 shows for Example 24 that immunization with attenuated Salmonella Thypimurium^{pApyr-OVA} improves anti-PD-L1 treatment outcome in mice bearing MC38-OVA colon adenocarcinoma. Mice were inoculated s.c. with 1×10⁶ MC38 colon adenocarcinoma transfected with ovalbumin (MC38-OVA) and immunized by oral gavage with 1×10⁹ OVA-expressing Salmonella ThypimuriumPBAD-OVA (S. Tm^{pBAD-OVA}) or Apyrase/OVA-expressing Salmonella Thypimurium^{pApyr-OVA} (S. Tm^{pApyr-OVA}) at day 5 and 10 after tumor engraftment. On day 8, 11 and 14 after tumor inoculation, mice were treated i.p. with 100 μg of anti-PD-L1 antibody in 100 μl PBS i.p. The presence of the tumor was established at day 17. Chi-square test for the statistical analysis of tumor rejection was applied. N=9 (S. Tm^{pBAD-OVA}); 9 (S. Tm^{pApyr-OVA}). *p<0.05.

FIG. 52 shows for Example 25 that blockade of T cells egress from lymphoid organs abolishes the improvement of treatment outcome by E. coli^{pApyr} in mice bearing MC38 colon adenocarcinoma. Mice were inoculated s.c. with 1×10⁶ MC38 colon adenocarcinoma cells. On day 7 after tumor inoculation, mice were treated i.p. with PBS or FTY720 at 1 mg/kg and on day 8, 11, 14 and 17 treated with PBS (MC38) or 100 μg of anti-PD-L1 antibody in 100 μl PBS i.p. Mice were also gavaged everyday with 1×10¹⁰ of E. coli^{pApyr} (as indicated) or PBS from day 5 until the end of the experiment. Tumor growth was monitored until the experimental endpoint. Two-way ANOVA for the statistical analysis of tumor growth was applied. n=10 (MC38); 15 (MC38+aPD-L1); 15 (MC38+aPD-L1+FTY720); 15 (MC38+aPD-L1+E. coli^{pApyr}); 17 (MC38+aPD-L1+E. coli^{pApyr}+FTY720). **p<0.01, ***p<0.001, ****p<0.0001.

FIG. 53 shows for Example 26 that blockade of T cells egress from lymphoid organs abolishes the increase of CCR9⁺ and ICOS⁺ cells among CD8⁺ TILs induced by administration of E. coli^{pApyr}. (Left) Statistical analysis of the frequency of CCR9⁺ cells among TCRβ⁺CD8⁺ TILs in mice bearing MC38 tumors and treated with anti-PD-L1 or anti-PD-L1 and FTY720. Mice were also gavaged everyday with 1×10¹⁰ of E. coli^{pApyr} (as indicated) or PBS from day 5 after tumor inoculation. (Right) Statistical analysis of the frequency of ICOS⁺ cells among TCRβ⁺CD8⁺ TILs in the same mice. Two-tailed Mann-Whitney U test. **p<0.01, ***p<0.001.

FIG. 54 shows for Example 27 that administration of E. coli^{pApyr} in mice bearing MC38 tumors and treated with anti-PD-L1 results in increase of IgA coating of the ileal microbiota. (A) Representative flow cytometry contour plots of bacteria electronically gated by staining with SYTO BC green fluorescent nucleic acid stain (Syto⁺) for side scatter (SSC-A) and IgA coating (IgA) revealed by anti-mouse IgA antibodies. Bacteria were isolated from the ileum of mice bearing MC38 tumors, treated with anti-PD-L1 (Ctrl) or anti-PD-L1 and E. coli^{pBAD28} (+E. coli^{pBAD28}) or anti-PD-L1 and E. coli^{pApyr} (+E. coli^{pApyr}) at the end of the experiment. Numbers indicate the percentage of positive cells in the displayed quadrant. (B) Statistical analysis of the frequency of IgA bound bacteria in the ileum of the indicated mice. Two-tailed Mann-Whitney U test. *** p<0.001.

FIG. 55 shows for Example 28 that the increase of Ki-67⁺ cells among CD8⁺ T cells in the Peyer's patches by administration of E. coli^{pApyr} depends on IgA. (Left) Representative flow cytometry histograms of electronically gated TCRβ⁺CD8⁺ cells for Ki-67 expression in Peyer's patches from wild-type and IgA^{-/-} C57Bl/6 mice treated with anti-PD-L1 (Ctrl) or anti-PD-L1 and E. coli^{pApyr} (+E. coli^{pApyr}). Numbers indicate the percentage of positive cells within the displayed marker. (Right) Statistical analysis of the frequency of Ki-67⁺ cells in ileal PPs among TCRβ⁺CD8⁺ cells in the indicated mice. Two-tailed Mann-Whitney U test. *p<0.05.

FIG. 56 shows for Example 29 that the increase of T-bet⁺ cells among CD8⁺ T cells in the Peyer's patches by administration of E. coli^{pApyr} depends on IgA. (Left) Representative flow cytometry histograms of electronically gated TCRβ⁺CD8⁺ cells for T-bet expression in Peyer's patches from wild-type and IgA^{-/-} C57Bl/6 mice treated with anti-PD-L1 (Ctrl) or anti-PD-L1 and E. coli^{pApyr} (+E. coli^{pApyr}). Numbers indicate the percentage of positive cells within the displayed marker. (Right) Statistical analysis of the frequency of Ki-67⁺ cells in ileal PPs among TCRβ⁺CD8⁺ cells in the indicated mice. Two-tailed Mann-Whitney U test. *p<0.05.

FIG. 57 shows for Example 30 that E. coli^{pApyr} does not improve the anti-PD-L1 treatment outcome in IgA^{-/-} mice bearing MC38 colon adenocarcinoma. Wild-type and IgA-C57Bl/6 mice were inoculated s.c. with 1×10⁶ MC38 colon adenocarcinoma cells and on day 8, 11, 14 and 17 after tumor inoculation treated with PBS (MC38) or 100 μg of anti-PD-L1 antibody in 100 μl PBS i.p. Mice were also gavaged everyday with 1×10¹⁰ of E. coli^{pApyr} (as indicated) or PBS from day 5 until the end of the experiment. Tumor growth was monitored until the experimental endpoint. Two-way ANOVA for the statistical analysis of tumor growth was applied. n=3 (MC38); 7 (MC38 in IgA^{-/-}); 7 (MC38+aPDL1+E. coli^{pApyr}); 11 (MC38 in IgA^{-/-}+aPDL1+E. coli^{pApyr}). *p<0.05, **p<0.01, ***p<0.001.

FIG. 58 shows for Example 31 the lack of increase of CCR9⁺ and ICOS⁺ cells among CD8⁺ TILs by administration of E. coli^{pApyr} in mice lacking IgA. (Left) Statistical analysis of the frequency of CCR9⁺ cells among TCRβ⁺CD8⁺ TILs in wildtype and IgA^{-/-} C57Bl/6 mice bearing MC38 tumors and treated with anti-PD-L1. Mice were also gavaged everyday with 1×10¹⁰ of E. coli^{pApyr} (as indicated) or PBS from day 5 after tumor inoculation. (Right) Statistical analysis of the frequency of ICOS⁺ cells among TCRβ⁺CD8⁺ TILs in the same mice. Two-tailed Mann-Whitney U test. *p<0.05.

FIG. 59 shows for Example 32 that the frequency of IgA coated bacteria in the ileum correlates with the tumor size in mice bearing MC38 colon adenocarcinoma and treated with anti-PD-L1. Mice were inoculated s.c. with 1×10⁶ MC38 colon adenocarcinoma cells and on day 8, 11, 14 and 18 after tumor inoculation treated with 100 μg of anti-PD-L1 antibody in 100 μl PBS i.p. Mice were also gavaged everyday with 1×10¹⁰ of E. coli^{pBAD28} (black circles) or E. coli^{pApyr} (grey squares) from day 5 until the end of the experiment. Correlation between tumor size and the percentage of IgA coated bacteria in the ileum at day 20 after tumor engraftment. The correlation coefficient r and the respective P value were calculated with nonparametric Spearman test. Each dot in graphs represents an individual mouse.

FIG. 60 shows for Example 32 that the frequency of IgA coated bacteria in the ileum correlates with tumor size in mice bearing MC38 colon adenocarcinoma and treated with anti-PD-L1. Mice were inoculated s.c. with 1×10⁶ MC38 colon adenocarcinoma cells and on day 8, 11, 14 and 17 after tumor inoculation treated with 100 μg of anti-PD-L1 antibody in 100 μl PBS i.p. Mice were also gavaged everyday with 1×10¹⁰ of E. coli Nissle 1917 (EcN) (black circles) or EcN with chromosome integrated apyrase encoding gene (phoN2) from Shigella flexneri (Ecn: phoN2) (grey squares) from day 5 until the end of the experiment. Correlation between tumor size and the percentage of IgA coated bacteria in the ileum at day 18 after tumor engraftment. The correlation coefficient r and the respective P value were calculated with nonparametric Spearman test. Each dot in graphs represents an individual mouse.

FIG. 61 shows for Example 33 that administration of Vancomycin abolishes the improvement of treatment outcome by E. coli^{pApyr} in mice bearing MC38 colon adenocarcinoma. Mice were treated with Vancomycin (200 mg/L) in drinking water for 15 days (as indicated) and inoculated s.c. with 1×10⁶ MC38 colon adenocarcinoma cells (day 0). Vancomycin was maintained in the drinking water until the end of the experiment. On day 8, 11, 14 and 17 mice were treated with PBS (MC38 and MC38+Vancomycin) or 100 μg of anti-PD-L1 antibody in 100 μl PBS i.p. Mice were also gavaged everyday with 1×10¹⁰ of E. coli^{pBAD28} or E. coli^{pApyr} (as indicated) or PBS from day 5 until the end of the experiment. Tumor growth was monitored until the experimental endpoint. Two-way ANOVA for the statistical analysis of tumor growth was applied. n=4 (MC38); 4 (MC38+Vancomycin); 5 (MC38+aPD-L1+E. coli^{pBAD28}); 7 (MC38+aPD-L1+E. coli^{pBAD28}+Vancomycin); 7 (MC38+aPD-L1+E. coli^{pApyr}); 8 (MC38+aPD-L1+E. coli^{pApyr}+Vancomycin). *p<0.05, **p<0.01.

FIG. 62 shows for Example 34 that administration of Vancomycin affects IgA coated bacteria in the ileum of mice treated with E. coli^{pApyr}. (Left) Representative flow cytometry contour plots of bacteria electronically gated by staining with SYTO BC green fluorescent nucleic acid stain (Syto⁺) for side scatter (SSC-A) and IgA coating (IgA) revealed by anti-mouse IgA antibodies. Bacteria were isolated at the end of the experiment, from the ileum of mice bearing MC38 tumors, treated with anti-PD-L1 and E. coli^{pBAD28} (+E. coli^{pBAD28}) or anti-PD-L1 and E. coli^{pApyr} (+E. coli^{pApyr}) either in the presence or not of Vancomyin in the drinking water (as indicated). Numbers indicate the percentage of positive cells in the displayed quadrant. (Right) Statistical analysis of the frequency of IgA bound bacteria in the ileum of the indicated mice. Two-tailed Mann-Whitney U test. *p<0.05, **p<0.01.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Design and Production of Apyrase-Expressing Bacteria

To obtain bacteria expressing apyrase, full length phoN2::HA fusion, encoding periplasmic ATP-diphosphohydrolase (apyrase) of Shigella flexneri (SEQ ID NO: 1) with a hemagglutinin (HA) fragment as tag were cloned into the polylinker site of plasmid pBAD28 (ATCC 8739387402), under the control of the PBAD L-arabinose inducible promoter. Thereby, plasmid pHND10 was generated, essentially as described in Santapaola, D., Del Chierico, F., Petrucca, A., Uzzau, S., Casalino, M., Colonna, B., Sessa, R., Berlutti, F., and Nicoletti, M. (2006). Apyrase, the product of the virulence plasmid-encoded phoN2 (apy) gene, is necessary for proper unipolar IcsA localization and for efficient intercellular spread. Journal of bacteriology 188, p. 1620-1627.

As control, plasmid pHND19 was produced essentially as described in Scribano, D., Petrucca, A., Pompili, M., Ambrosi, C., Bruni, E., Zagaglia, C., Prosseda, G., Nencioni, L., Casalino, M., Polticelli, F., et al. (2014). Polar localization of PhoN2, a periplasmic virulence-associated factor of Shigella flexneri, is required for proper IcsA exposition at the old bacterial pole. PloS one 9, e90230. In contrast to the pHND10 plasmid, the pHND19 plasmid (control) contains a phoN2_{R192}P::HA fusion, which encodes a loss-of-function isoform of apyrase carrying the R192P substitution.

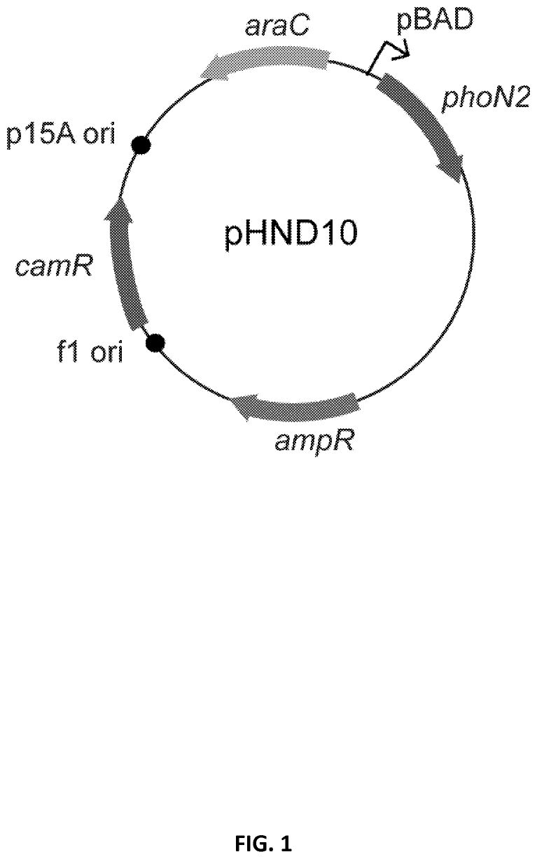

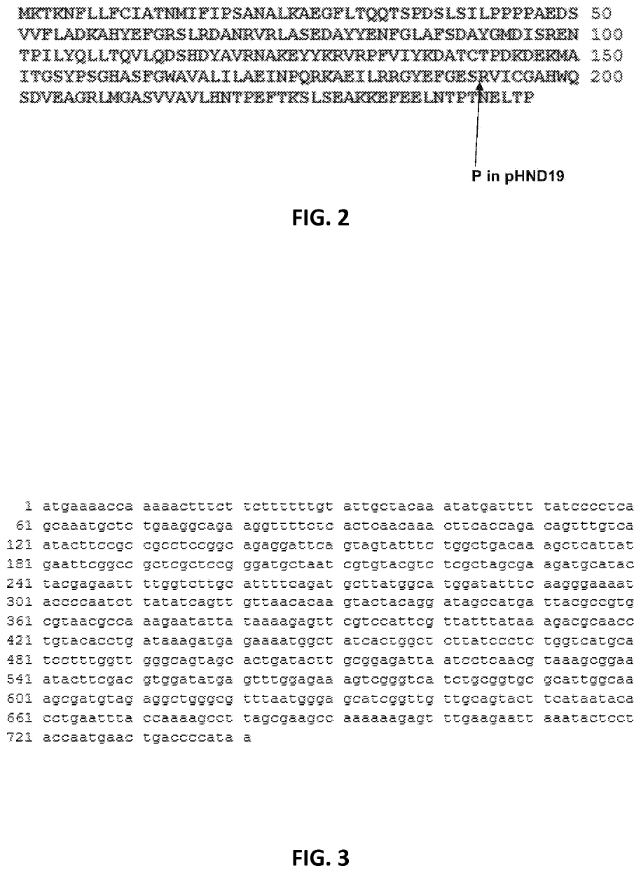

FIG. 1 shows a map of the pHND10 plasmid carrying the phoN2 gene encoding periplasmic ATP-diphosphohydrolase (apyrase). This map applies in general also to the pHND19 control plasmid, with the only difference that the loss-of-function isoform of apyrase carrying the R192P substitution is encoded instead of wild-type apyrase. FIG. 2 shows the amino acid sequence of wild-type phon2 protein (apyrase; SEQ ID NO: 1) and indicates the position of the R192P substitution in the loss-of-function isoform (SEQ ID NO: 2). The nucleotide sequence of the phoN2 gene used for generating pHND10 plasmid is shown in FIG. 3 (SEQ ID NO: 3).

Escherichia coli DH10B were transformed with pHND10 (E. coli^{pApyr}) or pHND19R192P (E. coli^{pHND19}) and grown in LB medium supplemented with L-arabinose (0.03%) and ampicillin (100 μg/ml).

### Example 2: Bacteria Expressing Apyrase Improve Anti-PD-L1 Treatment of Melanoma

To investigate the effect of administration of bacteria expressing apyrase (obtained as described in Example 1) in combination with an immune checkpoint inhibitor on melanoma treatment, B16F10 melanoma cells transfected with ovalbumin (B16-OVA) were grafted subcutaneously into C57BL/6 mice to mimic the expression of a tumor neo-antigen, essentially as described in Bellone, M., Cantarella, D., Castiglioni, P., Crosti, M. C., Ronchetti, A., Moro, M., Garancini, M. P., Casorati, G., and Dellabona, P. (2000). Relevance of the tumor antigen in the validation of three vaccination strategies for melanoma. J Immunol 165, 2651-2656. Briefly, ovalbumin-expressing melanoma B16F10 (B16-OVA) cells were cultured in RPMI-1640 supplemented with 10% heat-inactivate fetal bovine serum, 100 U/mL penicillin/streptomycin and 100 U/mL kanamycin. Cells were maintained in 5% CO₂ at 37° C. Tumor cells were harvested at exponential growth and subcutaneously engrafted in 8 week old C57Bl/6 mice at 1×10⁶ cells/100 μl (day 0).

Mice were orally gavaged with E. coli expressing either apyrase (E. coli^{pApyr}) or the loss of function isoform of the enzyme with R192P amino acid substitution (E. coli^{pHND}19), as described in Example 1, in combination with intra-peritoneal administration of anti-PD-L1. Mice were injected intraperitoneally with anti-PD-L1 monoclonal antibody (clone: 10F.9G2; BioXCell) (100 μg/100 μl) at day 8, 11, 14, 18. The indicated Escherichia coli transformants (1×10¹⁰ CFU) were administered daily by orogastric gavage from day 5 to termination of the experiment. Tumor growth was scored with a caliper by measuring the greatest tumor diameter and its perpendicular to determine an average and then the area was calculated as: (average/2)²π.

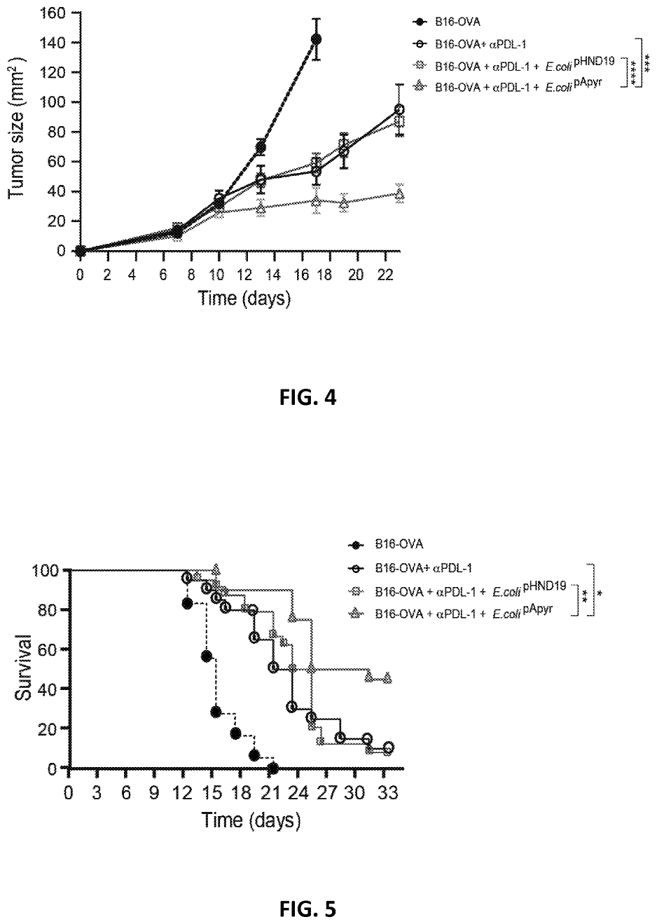

The results are shown in FIG. 4. Surprisingly, these experiments showed a significant reduction of tumor growth in mice treated with a combination of anti-PD-L1 and E. coli^{pApyr} as compared to the groups treated with anti-PD-L1 alone or in combination with E. coli transformed with the control plasmid (E. coli^{pHND19}). Without being bound to any theory, the present inventors assume that apyrase enzymatic activity conditioned the intestinal ecosystem during therapy with immune checkpoint inhibitors and improved the generation of a proficient anti-tumor response.

Survival rates of the mice are shown in FIG. 5. Analysis of survival of mice following B16-OVA tumor engraftment revealed significantly enhanced survival in mice treated with a combination of anti-PD-L1 and E. coli^{pApyr} as compared to the groups treated with anti-PD-L1 alone or in combination with control E. coli^{pHND19}.

### Example 3: Bacteria Expressing Apyrase Improve Anti-PD-L1 Treatment of Colon Adenocarcinoma

To investigate the effect of administration of bacteria expressing apyrase (obtained as described in Example 1) in combination with an immune checkpoint inhibitor on a distinct tumor model, colon adenocarcinoma MC38 cells were grafted subcutaneously into C57BL/6 mice.

The experiments were performed essentially as described in Example 2, with the difference that distinct tumor cells (MC38 colon adenocarcinoma cells) were used. Briefly, colon adenocarcinoma MC38 cells were cultured in RPMI-1640 supplemented with 10% heat-inactivate fetal bovine serum, 100 U/mL penicillin/streptomycin and 100 U/mL kanamycin. Cells were maintained in 5% CO₂ at 37° C. Tumor cells were harvested at exponential growth and subcutaneously engrafted in 8 week old C57Bl/6 mice at 1×10⁶ cells/100 μl (day 0).

Similarly as in Example 2, mice were orally gavaged with E. coli expressing either apyrase (E. coli^{pApyr}) or the loss of function isoform of the enzyme with R192P amino acid substitution (E. coli^{pHND19}), as described in Example 1, in combination with intra-peritoneal administration of anti-PD-L1. Mice were injected intraperitoneally with anti-PD-L1 monoclonal antibody (clone: 10F.9G2; BioXCell) (100 μg/100 μl) at day 8, 11, 14, 18. The indicated Escherichia coli transformants (1×10¹⁰ CFU) were administered daily by orogastric gavage from day 5 to termination of the experiment. Tumor growth was scored with a caliper by measuring the greatest tumor diameter and its perpendicular to determine an average and then the area was calculated as: (average/2)²π.

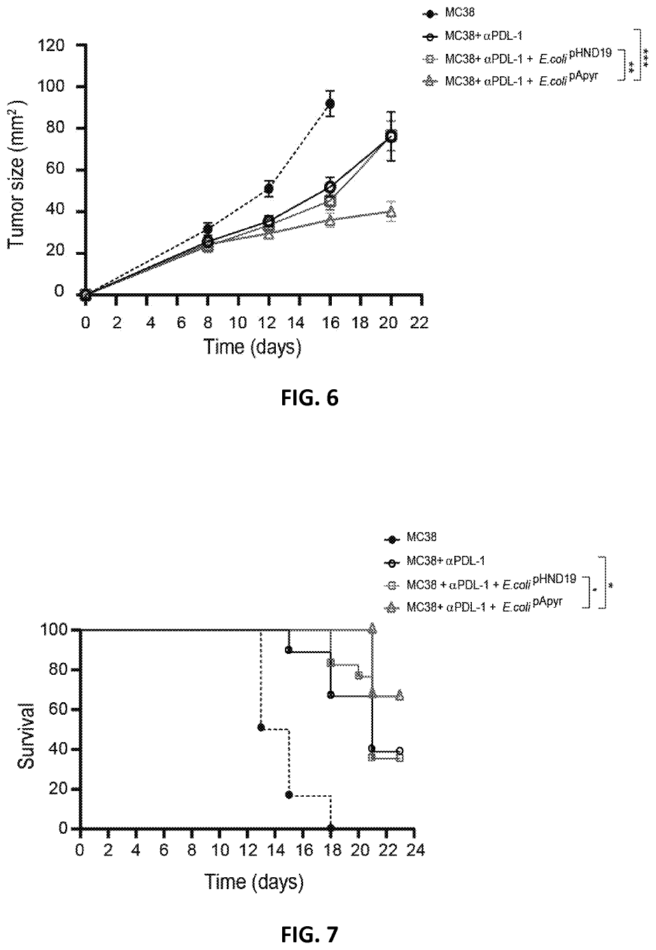

Results are shown in FIG. 6. Similarly as in Example 2, a significant reduction of tumor growth in mice treated with a combination of anti-PD-L1 and E. coli^{pApyr} was observed as compared to the groups treated with anti-PD-L1 alone or in combination with E. coli transformed with the control plasmid (E. coli^{pHND19}).

Survival rates of the mice are shown in FIG. 5. Analysis of survival of mice following the engraftment of MC38 tumor revealed significantly enhanced survival in mice treated with a combination of anti-PD-L1 and E. coli^{pApyr} as compared to the groups treated with anti-PD-L1 alone or in combination with control E. coli^{pHND19}, thus confirming that administration of apyrase expressing bacteria improves efficacy of treatment with immune checkpoint inhibitors.

### Example 4: Probiotic Bacteria Expressing Apyrase Improve Anti-PD-L1 Treatment of Tumors

To investigate apyrase delivery by a probiotic microorganism, probiotic bacteria of the strain Escherichia coli Nissle 1917 expressing wild-type apyrase were obtained essentially as described in Example 1. Briefly, Escherichia coli Nissle 1917 were transformed with pHND10 (Nissle^{pApyr}) and grown in LB medium supplemented with L-arabinose (0.03%) and ampicillin (100 μg/ml), as described in Example 1.

Probiotic strain Escherichia coli Nissle 1917 expressing apyrase (Nissle^{pApyr}) were investigated in the MC38 tumor model as described in Example 3, i.e. in combination with anti-PD-L1 antibodies to mice bearing MC38 tumours, and compared to an untreated MC38 control group, an MC38 control group receiving anti-PDL-1 only and an MC38 control group receiving anti-PDL-1 and E. coli Nissle 1917 (without pHND10 for apyrase expression).

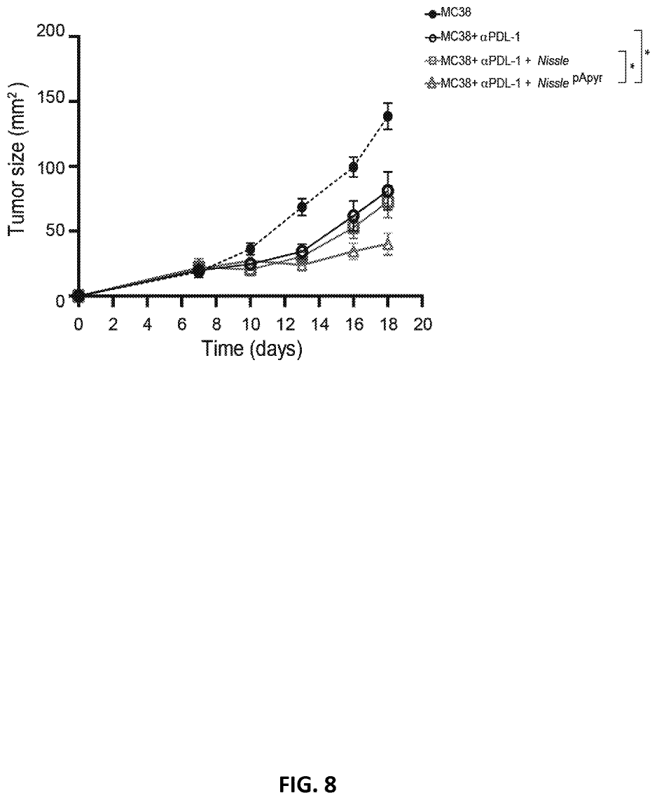

Results are shown in FIG. 8. No amelioration of the therapeutic effect of anti-PD-L1 alone was observed. However, expression of phoN2 in E. coli Nissle 1917 resulted in a significant inhibition of tumour growth with respect to mice treated with anti-PD-L1 or anti-PD-L1 and E. coli Nissle 1917. This result shows that apyrase delivery via a probiotic microorganism ameliorates the outcome of cancer immunotherapy.

### Example 5: Administration of a Composition Comprising Apyrase Improves Anti-PD-L1 Treatment in a Tumor Model

To investigate whether administration of live bacteria expressing apyrase was a requirement for the effects observed in above Examples 2-4, or whether administration of apyrase would be sufficient, administration of a composition comprising apyrase, namely, a periplasmic extract from E. coli^{pApyr}, was investigated in the MC38 tumor model as described above.

To prepare periplasmic extract, E. coli^{pApyr} were obtained and grown as described above (see Example 1) and collected by centrifugation. After washing, bacteria were resuspended (10¹⁰ CFU/ml) in PBS with 30 mM Tris-HCl (pH 8.0), 4 mM EDTA, 1 mM PMSF, 20% sucrose and 0.5 mg/ml lysozyme and incubated 2 min at 30° C. MgCl₂ (10 mM final) was added to the bacterial solution and incubation was continued for 1 h at 30° C. At the end of the incubation period bacterial suspensions were centrifuged at 11,000×g for 10 min at 4° C. and supernatants were stored (periplasmic extract).

Colon adenocarcinoma MC38 cells were cultured in RPMI-1640 supplemented with 10% heat-inactivate fetal bovine serum, 100 U/mL penicillin/streptomycin and 100 U/mL kanamycin. Cells were maintained in 5% CO₂ at 37° C. Tumor cells were harvested at exponential growth and subcutaneously engrafted in 8 wk old C57Bl/6 mice at 1×10⁶ cells/100 μl (day 0). Mice were injected intra-peritoneally with anti-PD-L1 monoclonal antibody (clone: 10F.9G2; BioXCell) (100 μg/100 μl) at day 8, 11, 14, 18. Either 100 μl of periplasmic extract or E. coli^{pApyr} (1×10¹⁰ CFU) were administered daily by orogastric gavage from day 5 to termination of the experiment. Tumor growth was scored with a caliper by measuring the greatest tumor diameter and its perpendicular to determine an average and then the area was calculated as: (average/2)²π.

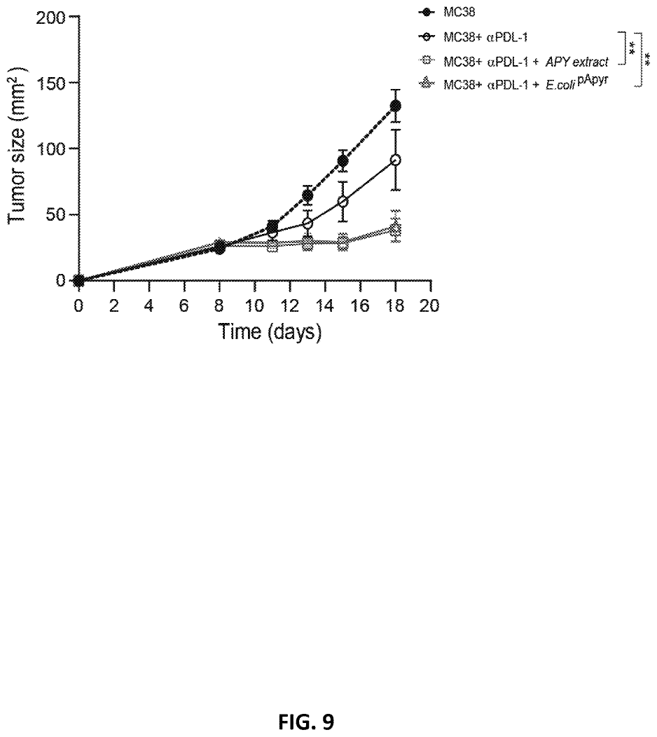

Results are shown in FIG. 9. Similarly as in Examples 2 and 3, a significant reduction of tumor growth in mice treated with a combination of anti-PD-L1 and E. coli^{pApyr} was observed as compared to mice treated with anti-PD-L1 alone. Combination of anti-PD-L1 and a composition comprising apyrase, namely the periplasmic extract as described above, resulted in the same inhibition of tumor growth as observed in mice treated with live E. coli^{pApyr}. This shows that administration of the apyrase protein is sufficient to increase the therapeutic efficacy of treatment with immune checkpoint inhibitors.

### Example 6: Administration of E. coli^{pApyr} Results in Increase of CXCR5⁺ Cells Among CD8⁺ TILS

To analyze the immunophenotype of tumor infiltrating lymphocytes (TILs) in MC38 tumors from C57Bl/6 mice, the neoplastic tissue of mice of Example 3 was digested and leukocytes were enriched. To this end, tumors were cut in small pieces and resuspended in RPMI-1640 with 1.5 mg/ml type I collagenase (Sigma), 100 μg/mL DNase I (Roche) and 5% FBS, digested for 45 min at 37° C. under gentle agitation. The digestion product was then passed through a 70 μm cell strainer to obtain a single cell suspension. Lymphocytes were then enriched by Percoll density gradient following manufacturer's protocol.

CD8⁺ TILs were analysed in flow cytometry by staining with various fluorescently labelled antibodies together with CD8 and TCRβ chain specific antibodies to electronically gate CD8⁺ TILs. Briefly, cells were stained with the following monoclonal antibodies: biotin-conjugated anti-CXCR5 (clone: 2G8; BD), PE-labeled anti-ICOS (clone: 7E.17G9; BD), AF488-labeled anti-TCRβ (clone: H57-597; BioLegend), APC-labeled or APCy7-labeled anti-CD8α (clone: 53-6.7; eBioscience,) PeCy7-labeled anti-CD25 (clone: PC61; BioLegend), AF-647-labeled anti-TCF1 (clone 7F11A10; Biolegend), PECy7-labeled anti-CD11c (clone: N418; BioLegend), AF405-labeled anti-MHC class II (clone: M5/114.15.2; BioLegend), biotin-conjugated anti-CD70 (clone: FR70; eBioscience) and PE-labeled anti-CD103 (clone: 2E7; BioLegend). FITC-labeled streptavidin was purchased from BioLegend and efluo405-labeled streptavidin from eBioscience. Intracellular staining was performed using the BD Cytofix/Cytoperm and Perm/Wash buffers or, for intracellular FoxP3 (FITC-labeled, clone: FJK-16s; eBioscience) staining, the eBioscience FoxP3 staining buffer set. Samples were acquired on a LSRFortessa (BD Bioscience) flow cytometer. Data were analyzed using FlowJo software (TreeStar) or FACS Diva software (BD Bioscience).

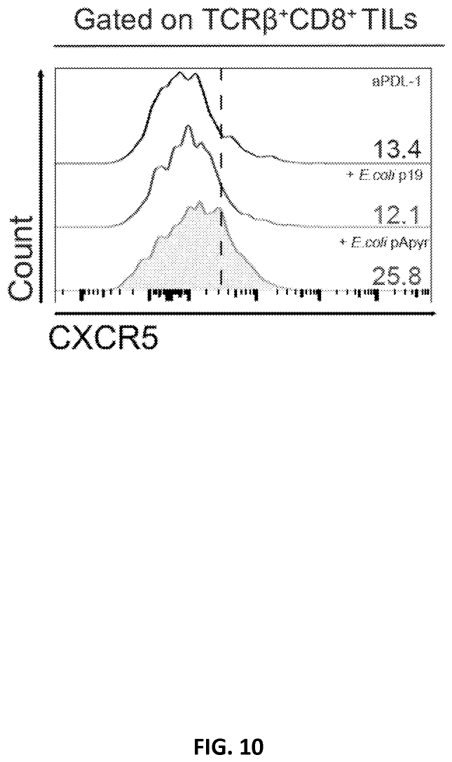

The analysis of cells stained with anti-CXCR5 antibodies surprisingly revealed the increase of CXCR5⁺CD8⁺ TILs in tumors from mice treated with a combination of anti-PD-L1 and E. coli^{pApyr} as compared to the groups treated with anti-PD-L1 alone or in combination with E. coli^{pHND19}. Results are shown in FIG. 10. Without being bound to any theory, the present inventors assume that the enrichment of this TILs component may have contributed to the improved control of tumor growth and better prognosis observed in mice treated with anti-PD-L1 combined with E. coli^{pApyr}.

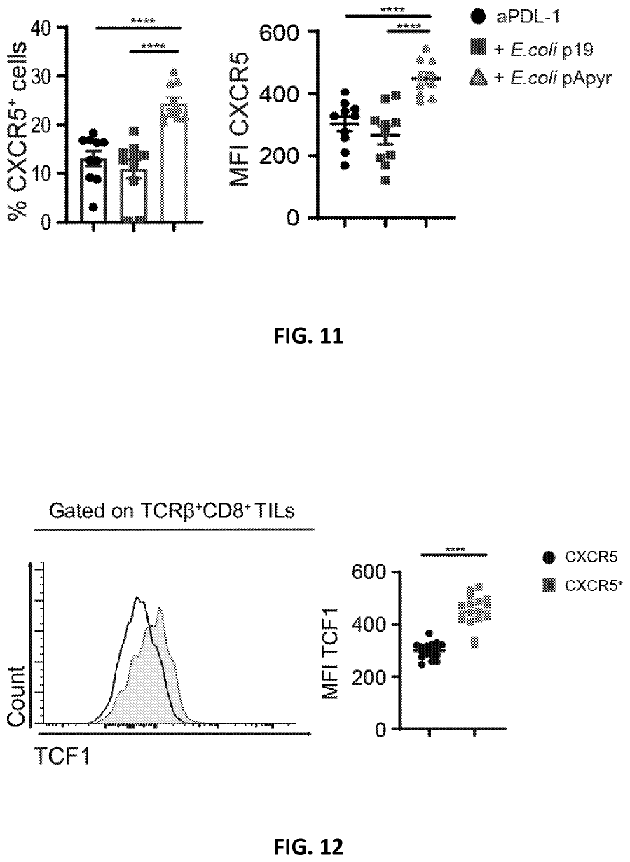

As shown in FIG. 11, Statistical analysis of the frequencies of CXCR5⁺CD8⁺ TILs in tumors from different animals showed the significant increase of these cells in mice treated with a combination of anti-PD-L1 and E. coli^{pApyr} as compared to the groups treated with anti-PD-L1 alone or in combination with E. coli^{pHND19}. Moreover, the analysis of the expression levels of CXCR5 in the plasma membrane of CD8 TILs in flow cytometry revealed the significant increase of the mean fluorescence intensity (MFI) in cells isolated from tumors that developed in mice treated with anti-PD-L1 in combination with E. coli^{pApyr} This indicates a positive regulation of CXCR5 protein expression by the combined administration of immune checkpoint inhibitors and apyrase expressing bacteria.

CXCR5⁺CD8⁺ cells are characterized by the expression of the transcription factor TCF1, a master regulator of T cell exhaustion that represses pro-exhaustion factors and induces Bcl6 in CD8⁺ T cells, thereby promoting stem cell-like self-renewal. Therefore, TCF1 expression was analyzed in CXCR5⁻ and CXCR5⁺ subsets of electronically gated CD8⁺ TILs in MC38 tumors. FIG. 12 shows representative flow cytometry histograms. TCF1 expression was found to be upregulated in CXCR5⁺CD8⁺ TILs that were expanded in MC38 tumor bearing mice treated with anti-PD-L1 and E. coli^{pApyr} with respect to CXCR5-CD8⁺ cells that dominate in the tumor microenvironment (TME) of untreated mice.

### Example 7: Administration of E. coli^{pApyr} Results in Increase of CXCR5⁺ Cells Among CD8⁺ T Cells in Peyer's Patches of the Ileum

Next, it was investigated whether E. coli^{pApyr} administration affected CXCR5⁺CD8⁺ cells in the Peyer's patches (PPs) of the small intestine, where T cell mediated immune responses are conditioned by the intestinal ecosystem. Peyer's patches (PPs) are the secondary lymphoid organs within the ileal mucosa, where T cell dependent IgA responses originate. Most lymphocytes localized in PPs inhabit germinal centers (GCs), where T follicular helper (Tfh) cells interact with B cells and facilitate B cell proliferation, induction of activation-induced (cytidine) deaminase (AID) with consequent Ig class switch recombination (CSR), somatic hyper mutation (SHM) and affinity maturation (Crotty, S. (2011). Follicular helper CD4 T cells (TFH). Annual review of immunology 29, 621-663). Since Tfh cells in PPs are essential for GC reaction and IgA affinity maturation, they play a critical role in the modulation of the structure and function of intestinal microbial communities (Kawamoto, S., Maruya, M., Kato, L. M., Suda, W., Atarashi, K., Doi, Y., Tsutsui, Y., Qin, H., Honda, K., Okada, T., et al. (2014). Foxp3 (+) T cells regulate immunoglobulin A selection and facilitate diversification of bacterial species responsible for immune homeostasis. Immunity 41, 152-165).

To this end, PPs of mice of Example 3 were digested, leukocytes were enriched and CD8⁺ T cells were analyzed in flow cytometry essentially as described in Example 6 for neoplastic tissue.

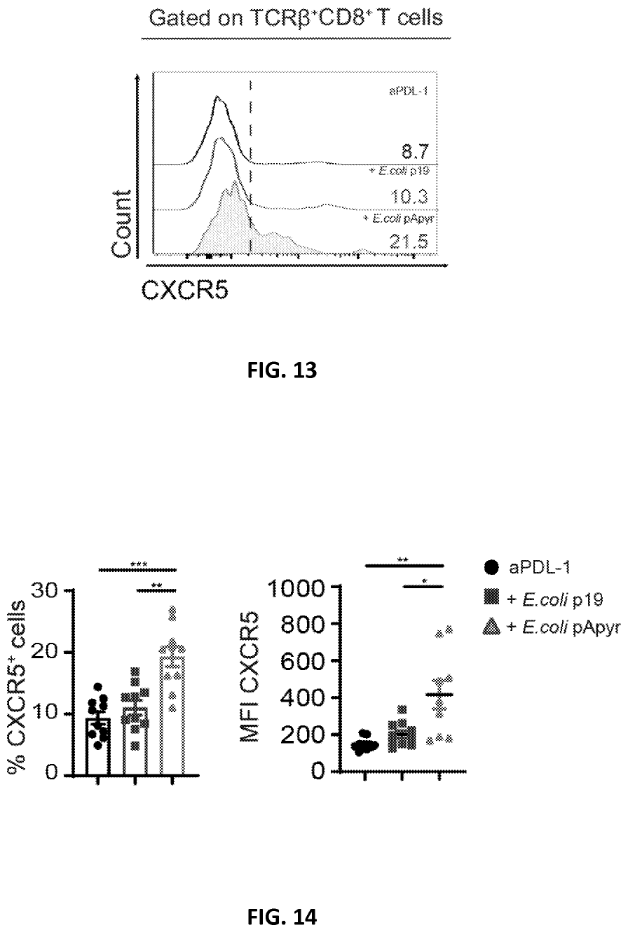

Results are shown in FIG. 13. Analogously to tumor tissue, CXCR5⁺CD8⁺ cells were increased in PPs from mice treated with anti-PD-L1 in combination with E. coli^{pApyr}, whereas the abundance of this cell population was similar in mice treated with anti-PD-L1 together with bacteria expressing the loss of function mutant of apyrase and mice receiving anti-PD-L1 without bacteria. This shows that administration of anti-PD-L1 in combination with E. coli^{pApyr} results in an increase of CXCR5⁺ cells among CD8⁺ T cells in the Peyer's patches of the ileum. Without being bound to any theory, the present inventors assume that apyrase mediated conditioning of the gut ecosystem results in induction of CXCR5⁺CD8⁺ cells in local secondary lymphoid organs that are constantly stimulated by microbiota derived antigens.

As shown in FIG. 14, statistical analysis of the frequencies of CXCR5⁺CD8⁺ T cells in PPs from different animals showed the significant increase of these cells in mice treated with a combination of anti-PD-L1 and E. coli^{pApyr} as compared to the groups treated with anti-PD-L1 alone or in combination with E. coli^{pHND19}. Moreover, the analysis of the expression levels of CXCR5 in the plasma membrane of CD8⁺ T cells in flow cytometry revealed the significant increase of the mean fluorescence intensity (MFI) in cells isolated from PPs of mice treated with anti-PD-L1 together with E. coli^{pApyr}. This indicates a positive regulation of CXCR5 protein expression by apyrase in CD8⁺ T cells from PPs.

### Example 8: Administration of E. coli^{pApyr} results in increase of ICOS⁺ cells among CD8⁺ TILs

ICOS expression was analyzed by flow cytometry on electronically gated CD8⁺ TILs as described above in Example 6.

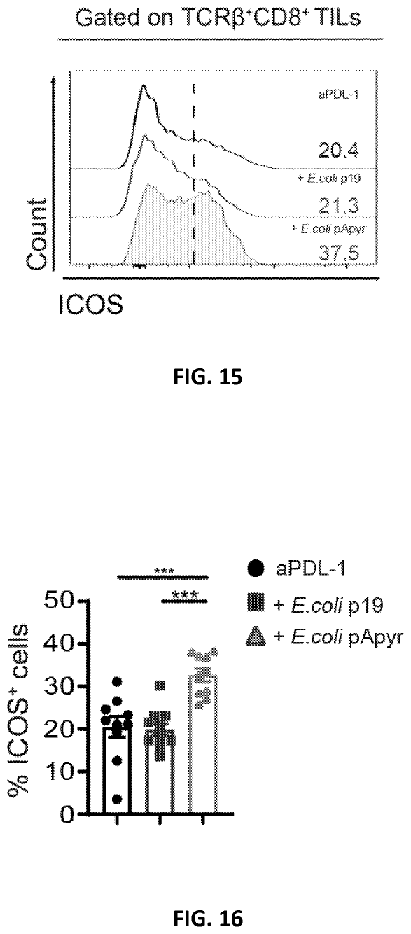

Results are shown in FIG. 15. Strikingly, TILs isolated from MC38 tumors resected from mice treated with a combination of anti-PD-L1 and E. coli^{pApyr} showed an increase of ICOS⁺ cells among electronically gated CD8⁺ TILs as compared to the mice treated with anti-PD-L1 alone or in combination with E. coli^{pHND19}.

As shown in FIG. 16, statistical analysis of the frequencies of ICOS⁺CD8⁺ TILs in tumors from different animals showed the significant increase of these cells in mice treated with a combination of anti-PD-L1 and E. coli^{pApyr} as compared to the groups treated with anti-PD-L1 alone or in combination with E. coli^{pHND19}.

### Example 9: Administration of E. coli^{pApyr} Results in Increase of IFN-γ Secreting Cells and IL-21 Secreting Cells Among CD8⁺ TILs

Next, it was tested whether the enhanced responsiveness to anti-PD-L1 administration by E. coli^{pApyr} was associated with increased secretion of IFN-γ or increased production of IL-21 by CD8⁺ TILs. To this end, IFN-γ and IL-21 secretion in CD8⁺ TILs from mice bearing MC38 tumors was analyzed essentially as described in Example 6. For intracellular staining of IL-21 (R&D Systems), IFN-γ (PeCy7-labeled, clone: XMG1.2; eBioscience), tumor infiltrating cells were cultured for 5 h at 37° C. in medium containing ionomycin (750 ng/ml) and PMA (20 ng/ml). For the last 4 h, Monensin (1000× Solution, eBioscience) was added to the cultures. IL-21 was detected with a recombinant mouse IL-21R subunit/human IgG1 Fc chimera (R&D Systems) with goat anti-human Fcγ conjugated to AF488 (Jackson ImmunoResearch).

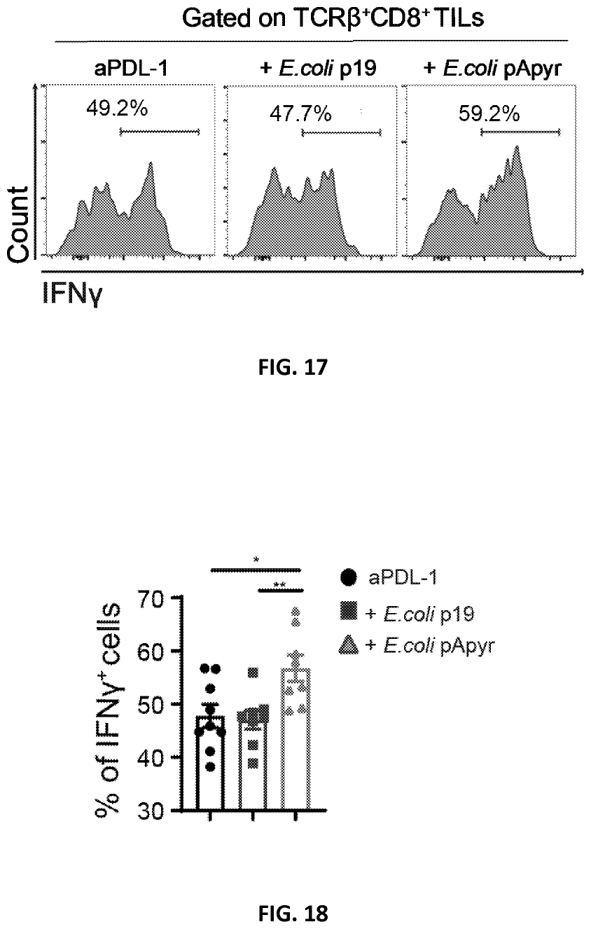

Results for IFN-γ analysis are shown in FIG. 17. The analysis of IFN-γ secretion in CD8⁺ TILs from mice bearing MC38 tumors and treated with anti-PD-L1 combined with E. coli^{pApyr} revealed enhanced frequencies of IFN-γ secreting cells in comparison to mice treated with anti-PD-L1 alone or in combination with E. coli^{pHND19}. As shown in FIG. 18, the statistical analysis of the frequencies of IFN-γ secreting CD8⁺ TILs in different animals showed the significant increase of these cells in mice treated with anti-PD-L1 and gavaged with E. coli^{pApyr} as compared to the groups treated with anti-PD-L1 alone or in combination with E. coli^{pHND19}.

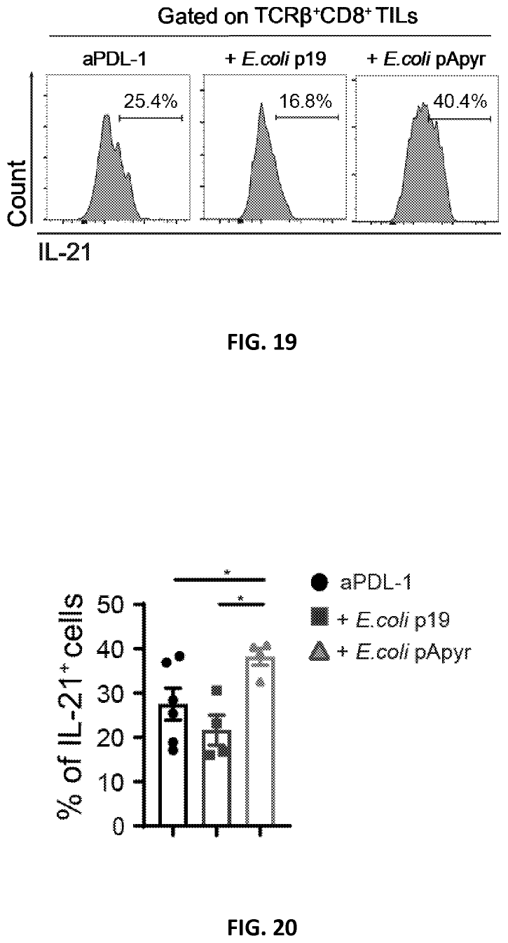

Results for IL-21 analysis are shown in FIG. 19. Surprisingly, administration of anti-PD-L1 combined with daily gavaging of E. coli^{pApyr} resulted in a robust increase in the frequency of IL-21 secreting cells among CD8⁺ TILs. As shown in FIG. 20, the statistical analysis of the frequencies of IL-21 secreting CD8⁺ TILs in different animals showed the significant increase of these cells in mice treated with a combination of anti-PD-L1 and E. coli^{pApyr} as compared to the groups treated with anti-PD-L1 alone or in combination with E. coli^{pHND19}.

### Example 10: Administration of E. coli^{pApyr} Results in Increase of IL-21 Secreting Cells Among CD8⁺ Cells Isolated from Ileal Peyer's Patches

Next, it was investigated whether E. coli^{pApyr} administration affected IL-21 secreting cells in the PPs of the small intestine, where T cell mediated immune responses are conditioned by the intestinal ecosystem.

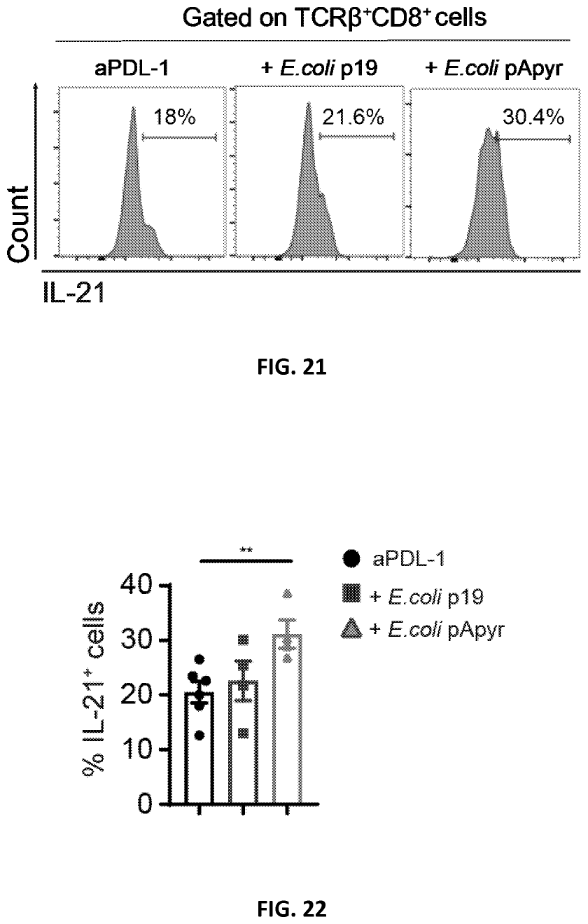

Results are shown in FIG. 21. Analogously to tumor tissue, IL-21 secreting cells were increased in PPs from mice treated with anti-PD-L1 together with E. coli^{pApyr} whereas the abundance of this cell population was similar in mice treated with anti-PD-L1 together with bacteria expressing the loss of function mutant of apyrase and mice receiving anti-PD-L1 without bacteria. This result shows that apyrase mediated conditioning of the microbiota results in induction of IL-21 secreting cells.

As shown in FIG. 22, statistical analysis of the frequencies of IL-21 secreting CD8⁺ cells in PPs from different animals showed the significant increase of these cells in mice treated with anti-PD-L1 and gavaged with E. coli^{pApyr} as compared to the groups treated with anti-PD-L1 alone or in combination with E. coli^{pHND19}.

### Example 11: Administration of E. coli^{pApyr} Results in Increase of Dendritic Cells Among CD3-Tumor Infiltrating Cells

The generation of effector T cells that can recognize and kill tumor cells requires professional antigen-presenting cells (APCs). Dendritic cells (DCs) are the most potent APCs and internalize, process and present tumor antigens to activate tumor-specific T cells. Upregulation of MHC-II contributes to generating a proficient tumoricidal T cell response. Therefore, DCs were identified by CD11c and MHCII among CD3⁻ cells infiltrating MC38 tumors in C57Bl/6 by flow cytometry as described in Example 6.

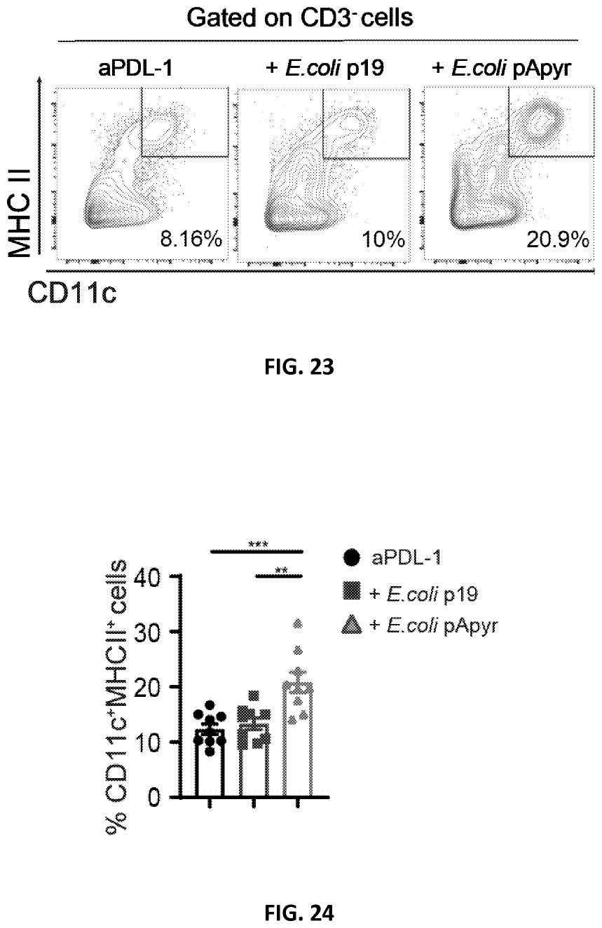

Results are shown in FIG. 23. The analysis of DCs identified by CD11c and MHCII among CD3⁻ cells infiltrating MC38 tumors in C57Bl/6 mice revealed the robust increase of DCs expressing high levels of MHCII when E. coli^{pApyr}, but not E. coli^{pHND19}, was combined with anti-PD-L1 antibodies, indicating that apyrase positively influenced DCs infiltration of tumors.

As shown in FIG. 24, statistical analysis of the frequencies of DCs infiltrating MC38 tumors in different animals showed the significant increase of these cells in mice treated with anti-PD-L1 and gavaged with E. coli^{pApyr} as compared to the groups treated with anti-PD-L1 alone or in combination with E. coli^{pHND19}.

The cDC1 migratory DCs subset, characterized by the expression of CD11c, MHC-II and CD103, induces cellular immunity against tumors. Therefore, this subset of DCs was further investigated by flow cytometry as described in Example 6.

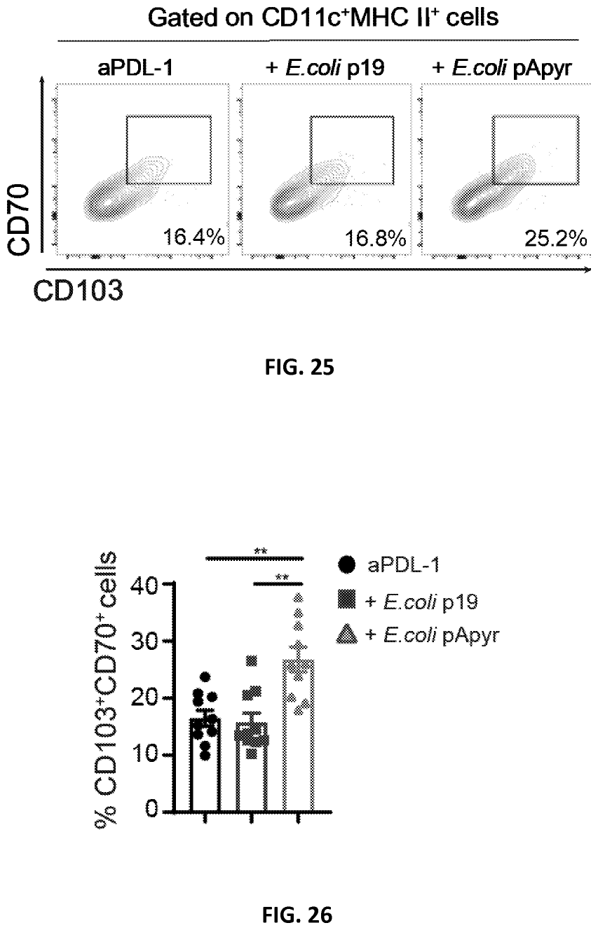

Results are shown in FIG. 25. Surprisingly, E. coli^{pApyr} enhanced the infiltration of CD103⁺CD70⁺ DCs into MC38 tumors when combined with anti-PD-L1 as compared to anti-PD-L1 alone or combined with E. coli^{pHND19}, thereby improving the efficacy of ICB therapy.

As shown in FIG. 26, the statistical analysis of the frequencies of CD103⁺CD70⁺ cells among CD11c⁺MHCII⁺ DCs infiltrating MC38 tumors in different animals showed the significant increase of these cells in mice treated with a combination of anti-PD-L1 and E. coli^{pApyr} as compared to the groups treated with anti-PD-L1 alone or in combination with E. coli^{pHND19}.

### Example 12: Enhancement of the Anti-Tumor Effect of Immune Checkpoint Inhibitors in an Experimental Model of CAR T Cell Therapy

Chimeric antigen receptor (CAR) T cell therapy targets adoptively transferred T cells directly to tumor cells to provide effective and durable anti-tumor responses (June, C. H., O'Connor, R. S., Kawalekar, O. U., Ghassemi, S., and Milone, M. C. (2018). CAR T cell immunotherapy for human cancer. Science 359, 1361-1365). The CAR endows transferred cells with high-avidity binding to cell-surface antigens independently from expression of major histocompatibility complex (MHC) and triggers robust T cell activation and anti-tumor response (Sadelain, M., Brentjens, R., and Rivière, I. (2013). The basic principles of chimeric antigen receptor design. Cancer Discov 3, 388-398). This therapeutic approach has been successfully applied to patients with chemotherapy-refractory hematologic malignancies (Park, J. H., Rivière, I., Gonen, M., Wang, X., Sénéchal, B., Curran, K. J., Sauter, C., Wang, Y., Santomasso, B., Mead, E., et al. (2018). Long-Term Follow-up of CD19 CAR Therapy in Acute Lymphoblastic Leukemia. N Engl J Med 378, 449-459; Schuster, S. J., Svoboda, J., Chong, E. A., Nasta, S. D., Mato, A. R., Anak, O., Brogdon, J. L., Pruteanu-Malinici, I., Bhoj, V., Landsburg, D., et al. (2017). Chimeric Antigen Receptor T Cells in Refractory B-Cell Lymphomas. N Engl J Med 377, 2545-2554). However, CAR T cell therapy could not be extended as effectively to solid tumors.

Factors that limit the success of CAR T cell therapy in solid tumors include the limited trafficking to the tumor and functional persistence of transferred cells due to immunosuppressive features of the TME. Therefore, combination with immune checkpoint inhibitors either extrinsically delivered or produced by CAR T cells themselves is assumed to antagonize these factors by fostering pro-inflammatory phenomena in the TME Grosser, R., Cherkassky, L., Chintala, N., and Adusumilli, P. S. (2019). Combination Immunotherapy with CAR T Cells and Checkpoint Blockade for the Treatment of Solid Tumors. Cancer Cell 36, 471-482). Preclinical studies have shown that combination of CAR T cell therapy with immune checkpoint inhibitors has increased efficacy over treatment with each agent alone, thereby supporting the translation of this approach to patients (Cherkassky, L., Morello, A., Villena-Vargas, J., Feng, Y., Dimitrov, D. S., Jones, D. R., Sadelain, M., and Adusumilli, P. S. (2016). Human CAR T cells with cell-intrinsic PD-1 checkpoint blockade resist tumor-mediated inhibition. J Clin Invest 126, 3130-3144; Hu, W., Zi, Z., Jin, Y., Li, G., Shao, K., Cai, Q., Ma, X., and Wei, F. (2019). CRISPR/Cas9-mediated PD-1 disruption enhances human mesothelin-targeted CAR T cell effector functions. Cancer Immunol Immunother 68, 365-377; John, L. B., Devaud, C., Duong, C. P., Yong, C. S., Beavis, P. A., Haynes, N. M., Chow, M. T., Smyth, M. J., Kershaw, M. H., and Darcy, P. K. (2013). Anti-PD-1 antibody therapy potently enhances the eradication of established tumors by gene-modified T cells. Clin Cancer Res 19, 5636-5646; Strome, S. E., Dong, H., Tamura, H., Voss, S. G., Flies, D. B., Tamada, K., Salomao, D., Cheville, J., Hirano, F., Lin, W., et al. (2003). B7-H1 blockade augments adoptive T-cell immunotherapy for squamous cell carcinoma. Cancer Res 63, 6501-6505).

In view thereof, it was investigated whether the combination of CAR T cell therapy with immune checkpoint inhibitors and (bacteria encoding) apyrase would further increase the anti-tumor effect. To this end, MC38 colon adenocarcinoma cells transfected with ovalbumin (MC38-OVA) and C57BL/6 mice were engrafted subcutaneously with 1×10⁶ OVA-expressing MC38 cells at day 0. At day 8, mice were injected intravenously with 8×10⁵ OT-I TCR transgenic T cells (congenially marked OT-I Rag1^{-/-}CD8⁺ cells, expressing a transgenic TCR specific for the H-2K^{b} restricted OVA peptide 257-264, isolated from spleen and lymph nodes of double mutant OT-I Rag1^{-/-} mice). Mice were injected intraperitoneally with anti-PD-L1 antibodies (100 μg/100 μl) at day 10, 14, 17 and 20, and gavaged every day from day 8 until the end of the experiment with 1×10¹⁰ of E. coli^{pApyr} or PBS. Tumor growth was scored with a caliper by measuring the greatest tumor diameter and its perpendicular to determine an average and then the area was calculated as: (average/2)²π.

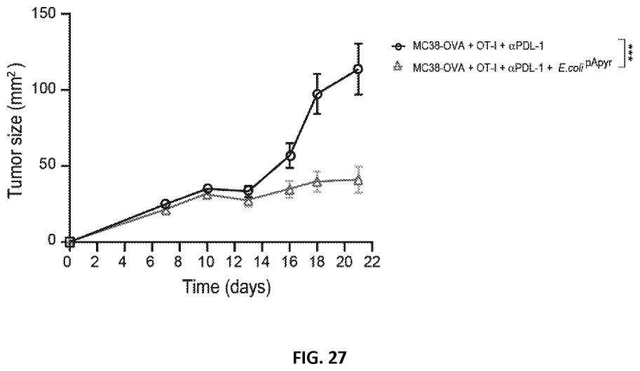

Results are shown in FIG. 27. Surprisingly, a significant reduction of tumor growth was observed in mice gavaged with E. coli^{pApyr} as compared to the group treated with PBS (in addition to OT-I TCR transgenic T cells and checkpoint inhibitor). Therefore, administration of (bacteria encoding) apyrase significantly enhanced the therapeutic effect of the checkpoint inhibitor in mice adoptively transferred with tumor specific cytotoxic T cells, an experimental model that reproduces therapeutic approaches with CAR T cells or infusion of in vitro expanded tumor infiltrating T cells.

### Example 13: Bacteria Expressing Apyrase Improve Anti-CTLA4 Treatment of Colon Adenocarcinoma

To investigate the effect of administration of bacteria expressing apyrase (obtained as described in Example 1) in combination with a different immune checkpoint inhibitor, colon adenocarcinoma MC38 cells were grafted subcutaneously into C57BL/6 mice.

The experiments were performed essentially as described in Example 2, with the difference that distinct tumor cells (MC38 colon adenocarcinoma cells) and a distinct immune checkpoint inhibitor (anti-CTLA4) were used. Briefly, colon adenocarcinoma MC38 cells were cultured in RPMI-1640 supplemented with 10% heat inactivate fetal bovine serum, 100 U/mL penicillin/streptomycin and 100 U/mL kanamycin. Cells were maintained in 5% CO₂ at 37° C. Tumor cells were harvested at exponential growth and subcutaneously engrafted in 8 week old C57Bl/6 mice at 1×10⁶ cells/100 ml (day 0).

Similarly as in Example 2, mice were orally gavaged with E. coli expressing apyrase (E. coli^{pApyr}) in combination with intra-peritoneal administration of anti-CTLA4. Mice were injected intraperitoneally with anti-CTLA4 monoclonal antibody (clone: 9H10; BioXCell) (100 μg/100 μl) at days 8, 11, 14, 18. E. coli^{pApyr} (1×10¹⁰ CFU) was administered daily by orogastric gavage from day 5 to termination of the experiment. Tumor growth was scored with a caliper by measuring the greatest tumor diameter and its perpendicular to determine an average and then the area was calculated as: (average/2)²□.

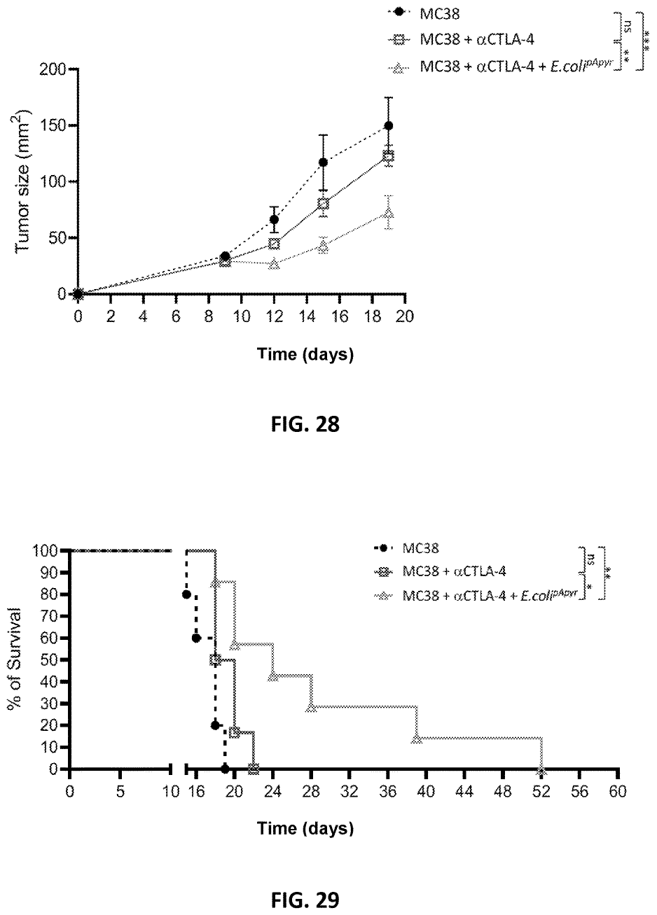

Results are shown in FIG. 28. Similarly as in Example 2, a significant reduction of tumor growth in mice treated with a combination of the checkpoint inhibitor (anti-CTLA4) and E. coli^{pApyr} was observed as compared to the group treated with anti-CTLA4 alone. Survival rates of the mice are shown in FIG. 29. Analysis of survival of mice following the engraftment of MC38 tumor revealed significantly enhanced survival in mice treated with a combination of anti-CTLA4 and E. coli^{pApyr} as compared to the group treated with anti-CTLA4 alone, thus confirming that administration of apyrase expressing bacteria improves the efficacy of the treatment with immune checkpoint inhibitors.

### Example 14: Bacteria Expressing Apyrase Improve Treatment of Colon Adenocarcinoma with the Combination of Anti-PD-L1 and Anti-CTLA4 Immune Checkpoint Inhibitors

To investigate the effect of administration of bacteria expressing apyrase together with a combination of two immune checkpoint inhibitors, colon adenocarcinoma MC38 cells were grafted subcutaneously into C57BL/6 mice.

The experiments were performed essentially as described in Example 2, with the difference that two distinct immune checkpoint inhibitors (anti-PD-L1 anti-CTLA4) were concomitantly administered and that MC38 colon adenocarcinoma cells were used. Briefly, colon adenocarcinoma MC38 cells were cultured in RPMI-1640 supplemented with 10% heat inactivate fetal bovine serum, 100 U/mL penicillin/streptomycin and 100 U/mL kanamycin. Cells were maintained in 5% CO₂ at 37° C. Tumor cells were harvested at exponential growth and subcutaneously engrafted in 8 week old C57Bl/6 mice at 1×10⁶ cells/100 ml (day 0).

Similarly as in Example 2, mice were orally gavaged with E. coli expressing apyrase (E. coli^{pApyr}) in combination with intra-peritoneal administration of anti-PD-L1 and anti-CTLA4. Mice were injected intraperitoneally with anti-PD-L1 (clone: 10F.9G2; BioXCell) and anti-CTLA4 (clone: 9H10; BioXCell) monoclonal antibodies (100 μg/100 μl of each) at day 8, 11, 14, 18. E. coli^{pApyr} (1×10¹⁰ CFU) was administered daily by orogastric gavage from day 5 to termination of the experiment. Tumor growth was scored with a caliper by measuring the greatest tumor diameter and its perpendicular to determine an average and then the area was calculated as: (average/2)²□.

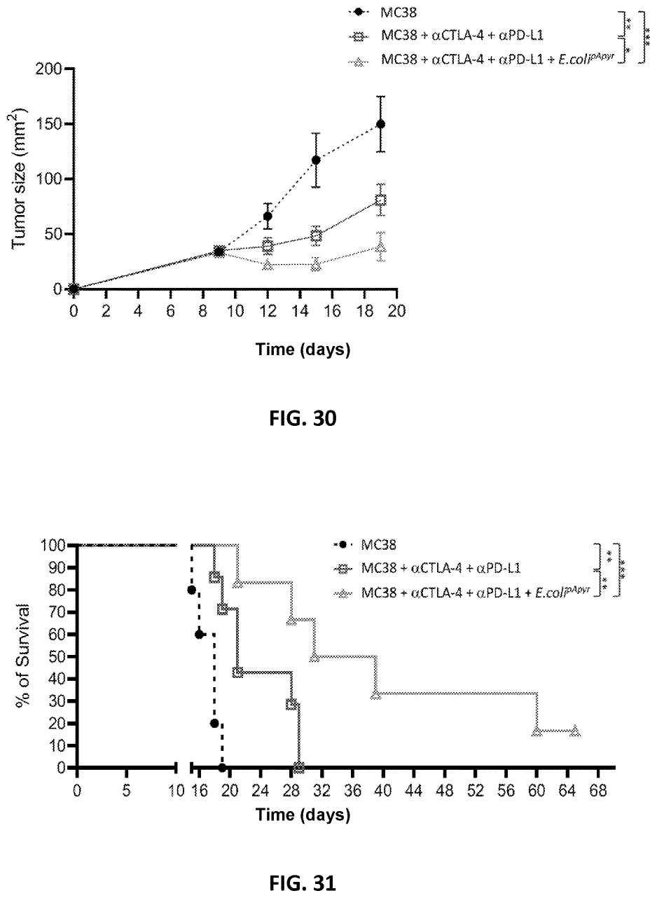

Results are shown in FIG. 30. Similarly as in Example 2, a significant reduction of tumor growth in mice treated with the combination of anti-PD-L1 and anti-CTLA4 together with E. coli^{pApyr} was observed as compared to the group treated with anti-PD-L1 and anti-CTLA4 without bacteria. Survival rates of the mice are shown in FIG. 31. Analysis of survival of mice following the engraftment of MC38 tumor revealed significantly enhanced survival in mice treated with the combination of anti-PD-L1 and anti-CTLA4 together with E. coli^{pApyr} as compared to the group treated with antibodies without bacteria, thus confirming that administration of apyrase expressing bacteria improves the efficacy of the treatment with immune checkpoint inhibitors.

### Example 15: Bacteria Expressing Apyrase Improve Anti-PD-L1 Treatment of Colon Adenocarcinoma in Balb/c Mice

To investigate the effect of administration of bacteria expressing apyrase in combination with an immune checkpoint inhibitor on a tumor model in a distinct mouse strain, colon adenocarcinoma CT26 cells were grafted subcutaneously into Balb/c mice.

The experiments were performed essentially as described in Example 2, with the difference that a distinct mouse strain and syngeneic tumor cells were used. Briefly, colon adenocarcinoma CT26 cells were cultured in RPMI-1640 supplemented with 10% heat inactivate fetal bovine serum, 100 U/mL penicillin/streptomycin and 100 U/mL kanamycin. Cells were maintained in 5% CO₂ at 37° C. Tumor cells were harvested at exponential growth and subcutaneously engrafted in 8 week old Balb/c mice at 1×10⁶ cells/100 ml (day 0).

Similarly as in Example 2, mice were orally gavaged with E. coli expressing apyrase (E. coli^{pApyr}) or transformants with empty vector (E. coli^{pBAD28}) in combination with intra-peritoneal administration of anti-PD-L1. Mice were injected intraperitoneally with anti-PD-L1 monoclonal antibody (clone: 10F.9G2; BioXCell) (100 μg/100 μl) at day 8, 11, 14, 18. E. coli^{pApyr} or E. coli^{pBAD28} (1×10¹⁰ CFU) was administered daily by orogastric gavage from day 5 to termination of the experiment. Tumor growth was scored with a caliper by measuring the greatest tumor diameter and its perpendicular to determine an average and then the area was calculated as: (average/2)²□.

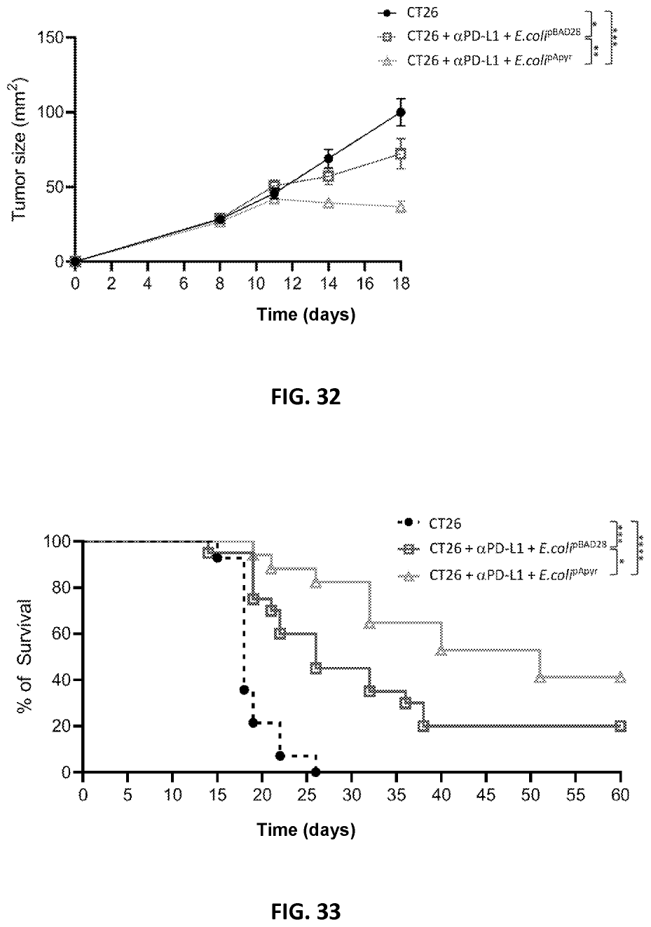

Results are shown in FIG. 32. Similarly as in Example 2, a significant reduction of tumor growth in mice treated with a combination of anti-PD-L1 and E. coli^{pApyr} was observed as compared to the groups treated with anti-PD-L1 alone or in combination with E. coli^{pBAD28} Survival rates of the mice are shown in FIG. 33. Analysis of survival of mice following the engraftment of CT26 tumor revealed significantly enhanced survival in mice treated with a combination of anti-PD-L1 and E. coli^{pApyr} as compared to the groups treated with anti-PD-L1 in combination with E. coli^{pBAD28}, thus confirming that administration of apyrase expressing bacteria improves the efficacy of the treatment with immune checkpoint inhibitors.

### Example 16: Administration of E. coli^{pApyr} Results in Increase of CCR9⁺ Cells Among CD8⁺ TILs

The migratory phenotype of T cells contributes to immunosurveillance against tumors. High frequencies of CD8⁺CCR9⁺ cells correlated with prolonged overall survival in melanoma patients and mice with a spontaneous melanoma. Accordingly, neutralization of the exclusive CCR9 ligand, the chemokine CCL25, accelerated tumor outgrowth (Jacquelot, N., Enot, D. P., Flament, C., Vimond, N., Blattner, C., Pitt, J. M., Yamazaki, T., Roberti, M. P., Daillère, R., Vétizou, M., et al. 2016. Chemokine receptor patterns in lymphocytes mirror metastatic spreading in melanoma. The Journal of clinical investigation, 126, 921). CD8⁺CCR9⁺ T cells display enhanced activation and their recruitment by intratumoral delivery of CCL25 induced anti-tumor immunity (Chen, H., Cong, X., Wu, C., Wu, X., Wang, J., Mao, K., Li, J., Zhu, G., Liu, F., Meng, X., et al. 2020. Intratumoral delivery of CCL25 enhances immunotherapy against triple-negative breast cancer by recruiting CCR9⁺ T cells. Science Advances 6, eaax4690).

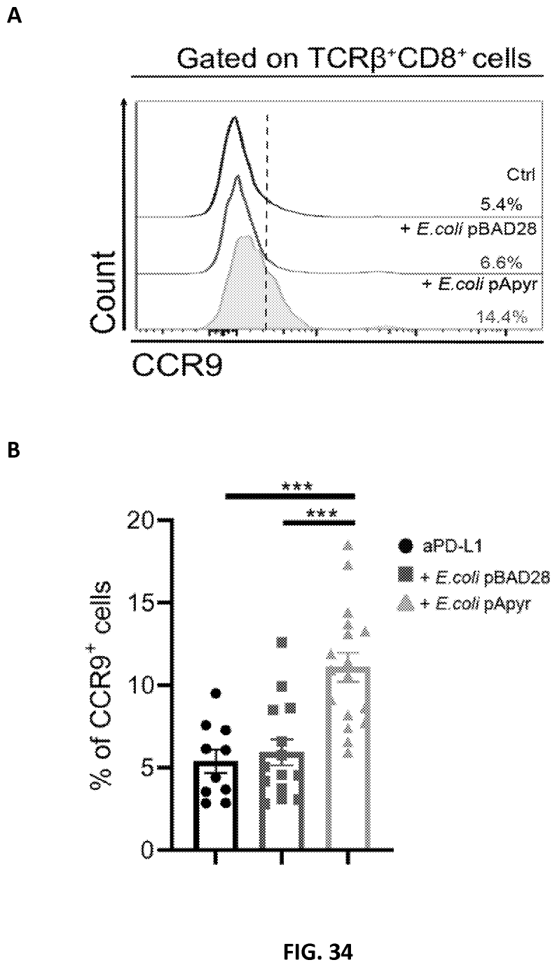

In view thereof, CCR9 expression was analyzed by flow cytometry on electronically gated CD8⁺ TILs as described above in Example 6. Results are shown in FIG. 34. Strikingly, TILs isolated from MC38 tumors resected from mice treated with a combination of anti-PD-L1 and E. coli^{pApyr} showed an increase of CCR9⁺ cells among electronically gated CD8⁺ TILs as compared to the mice treated with anti-PD-L1 alone or in combination with E. coli^{pBAD28}. As shown in FIG. 34, statistical analysis of the frequencies of CCR9⁺CD8⁺ TILs in tumors from different animals showed the significant increase of these cells in mice treated with a combination of anti-PD-L1 and E. coli^{pApyr} as compared to the groups treated with anti-PD-L1 alone or in combination with E. coli^{pBAD28}.

### Example 17: Administration of E. coli^{pApyr} Results in Increase of Ki-67⁺ Cells Among CD8⁺ T Cells in Peyer's Patches of the Ileum

CD8⁺CCR9⁺ cells are generated in the gut associated lymphoid tissue (GALT) and preferentially home to the small intestinal epithelium.

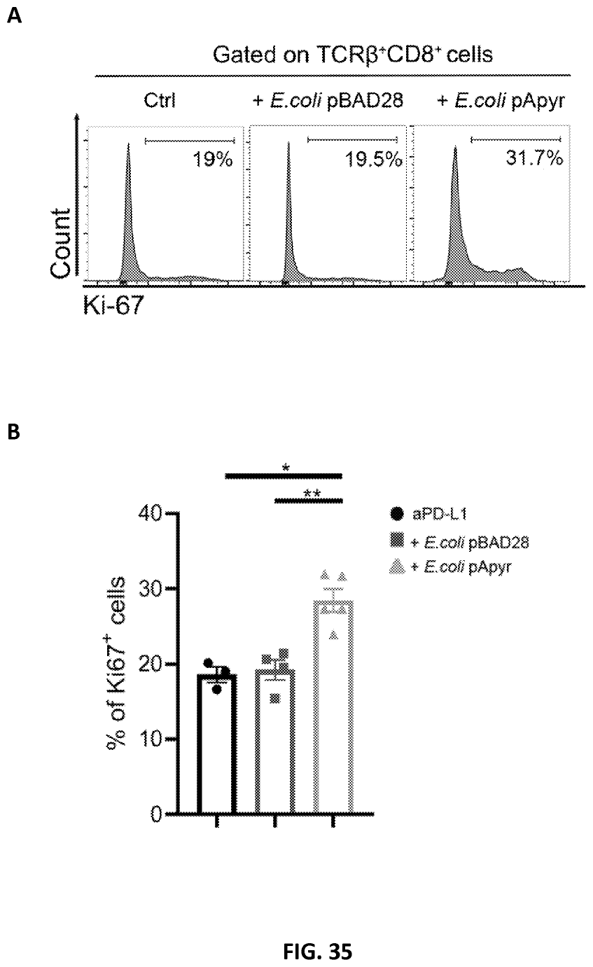

To address whether E. coli^{pApyr} administration could affect CD8⁺ cells expansion in the Peyer's patches (PPs) of the small intestine, their proliferative activity was investigated. Electronically gated CD8⁺ cells stained for the nuclear protein Ki-67, that is strictly associated to cell proliferation, was analyzed by flow cytometry. Results are shown in FIG. 35. Strikingly, CD8⁺ cells isolated from PPs harvested from mice treated with a combination of anti-PD-L1 and E. coli^{pApyr} showed an increase of Ki-67⁺ cells among electronically gated CD8⁺ T cells as compared to mice treated with anti-PD-L1 alone or in combination with E. coli^{pBAD28}. As shown in FIG. 35, statistical analysis of the frequencies of Ki-67⁺CD8⁺ T cells in PPs from different animals showed the significant increase of these cells in mice treated with a combination of anti-PD-L1 and E. coli^{pApyr} as compared to the groups treated with anti-PD-L1 alone or in combination with E. coli^{pBAD28}.

### Example 18: Administration of E. coli^{pApyr} Results in Increase of T-Bet⁺ Cells Among CD8⁺ T Cells in Peyer's Patches of the Ileum

The generation and function of effector CD8⁺ T cells relies on the T-box transcription factor T-bet (Tbx21) (Sullivan, B. M., Juedes, A., Szabo, S. J., von Herrath, M., and Glimcher, L. H. 2003. Antigen-driven effector CD8 T cell function regulated by T-bet. Proceedings of the National Academy of Sciences 100, 15818). An effective anti-tumor response during checkpoint blockade treatment depends on T-bet induction that is required for IFN-γ production and TILs cytotoxicity (Berrien-Elliott, M. M., Yuan, J., Swier, L. E., Jackson, S. R., Chen, C. L., Donlin, M. J., and Teague, R. M. 2015. Checkpoint Blockade Immunotherapy Relies on T-bet but Not Eomes to Induce Effector Function in Tumor-Infiltrating CD8⁺ T Cells. Cancer Immunology Research 3, 116).

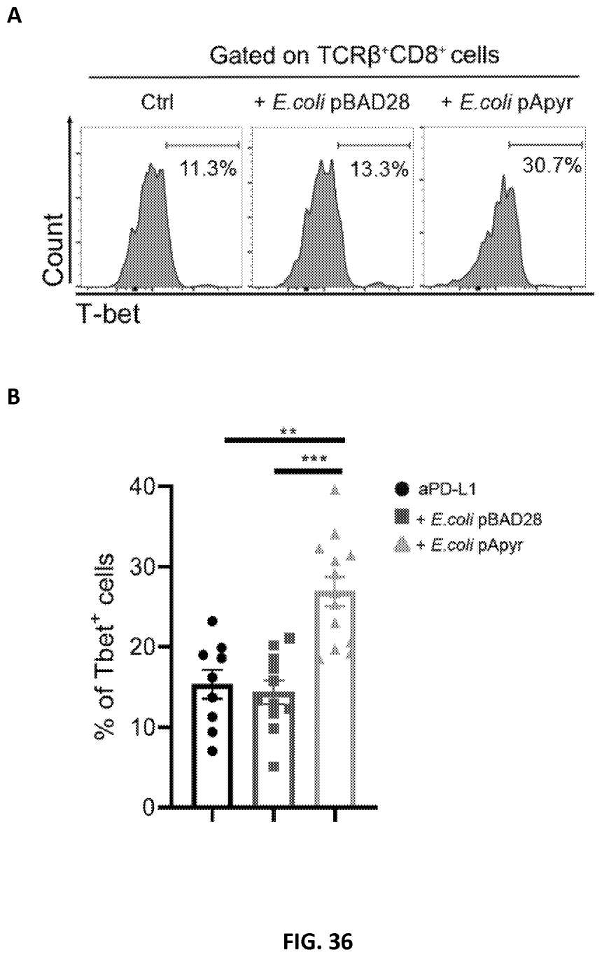

In view thereof, it was investigated whether E. coli^{pApyr} administration affected T-bet expression in CD8⁺ cells in the PPs of the small intestine. Electronically gated CD8⁺ cells stained for T-bet were analyzed by flow cytometry. Results are shown in FIG. 36. Strikingly, CD8⁺ cells isolated from PPs harvested from mice treated with a combination of anti-PD-L1 and E. coli^{pApyr} showed an increase of T-bet⁺ cells among electronically gated CD8⁺ T cells as compared to the mice treated with anti-PD-L1 alone or in combination with E. coli^{pBAD28}. As shown in FIG. 36, statistical analysis of the frequencies of T-bet⁺CD8⁺ T cells in PPs from different animals showed the significant increase of these cells in mice treated with a combination of anti-PD-L1 and E. coli^{pApyr} as compared to the groups treated with anti-PD-L1 alone or in combination with E. coli^{pBAD28}.

### Example 19: Design and Production of Apyrase Expressing Lactococcus lactis

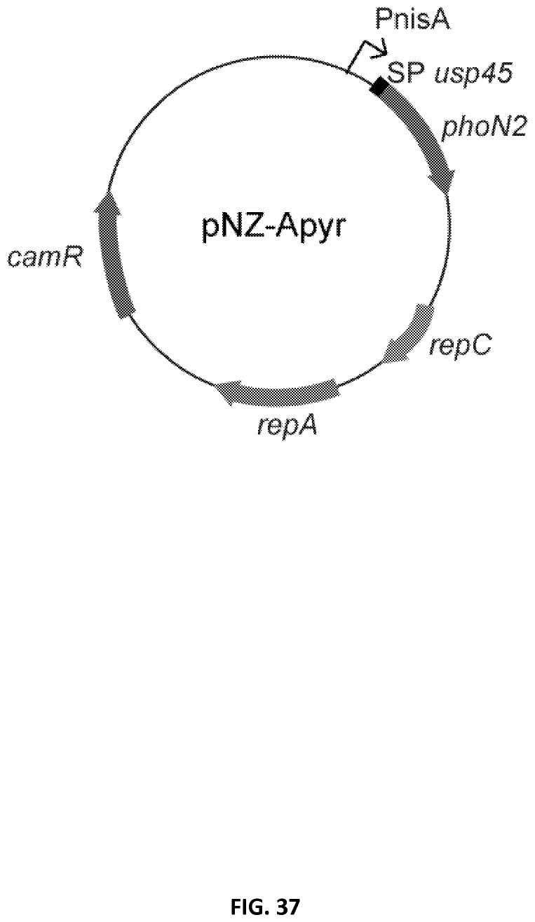

For the expression of Shigella flexneri apyrase in the Lactococcus lactis NZ900 strain, the apyrase encoding gene phoN2 was PCR amplified from the S. flexneri genome and cloned into the pNZ8123 plasmid, generating the pNZ-Apyr plasmid (FIG. 37). Apyrase expression in the pNZ-Apyr plasmid is controlled by the PnisA promoter, which is inducible by the nisin anti-microbial peptide. The phoN2 gene was in-frame cloned with the signal sequence of the L. lactis major secreted protein Usp45 to allow apyrase secretion. L. lactis^{pNZ} and L. lactis^{pNZ-Apyr} strains were grown in M17 medium supplemented with glucose (0.5% w/v) and nisin (4 ng/ml).

### Example 20: Probiotic Bacteria of the Order Lactobacillales Expressing Apyrase Improve Anti-PD-L1 Treatment of Colon Adenocarcinoma

To investigate the effect of administration of distinct probiotic bacteria delivering apyrase to the ileum in combination with an immune checkpoint inhibitor, colon adenocarcinoma MC38 cells were grafted subcutaneously into C57BL/6 mice that were subsequently gavaged with the Lactobacillus strain Lactococcus lactis either expressing or not apyrase.

The experiments were performed essentially as described in Example 2, with the difference that Lactococcus lactis as described in Example 19 above was used. Briefly, colon adenocarcinoma MC38 cells were cultured in RPMI-1640 supplemented with 10% heat inactivate fetal bovine serum, 100 U/mL penicillin/streptomycin and 100 U/mL kanamycin. Cells were maintained in 5% CO₂ at 37° C. Tumor cells were harvested at exponential growth and subcutaneously engrafted in 8 week old C57Bl/6 mice at 1×10⁶ cells/100 ml (day 0). L. lactis transformant with empty vector (L. lactis^{pNZ}) or L. lactis expressing apyrase (L. lactis^{pNZ-Apyr}) were grown in M17 medium supplemented with chloramphenicol (10 μg/ml), glucose (0.5% w/v) and nisin (4 ng/ml).

Similarly as in Example 2, mice were orally gavaged with L. lactis^{pNZ} or L. lactis^{pNZ-Apyr} in combination with intra-peritoneal administration of anti-PD-L1. Mice were injected intraperitoneally with anti-PD-L1 monoclonal antibody (clone: 10F.9G2; BioXCell) (100 μg/100 μl) at day 8, 11, 14, 17. L. lactis^{pNZ} or L. lactis^{pNZ-Apyr} (1×10¹⁰ CFU) were administered daily by orogastric gavage from day 5 to termination of the experiment. Tumor growth was scored with a caliper by measuring the greatest tumor diameter and its perpendicular to determine an average and then the area was calculated as: (average/2)²□.

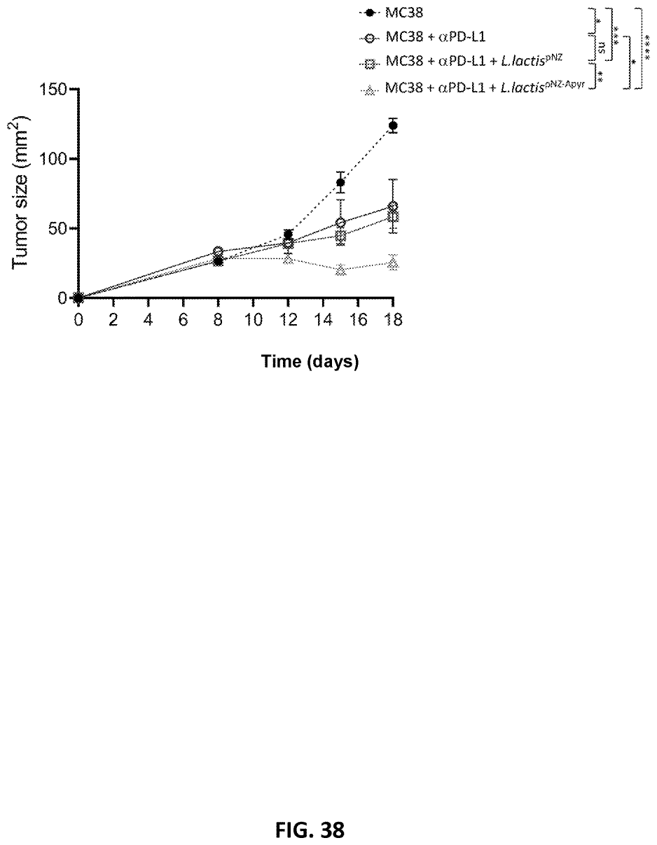

Results are shown in FIG. 38. Similarly as in Example 2, a significant reduction of tumor growth in mice treated with a combination of anti-PD-L1 and L. lactis^{pNZ-Apyr} was observed as compared to the groups treated with anti-PD-L1 in combination with L. lactis^{pNZ}.

### Example 21: Generation of Recombinant Bacteria Heterologously Expressing Apyrase, which Carry the Apyrase Gene Integrated in their Genome (EcN::Phon2)

The apyrase expressing bacteria designed and produced as described in Examples 1 and 19 above were obtained by transforming bacteria with plasmids encoding apyrase. Such plasmids may contain antibacterial resistance for the selection of the transformants. Such bacterial transformants typically bear multiple copies of the apyrase-encoding plasmid (and may be selected for antibiotic resistance). To investigate whether similar effects can be obtained in recombinant bacteria encoding apyrase in a heterologous manner in a single copy in their genome instead of multiple copies of extrachromosomal plasmids, bacteria having a single copy of the (heterologous) apyrase (phoN2) gene in the bacterial chromosome (non-transmissible) (without antibiotic resistance) were created.

To this end, the chromosomal integration of the Shigella flexneri phoN2 apyrase-encoding gene in the EcN genome (GenBank accession number CP007799.1) was performed by the λ Red recombineering approach (Datsenko K. A. and Wanner B. L. 2000 One-step inactivation of chromosomal genes in Escherichia coli K-12 using PCR products. Proc Natl Acad Sci USA. 97, 6640).

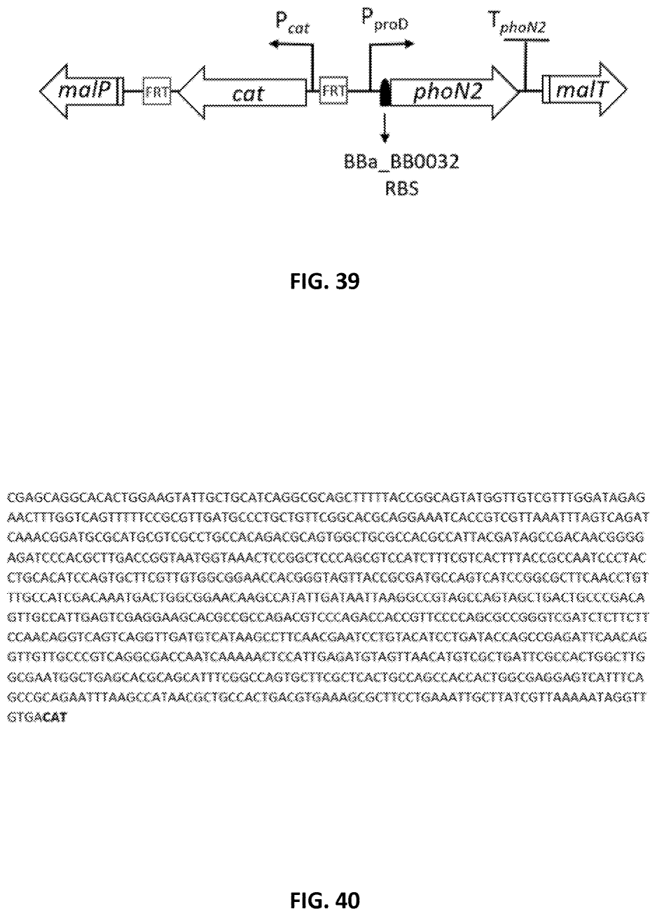

FIG. 39 schematically shows the DNA fragment used for the recombineering, including:
A portion of the EcN malP gene, coding for the maltodextrin phosphorylase enzyme;
The E. coli cat gene, which codes for the chloramphenicol acetyltransferase enzyme conferring resistance to the chloramphenicol antibiotic, flanked by the Flippase Recognition Target (FRT) sequences;
The Shigella flexneri phoN2 apyrase-encoding gene fused upstream with the P_{proD} synthetic promoter (Davis J. H., Rubin A. J. and Sauer R. T. 2011 Design, construction and characterization of a set of insulated bacterial promoters. Nucleic Acids Res. 9, 1131) and the BBa_BB0032 Ribosome Binding Site (RBS; iGEM Parts Registry), and downstream with the phoN2 transcriptional terminator;
A portion of the EcN malT gene, coding for the transcriptional activator of the maltose and maltodextrins operon.

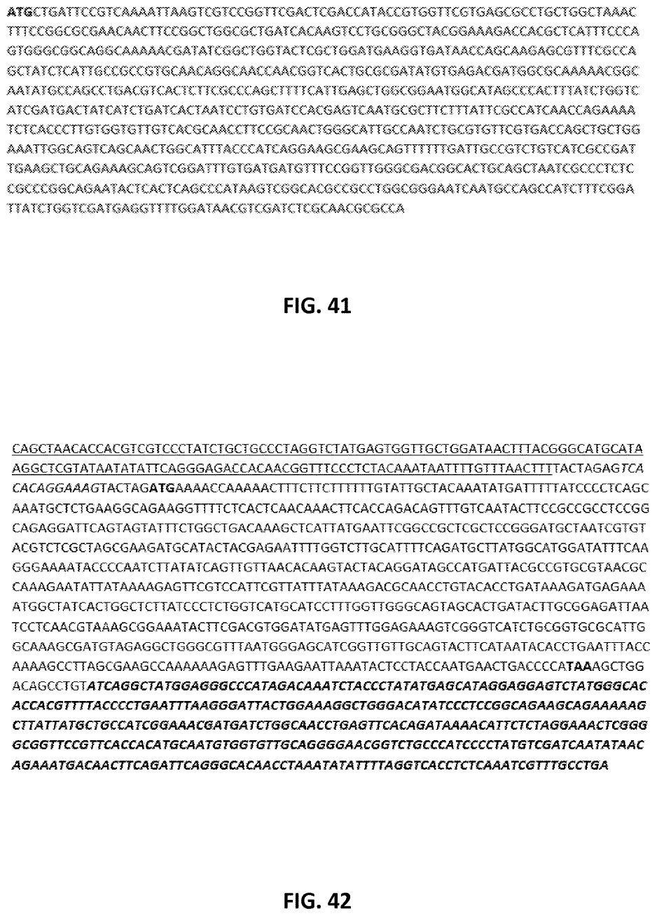

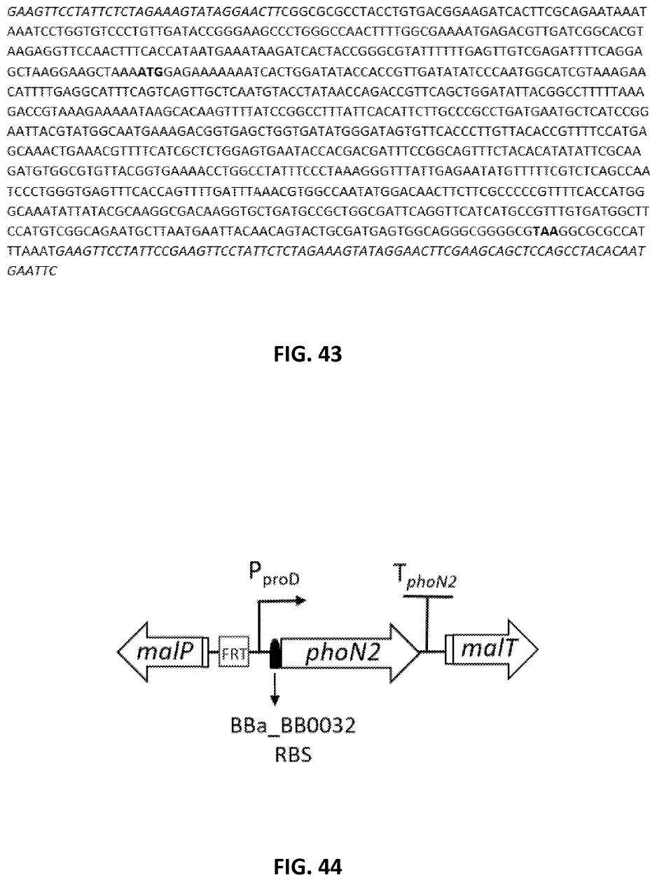

FIGS. 40 and 41 show the nucleotide sequences of EcN malP and malT gene portions, respectively (SEQ ID NOs 4 and 5, respectively). FIG. 42 shows the nucleotide sequence of the DNA fragment, including the P_{proD} promoter, the BBa_BB0032 RBS, the S. flexneri phoN2 gene and the phoN2 transcriptional terminator (SEQ ID NO: 6). FIG. 43 shows the nucleotide sequence of the DNA fragment, including the E. coli cat gene flanked by the FRT sequences (SEQ ID NO: 7).

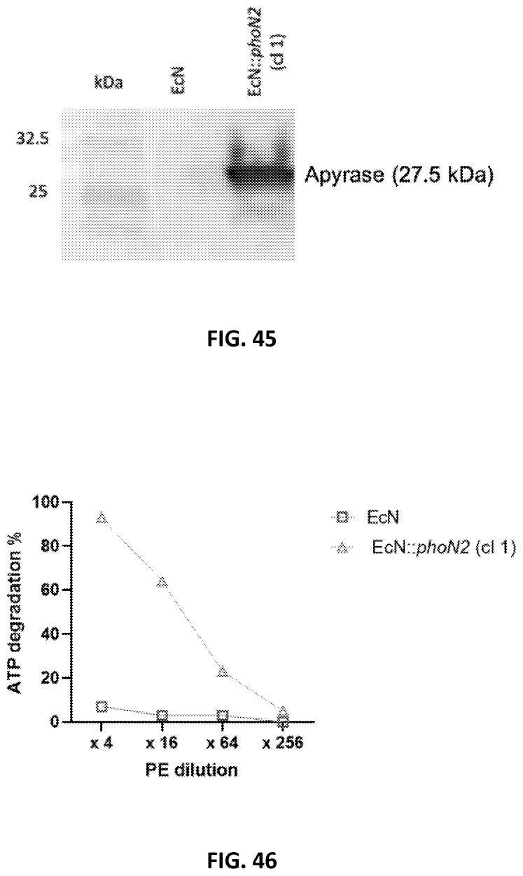

To perform the recombineering in the EcN genome, the insertion DNA fragment was transformed in an EcN strain carrying the pKD46 plasmid, which expresses the phage \ Red recombinase. The \ Red-mediated homology recombination at the malP and malT sites promoted the integration of the insertion DNA fragment in the malP-malT intergenic region of EcN. After pKD46 removal, the EcN clones carrying the insertion DNA fragment in the genome were selected for chloramphenicol resistance and checked by PCR for the correct integration in the genome. The EcN clones selected for the correct integration of the insertion DNA fragment were transformed with the pCP20 plasmid, which expresses the yeast Flp recombinase (Flippase), to excise the chloramphenicol resistance cassette from the genome. After pCP20 removal, the EcN recombinant clones not carrying the chloramphenicol cassette in the genome were selected for chloramphenicol sensitivity and checked by PCR for the correct excision of the cassette from the genome. The resulting EcN recombinant clones carrying the S. flexneri phoN2 gene in the malP-malT intergenic region were named EcN::phoN2. FIG. 44 schematically shows the malP-phoN2-malT recombinant genomic region of the obtained EcN::phoN2 clones. FIG. 45 shows the expression of apyrase in one selected EcN::phoN2 clone (cl 1) in a Western-Blot of periplasmic extracts. In addition, the activity of the enzyme in EcN::phoN2 cl 1 was verified. FIG. 46 shows the dose-dependent degradation of ATP by EcN::phoN2 cl 1 periplasmic extract in an in vitro ATP-degradation assay. In both assays, the EcN wild type strain (EcN) was used as negative control. The EcN wild type and EcN::phoN2 bacterial strains were grown in LB medium.

### Example 22: Recombinant Bacteria Encoding Apyrase in their Genome for Heterologous Expression Improve Anti-PD-L1 Treatment of Colon Adenocarcinoma

To investigate whether administration of E. coli Nissle 1917 (EcN) probiotic bacteria with phoN2 gene integrated in the genome (obtained as described above, Example 21) were effective in enhancing the control of tumor growth induced by an immune checkpoint inhibitor, colon adenocarcinoma MC38 cells were grafted subcutaneously into C57BL/6 mice that were subsequently gavaged with Ecn or EcN::phoN2 strain.

The experiments were performed essentially as described in Example 2, with the difference that the bacteria as described above (Example 21: EcN and EcN::phoN2) were used. Briefly, colon adenocarcinoma MC38 cells were cultured in RPMI-1640 supplemented with 10% heat inactivate fetal bovine serum, 100 U/mL penicillin/streptomycin and 100 U/mL kanamycin. Cells were maintained in 5% CO₂ at 37° C. Tumor cells were harvested at exponential growth and subcutaneously engrafted in 8 week old C57Bl/6 mice at 1×10⁶ cells/100 ml (day 0). EcN and EcN::phoN2 were grown in LB medium.

Similarly as in Example 2, mice were orally gavaged with Ecn or EcN::phoN2 in combination with intra-peritoneal administration of anti-PD-L1. Mice were injected intraperitoneally with anti-PD-L1 monoclonal antibody (clone: 10F.9G2; BioXCell) (100 μg/100 μl) at day 8, 11, 14, 17. Ecn or EcN::phoN2 (1×10¹⁰ CFU) were administered daily by orogastric gavage from day 5 to termination of the experiment. Tumor growth was scored with a caliper by measuring the greatest tumor diameter and its perpendicular to determine an average and then the area was calculated as: (average/2)²□.

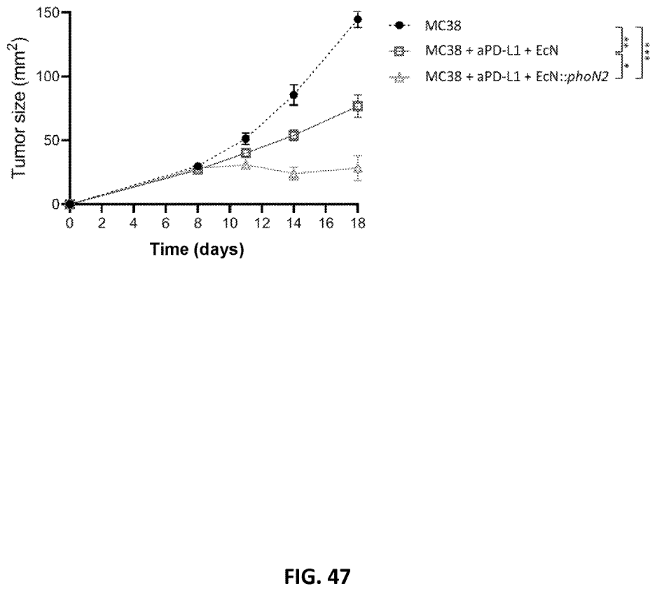

Results are shown in FIG. 47. Similarly as in Example 2, a significant reduction of tumor growth in mice treated with a combination of anti-PD-L1 and EcN::phoN2 strain was observed as compared to the group treated with anti-PD-L1 in combination with EcN.

### Example 23: Generation of a Salmonella enterica Serovar Typhimurium Strain Expressing a Tumor Antigen (Chicken Ovalbumin) and Apyrase

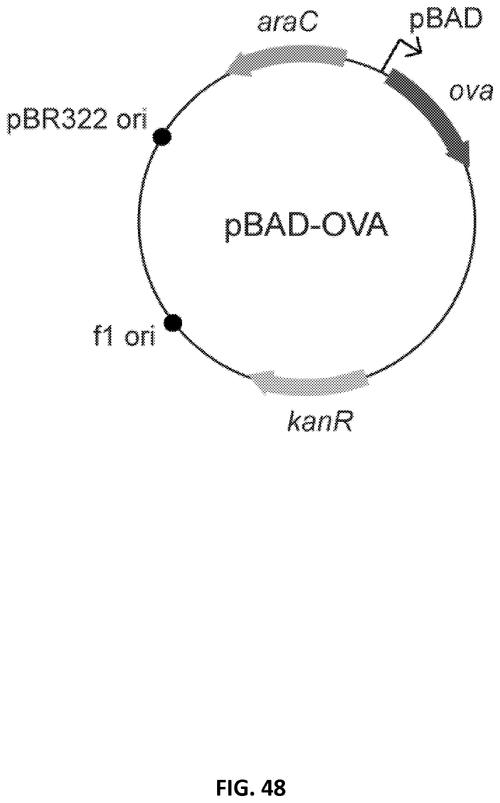

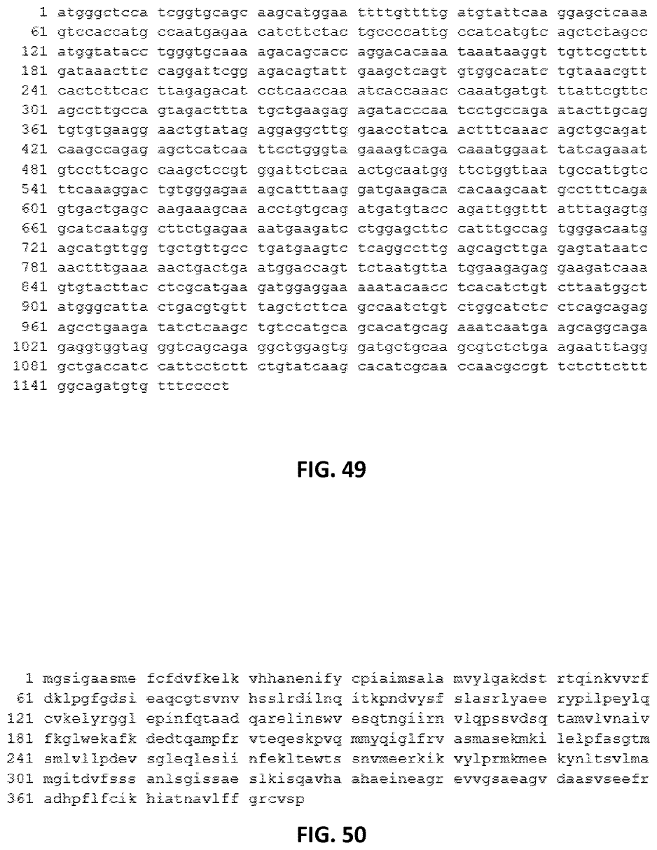

For the expression of chicken ovalbumin in the attenuated Salmonella enterica serovar Typhimurium AaroA (S. Tm) strain, the cDNA encoding ovalbumin (ova) was PCR amplified from the pcDNA3 plasmid and cloned into the pBAD18-Kan plasmid, generating the pBAD-OVA plasmid (FIG. 48). The arabinose-inducible pBAD promoter controls ovalbumin expression in S. Tm^{pBAD-OVA} strain. The ovalbumin cDNA and protein sequences are depicted in FIGS. 49 and 50, respectively (SEQ ID NOs 8 and 9, respectively).

S. Tm^{pBAD-OVA} strain was then transformed with the pHND10 plasmid in order to generate the S. Tm^{pApyr-OVA} strain expressing both chicken ovalbumin and Shigella flexneri apyrase. S. Tm^{pBAD-OVA} strain was grown in LB medium supplemented with kanamycin (25 μg/ml) and arabinose (0.1% w/v). S. Tm^{pApyr-OVA} strain was grown in LB medium supplemented with ampicillin (100 μg/ml), chloramphenicol (30 μg/ml), kanamycin (25 μg/ml) and arabinose (0.1% w/v).

### Example 24: Immunization with Bacteria Expressing Apyrase and a Tumor Antigen (OVA) Results in Rejection of Colon Adenocarcinoma by Anti-PD-L1 Treatment

To address whether combined expression of apyrase and a tumor antigen (OVA) could amplify rejection of colon adenocarcinoma induced by oral immunization with S. Tm expressing OVA as a tumor antigen, colon adenocarcinoma MC38-OVA cells were grafted subcutaneously into C57BL/6 mice that were subsequently immunized with S. Tm^{pApyr-OVA} or S. Tm^{pBAD-OVA} by orogastric gavage.

Colon adenocarcinoma MC38-OVA cells were cultured in RPMI-1640 supplemented with 10% heat inactivate fetal bovine serum, 100 U/mL penicillin/streptomycin and 100 U/mL kanamycin. Cells were maintained in 5% CO₂ at 37° C. Tumor cells were harvested at exponential growth and subcutaneously engrafted in 8 week old C57Bl/6 mice at 1×10⁶ cells/100 ml (day 0). S. Tm^{pBAD-OVA} strain was grown in LB medium supplemented with kanamycin (25 μg/ml) and arabinose (0.1% w/v). S. Tm^{pApyr-OVA} strain was grown in LB medium supplemented with ampicillin (100 μg/ml), chloramphenicol (30 μg/ml), kanamycin (25 μg/ml) and arabinose (0.1% w/v). Mice were immunized by oral gavage with 1×10⁹ S. Tm^{pBAD-OVA} or S. Tm^{pApyr-OVA} at day 5 and 10 after tumor engraftment. On day 8, 11 and 14 after tumor inoculation, mice were treated i.p. with 100 μg of anti-PD-L1 antibody in 100 μl PBS i.p. The presence of the tumor was established at day 17.

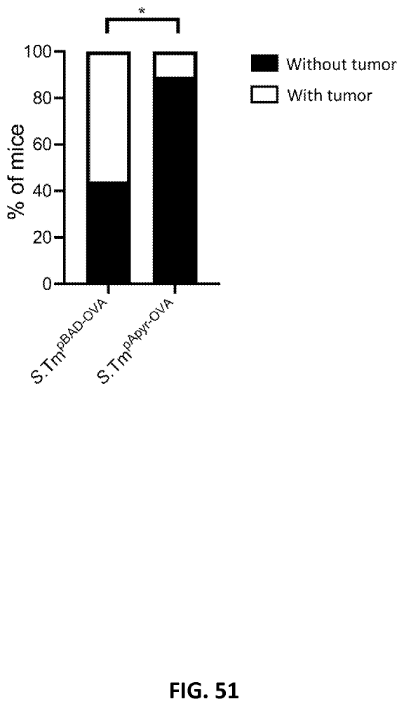

Results are shown in FIG. 51. A significant increase in the percentage of animals showing no sign of tumor was observed in the group of mice treated with anti-PD-L1 and immunized with bacteria expressing both, apyrase and the tumor antigen (S. Tm^{pApyr-OVA}) as compared to the group treated with anti-PD-L1 and immunized with the bacteria expressing the tumor antigen only (S. Tm^{pBAD-OVA}).

### Example 25: Bacteria Expressing Apyrase Improve Treatment of Colon Adenocarcinoma by Inducing Tumor Infiltration by Newly Generated T Cells

As mentioned above, CD8⁺CCR9⁺ cells are generated in the gut associated lymphoid tissue (GALT) and preferentially home to the small intestinal epithelium. The increase of CD8⁺CCR9⁺ cells in the tumor microenvironment of mice treated with a combination of anti-PD-L1 and E. coli^{pApyr}, but not E. coli^{pBAD28} (Example 16; FIG. 34) suggests that apyrase might promote the generation of these cells with tumoricidal function in the GALT, e.g. the Peyer's patches (PP). Fingolimod (FTY720) is a functional antagonist of the S1P1 receptor that blocks the egress of T cells from lymphoid organs (Matloubian, M., Lo, C. G., Cinamon, G., Lesneski, M. J., Xu, Y., Brinkmann, V., Allende, M. L., Proia, R. L., and Cyster, J. G. 2004. Lymphocyte egress from thymus and peripheral lymphoid organs is dependent on S1P receptor 1. Nature 427, 355). Therefore, to address whether apyrase was promoting in the PPs the generation of CD8⁺CCR9⁺ cells that could migrate to the tumor and control tumor growth, mice were treated with FTY720 the day before initiating the treatment with anti-PD-L1 antibody to block the egress of T cells from the PPs.

The experiments were performed essentially as described in Example 3, with the difference that FTY720 was administered i.p. from day 7 after tumor engraftment every 2 days until the end of the experiments. Briefly, colon adenocarcinoma MC38 cells were cultured in RPMI-1640 supplemented with 10% heat inactivate fetal bovine serum, 100 U/mL penicillin/streptomycin and 100 U/mL kanamycin. Cells were maintained in 5% CO₂ at 37° C. Tumor cells were harvested at exponential growth and subcutaneously engrafted in 8 week old C57Bl/6 mice at 1×10⁶ cells/100 DI (day 0).

Similarly as in Example 3, mice were orally gavaged with E. coli expressing apyrase (E. coli^{pApyr}) in combination with intra-peritoneal administration of anti-PD-L1 either preceded or not by i.p. injection of 1 mg/kg of FTY720 at day 7 and every 2 days thereafter. Mice were injected intraperitoneally with anti-PD-L1 (clone: 10F.9G2; BioXCell) monoclonal antibody (100 μg/100 μl of each) at day 8, 11, 14, 17. E. coli^{pApyr} (1×10¹⁰ CFU) was administered daily by orogastric gavage from day 5 to termination of the experiment. Tumor growth was scored with a caliper by measuring the greatest tumor diameter and its perpendicular to determine an average and then the area was calculated as: (average/2)²□.

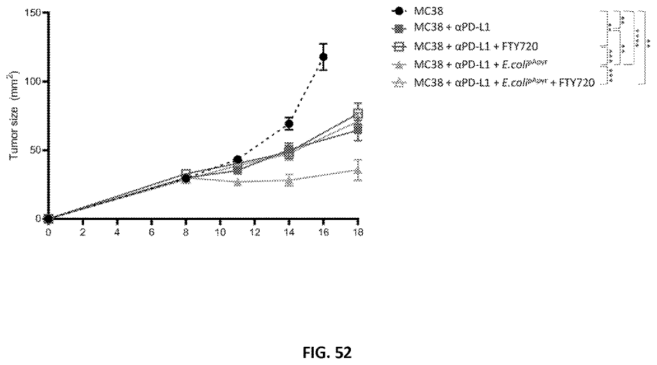

Results are shown in FIG. 52. Similarly as in Example 3, a significant reduction of tumor growth in mice treated with the combination of anti-PD-L1 and E. coli^{pApyr} was observed as compared to the group treated with stand-alone anti-PD-L1. However, the beneficial effect of E. coli^{pApyr} on tumor growth was lost in the group of mice that were treated also with FTY720. As previously shown (Chow M. T., Ozga A. J., Servis R. L., Frederick D. T., Lo J. A., Fisher D. E., et al. 2019 Intratumoral activity of the CXCR3 chemokine system is required for the efficacy of anti-PD-1 therapy. Immunity 50, 1498.), tumor control induced by anti-PD-L1 was not influenced by FTY720 because it is predominantly mediated by T cells already present in the tumor microenvironment before the initiation of the anti-PD-L1 treatment and does not depend on newly generated cytotoxic T cells.

### Example 26: Blockade of T Cells Egress from Lymphoid Organs Inhibits the Increase of CCR9⁺ and ICOS⁺ Cells Among CD8⁺ TILs Mediated by E. coli^{pApyr}

To address whether the increase of CD8⁺CCR9⁺ cells in the tumor microenvironment of mice treated with a combination of anti-PD-L1 and E. coli^{pApyr} was dependent on the egress of T cells from the GALT, mice were treated with FTY720 the day before initiating the anti-PD-L1 treatment to block the egress of T cells from the GALT and then scored CD8⁺CCR9⁺ cells in the tumor microenvironment at the end of the experiment.

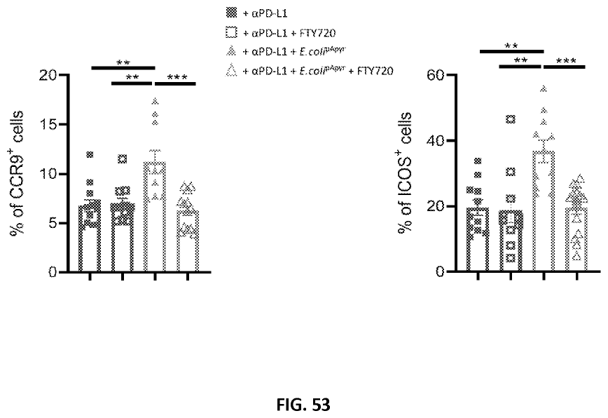

CCR9 expression was analyzed by flow cytometry on electronically gated CD8⁺ TILs as described above in Example 6. As shown in FIG. 53, statistical analysis of the frequencies of CCR9⁺CD8⁺ TILs in tumors from different animals showed the significant increase of these cells in mice treated with a combination of anti-PD-L1 and E. coli^{pApyr} as compared to the group treated with anti-PD-L1, as expected. However, the significant increase of this cell subset was abolished in TILs isolated from MC38 tumors resected from mice in which FTY720 treatment was added to the combination of anti-PD-L1 and E. coli^{pApyr}. Strikingly, also the increase of ICOS expression in CD8⁺ TILs, which characterizes mice treated with a combination of anti-PD-L1 and E. coli^{pApyr} (FIG. 15) was abolished by FTY720 treatment, indicating that the combination of anti-PD-L1 and E. coli^{pApyr} induced the generation of functionally competent cytotoxic T cells in the GALT.

### Example 27: Bacterial Delivery of Apyrase to the Intestine Results in Enhanced IgA Coating of the Ileal Microbiota

Microbiota derived ATP was shown to limit T cell-dependent IgA responses in the Peyer's patches of the small intestine via the ATP-gated ionotropic receptor P2X7, which inhibits T follicular helper (Tfh) cells function and thereby expansion of IgA-secreting plasma cells (Proietti M, Cornacchione V, Rezzonico Jost T, Romagnani A, Faliti C E, Perruzza L, Rigoni R, Radaelli E, Caprioli F, Preziuso S, Brannetti B, Thelen M, McCoy K D, Slack E, Traggiai E, Grassi F. 2014. ATP-gated ionotropic P2X7 receptor controls follicular T helper cell numbers in Peyer's patches to promote host-microbiota mutualism. Immunity 41, 789). In view thereof, it was investigated whether IgA coating of the ileal microbiota was enhanced by administration of E. coli^{pApyr} in tumor bearing mice treated with anti-PD-L1.

The small intestine content was collected, and bacteria isolated by centrifugation and washed to eliminate unbound IgA. Bacterial pellets were resuspended in PBS 5% goat serum, incubated 15 min on ice, centrifuged and resuspended in PBS 1% BSA for staining with APC conjugated rabbit anti-mouse IgA antibodies (Cat. #: SAB1186; Brookwood Biomedical, Birmingham, AL, USA). After 30 min incubation, bacteria were washed twice and analyzed in flow cytometry. Forward and side scatter parameters were used in logarithmic mode. SYTO BC was added to identify bacteria-sized particles containing nucleic acids.

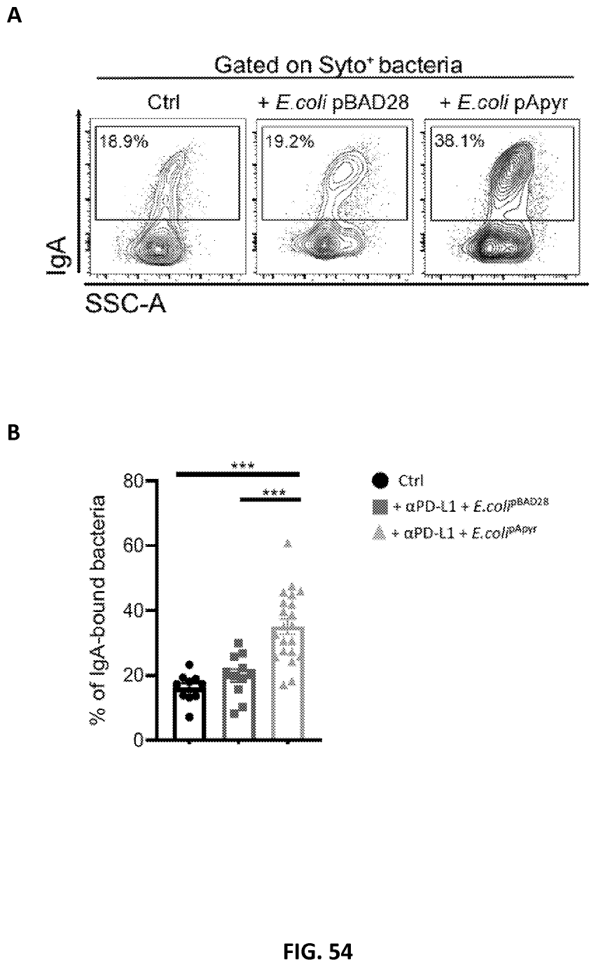

As shown in FIG. 54, flow cytometry and the statistical analysis of the data revealed the significant increase of IgA coated bacteria in the ileum of mice bearing MC38 tumors and treated with a combination of anti-PD-L1 and E. coli^{pApyr}, but not E. coli^{pBAD28}, indicating that administration of apyrase expressing bacteria enhanced the production of secretory IgA recognizing the ileal microbiota.

Without being bound to any theory, the present inventor assumes-based on the finding that administration of apyrase expressing bacteria and anti-PD-L1, but not anti-PD-L1 without apyrase expressing bacteria, enhanced the production of secretory IgA—that apyrase (present in the intestinal lumen) hydrolyzes ATP released by commensal microbiota, which was shown to limit T cell-dependent IgA responses. It is assumed that thereby apyrase can promote the secretory IgA response in the gut of tumor-bearing mice and exert its beneficial effects in combination with the checkpoint inhibitor.

### Example 28: E. coli^{pApyr} Mediated Increase of Ki-67⁺ Cells Among CD8⁺ T Cells in Peyer's Patches of the Ileum Depends on Secretory IgA

The analysis of cell proliferation in the Peyer's patches (PPs) from mice bearing MC38 tumors and treated with anti-PD-L1 showed that E. coli^{pApyr} administration enhanced CD8⁺ cells expansion (FIG. 35). To address whether secretory IgA were important in this phenomenon, electronically gated TCRβ⁺CD8⁺ cells were analyzed by flow cytometry for Ki-67 expression in Peyer's patches from wild-type and IgA^{-/-} MC38 tumor bearing mice treated with anti-PD-L1 or anti-PD-L1 and E. coli^{pApyr}.

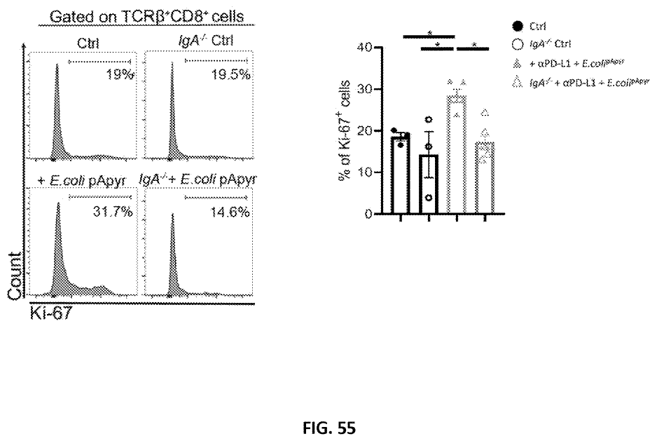

Results are shown in FIG. 55. Strikingly, the increase of Ki-67⁺ cells among electronically gated CD8⁺ T cells isolated from PPs harvested from wild-type mice treated with a combination of anti-PD-L1 and E. coli^{pApyr} was lost in IgA^{-/-} mice. As shown in FIG. 55, statistical analysis of the frequencies of Ki-67⁺CD8⁺ T cells in PPs from different animals showed the significant increase of these cells in wild-type but not IgA^{-/-} mice treated with a combination of anti-PD-L1 and E. coli^{pApyr} as compared to the counterparts treated with stand-alone anti-PD-L1.

### Example 29: E. Coli^{pApyr} Mediated Increase of T-Bet⁺ Cells Among CD8⁺ T Cells in Peyer's Patches of the Ileum Depends on Secretory IgA

The generation and function of effector CD8⁺ T cells relies on the T-box transcription factor T-bet (Tbx21) (Sullivan, B. M., Juedes, A., Szabo, S. J., von Herrath, M., and Glimcher, L. H. 2003. Antigen-driven effector CD8 T cell function regulated by T-bet. Proceedings of the National Academy of Sciences 100, 15818). An effective anti-tumor response during checkpoint blockade treatment depends on T-bet induction that is required for IFN-g production and TILs cytotoxicity (Berrien-Elliott, M. M., Yuan, J., Swier, L. E., Jackson, S. R., Chen, C. L., Donlin, M. J., and Teague, R. M. 2015. Checkpoint Blockade Immunotherapy Relies on T-bet but Not Eomes to Induce Effector Function in Tumor-Infiltrating CD8⁺ T Cells. Cancer Immunology Research 3, 116). As shown in FIG. 36 (Example 18), T-bet⁺ cells among CD8⁺ T cells in the PPs from mice bearing MC38 tumors and treated with anti-PD-L1 were increased by E. coli^{pApyr} administration. To address whether secretory IgA were important in this phenomenon, electronically gated TCRβ⁺CD8⁺ cells were analyzed by flow cytometry for T-bet expression in Peyer's patches from wild-type and IgA^{-/-} MC38 tumor bearing mice treated with anti-PD-L1 or anti-PD-L1 and E. coli^{pApyr}.

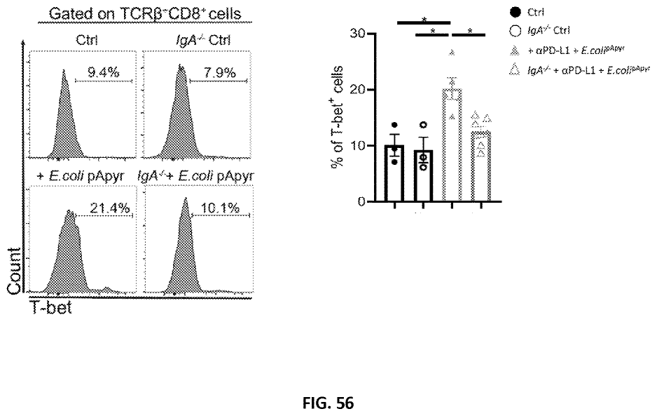

Results are shown in FIG. 56. Strikingly, the increase of T-bet⁺ cells among electronically gated CD8⁺ T cells isolated from PPs harvested from wild-type mice treated with a combination of anti-PD-L1 and E. coli^{pApyr} was lost in IgA^{-/-} mice. As shown in FIG. 56, statistical analysis of the frequencies of T-bet⁺CD8⁺ T cells in PPs from different animals showed the significant increase of these cells in wild-type but not IgA^{-/-} mice treated with a combination of anti-PD-L1 and E. coli^{pApyr} as compared to the counterparts treated with stand-alone anti-PD-L1.

### Example 30: The Improvement of Anti-PD-L1 Treatment by E. coli^{pApyr} in Mice Bearing MC38 Colon Adenocarcinoma Depends on IgA

Secretory IgAs play a crucial role in regulating the composition and function of the commensal microbiota, which in turn condition the intestinal immune system (Weis A. M. and Round J. L. 2021. Microbiota-antibody interactions that regulate gut homeostasis. Cell Host Microbe. 29, 334). To address whether enhanced secretory IgA production was important in promoting the control of tumor growth observed by administration of E. coli^{pApyr} to mice bearing MC38 tumors and treated with anti-PD-L1, IgA deficient mice were used.

To this end, bacteria expressing apyrase (obtained as described in Example 1) were administered in combination with anti-PD-L1 immune checkpoint inhibitor to wild-type and IgA^{-/-} C57BL/6 mice engrafted subcutaneously with colon adenocarcinoma MC38.

The experiments were performed essentially as described in Example 3. Briefly, colon adenocarcinoma MC38 cells were cultured in RPMI-1640 supplemented with 10% heat inactivate fetal bovine serum, 100 U/mL penicillin/streptomycin and 100 U/mL kanamycin. Cells were maintained in 5% CO₂ at 37° C. Tumor cells were harvested at exponential growth and subcutaneously engrafted in 8 week old wild-type or IgA^{-/-} C57Bl/6 mice at 1×10⁶ cells/100 ml (day 0).

Similarly as in Example 3, mice were orally gavaged with E. coli expressing apyrase (E. coli^{pApyr}) in combination with intra-peritoneal administration of anti-PD-L1. Mice were injected intraperitoneally with anti-PD-L1 monoclonal antibody (clone: 10F.9G2; BioXCell) (100 μg/100 μl) at day 8, 11, 14, 17. E. coli^{pApyr} (1×10¹⁰ CFU) was administered daily by orogastric gavage from day 5 to termination of the experiment. Tumor growth was scored with a caliper by measuring the greatest tumor diameter and its perpendicular to determine an average and then the area was calculated as: (average/2)²□.

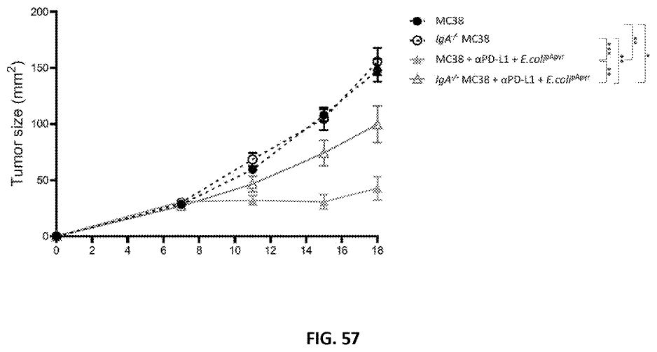

Results are shown in FIG. 57. Lack of IgA resulted in the abrogation of the enhancement of tumor growth control provided in wild-type mice by administration of E. coli^{pApyr} in combination with anti-PD-L1. IgA^{-/-} mice treated with anti-PD-L1 and E. coli^{pApyr} showed an analogous reduction of tumor size compared to untreated mice as observed in mice treated with stand-alone anti-PD-L1, suggesting the therapeutic effect of anti-PD-L1 was not significantly compromised by the absence of IgA. Therefore, the enhancement of secretory IgA production induced by administration of E. coli^{pApyr} is important for enhancing the therapeutic effect of anti-PD-L1 antibody.

### Example 31: The Increase of CCR9⁺ and ICOS⁺ Cells Among CD8⁺ TILs by Administration of E. coli^{pApyr} in Combination with Anti-PD-L1 in Mice Bearing MC38 Colon Adenocarcinoma Depends on IgA

To address whether the increase of CD8⁺CCR9⁺ cells in the tumor microenvironment of mice treated with a combination of anti-PD-L1 and E. coli^{pApyr} was dependent on enhanced secretory IgA production, CCR9⁺ cells were scored among TCRβ⁺CD8⁺ TILs in wild-type and IgA^{-/-} MC38 tumor bearing mice treated with anti-PD-L1 or anti-PD-L1 and E. coli^{pApyr}.

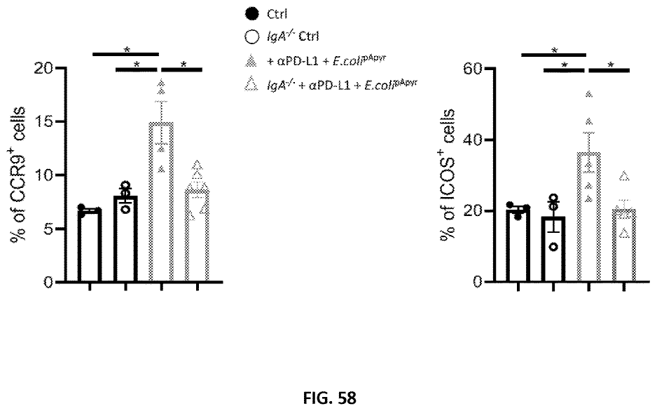

CCR9 expression was analyzed by flow cytometry on electronically gated CD8⁺ TILs as described above in Example 6. As shown in FIG. 58, statistical analysis of the frequencies of CCR9⁺CD8⁺ TILs in tumors from different animals showed the significant increase of these cells in wild-type mice treated with a combination of anti-PD-L1 and E. coli^{pApyr} as compared to the group treated with anti-PD-L1, as expected. However, the significant increase of this cell subset was absent in TILs isolated from IgA mice. Strikingly, also the increase of ICOS expression in CD8⁺ TILs, which characterizes wild-type mice treated with a combination of anti-PD-L1 and E. coli^{pApyr} (FIG. 15) was absent in mice lacking IgA, indicating that the enhanced production of secretory IgA induced by the combination of anti-PD-L1 and E. coli^{pApyr} was important for inducing the generation of functionally competent cytotoxic T cells that infiltrated the tumor microenvironment.

### Example 32: The Frequency of IgA Coated Bacteria in the Ileum Correlates with the Tumor Size in Mice Bearing MC38 Colon Adenocarcinoma and Treated with Anti-PD-L1

To investigate whether IgA coating of commensal microbiota was important in promoting enhanced tumoricidal function of T cells in mice treated with the combination of anti-PD-L1 and E. coli^{pApyr}, the percentage of IgA coated bacteria in the ileum of MC38 tumor bearing mice was correlated with the tumor size at the experimental endpoint.

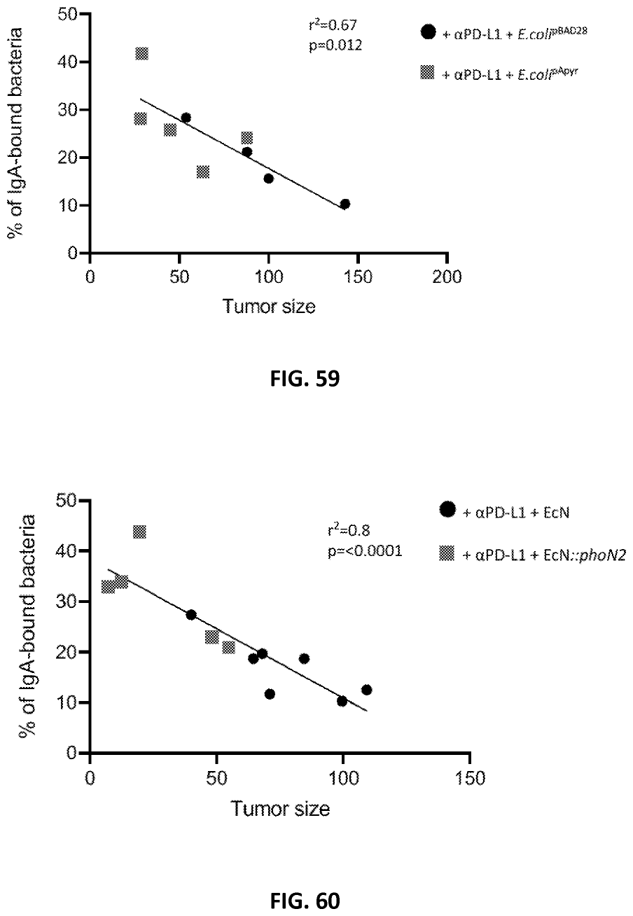

FIG. 59 shows the results of this analysis in MC38 tumor bearing mice treated with anti-PD-L1 and E. coli^{pBAD28} or E. coli^{pApyr}, as described in Example 3, whereas FIG. 60 shows the same analysis in MC38 tumor bearing mice treated with anti-PD-L1 and EcN or Ecn: phoN2, as described in Example 21. In both experimental settings, a negative correlation between the frequency of IgA coated bacteria in the ileum and the tumor size was found, indicating that IgA coating of the ileal microbiota beneficially influenced the competence of the animal to control tumor growth.

### Example 33: The Improvement of Treatment Outcome by E. coli^{pApyr} in Mice Bearing MC38 Colon Adenocarcinoma Depends on Commensal Bacteria Sensitive to Vancomycin

Microbiota composition plays a crucial role in conditioning the responsiveness of cancer patients to immune checkpoint inhibitors. The transplant of fecal microbiota of patients responding to the therapy could convert non-responder patients to responsiveness by inducing favorable changes in immune cell infiltrates and gene expression profiles in both the gut lamina propria and the tumor microenvironment, thereby suggesting that the intestinal microbiota play an important role in regulating the immune response against cancer cells triggered by these biologics (Davar D, Dzutsev A K, McCulloch J A, Rodrigues R R, Chauvin J M, Morrison R M, Deblasio R N, Menna C, Ding Q, Pagliano O, Zidi B, Zhang S, Badger J H, Vetizou M, Cole A M, Fernandes M R, Prescott S, Costa R G F, Balaji A K, Morgun A, Vujkovic-Cvijin I, Wang H, Borhani A A, Schwartz M B, Dubner H M, Ernst S J, Rose A, Najjar Y G, Belkaid Y, Kirkwood J M, Trinchieri G, Zarour H M. 2021. Fecal microbiota transplant overcomes resistance to anti-PD-1 therapy in melanoma patients. Science 371, 595. Baruch E N, Youngster I, Ben-Betzalel G, Ortenberg R, Lahat A, Katz L, Adler K, Dick-Necula D, Raskin S, Bloch N, Rotin D, Anafi L, Avivi C, Melnichenko J, Steinberg-Silman Y, Mamtani R, Harati H, Asher N, Shapira-Frommer R, Brosh-Nissimov T, Eshet Y, Ben-Simon S, Ziv O, Khan M A W, Amit M, Ajami N J, Barshack I, Schachter J, Wargo J A, Koren O, Markel G, Boursi B. 2021. Fecal microbiota transplant promotes response in immunotherapy-refractory melanoma patients. Science 371, 602).

To address if the therapeutic improvement induced by combining E. coli^{pApyr} to anti-PD-L1 was dependent on the intestinal microbiota, Vancomycin was administered to deplete the intestinal microbiota of MC38 tumor bearing mice treated with anti-PD-L1 or anti-PD-L1 and E. coli^{pBAD28} or anti-PD-L1 and E. coli^{pApyr}.

Bacteria expressing apyrase (obtained as described in Example 1) were administered in combination with anti-PD-L1 immune checkpoint inhibitor to mice engrafted subcutaneously with colon adenocarcinoma MC38. The experiments were performed essentially as described in Example 3 with the difference that mice were pre-treated for 15 d before tumor engraftment with Vancomycin (200 mg/L) in drinking water; since E. coli is resistant to vancomycin, the antibiotic was maintained in the drinking water until the end of the experiment. Briefly, colon adenocarcinoma MC38 cells were cultured in RPMI-1640 supplemented with 10% heat inactivate fetal bovine serum, 100 U/mL penicillin/streptomycin and 100 U/mL kanamycin. Cells were maintained in 5% CO₂ at 37° C. Tumor cells were harvested at exponential growth and subcutaneously engrafted in 8 week old C57Bl/6 mice at 1×10⁶ cells/100 ml (day 0).

Similarly as in Example 3, mice were orally gavaged with E. coli expressing apyrase (E. coli^{pApyr}) or E. coli transformants bearing the empty plasmid (E. coli_{pBAD28}) in combination with intra-peritoneal administration of anti-PD-L1. Mice were injected intraperitoneally with anti-PD-L1 monoclonal antibody (clone: 10F.9G2; BioXCell) (100 μg/100 μl) at day 8, 11, 14, 17. E. coli^{pApyr} or E. coli^{pBAD28} (1×10¹⁰ CFU) was administered daily by orogastric gavage from day 5 to termination of the experiment. Tumor growth was scored with a caliper by measuring the greatest tumor diameter and its perpendicular to determine an average and then the area was calculated as: (average/2)²□.

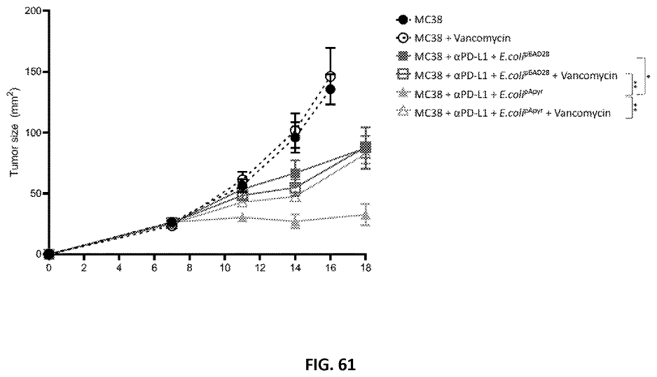

Results are shown in FIG. 61. Whereas administration of Vancomycin did not affect the response to anti-PD-L1 in mice gavaged either with PBS or E. coli^{pBAD28}, it completely abolished the enhancement of tumor growth control provided by administration of E. coli^{pApyr} in combination with anti-PD-L1. These results indicate that Vancomycin sensitive bacteria did not affect the response to anti-PD-L1 but were required for implementing the beneficial effect of E. coli^{pApyr} on the control of tumor growth.

### Example 34: IgA Coated Bacteria in the Ileum of Mice Treated with E. coli^{pApyr} are Sensitive to Vancomycin

In view of the relevance of IgA for the therapeutic effect of E. coli^{pApyr} in combination with anti-PD-L1, it was addressed next, whether administration of Vancomycin affected the abundance of IgA coated bacteria in the ileum.

The small intestine content was collected and bacteria isolated by centrifugation and washed to eliminate unbound IgA. Bacterial pellets were resuspended in PBS 5% goat serum, incubated 15 min on ice, centrifuged and resuspended in PBS 1% BSA for staining with APC conjugated rabbit anti-mouse IgA antibodies (Cat. #: SAB1186; Brookwood Biomedical, Birmingham, AL, USA). After 30 min incubation, bacteria were washed twice and analysed in flow cytometry. Forward and side scatter parameters were used in logarithmic mode. SYTO BC was added to identify bacteria-sized particles containing nucleic acids.

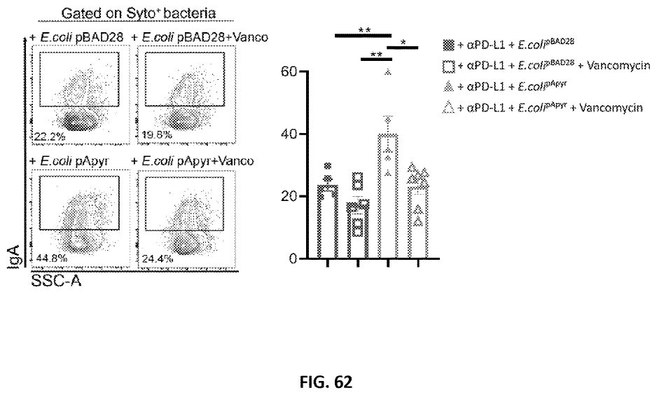

As shown in FIG. 62, flow cytometry and the statistical analysis of the data revealed that Vancomycin induced the significant depletion of IgA coated bacteria in the ileum of mice bearing MC38 tumors and treated with a combination of anti-PD-L1 and E. coli^{pApyr}, whereas IgA coating of bacteria in mice treated with E. coli^{pBAD28} was not significantly affected by Vancomycin. Since IgA are required for the improved control of tumor growth induced by E. coli^{pApyr} in combination with anti-PD-L1, these results indicate that E. coli^{pApyr} enhances the production of secretory IgA targeting Vancomycin-sensitive commensal bacteria that mediate the therapeutic effect of apyrase.

TABLE OF SEQUENCES AND SEQ ID NUMBERS (SEQUENCE LISTING):

| SEQ ID NO | Sequence | Remarks |
|---|---|---|
| SEQ ID NO: 1 | MKTKNFLLFCIATNMIFIPSANALKAEGFLTQQ | Apyrase |
| | TSPDSLSILPPPPAEDSVVFLADKAHYEFGRS | |
| | LRDANRVRLASEDAYYENFGLAFSDAYGMDI | |
| | SRENTPILYQLLTQVLQDSHDYAVRNAKEYYK | |
| | RVRPFVIYKDATCTPDKDEKMAITGSYPSGHA | |
| | SFGWAVALILAEINPQRKAEILRRGYEFGESR | |
| | VICGAHWQSDVEAGRLMGASVVAVLHNTPE | |
| | FTKSLSEAKKEFEELNTPTNELTP | |
| SEQ ID NO: 2 | MKTKNFLLFCIATNMIFIPSANALKAEGFLTQQ | Loss-of-function |
| | TSPDSLSILPPPPAEDSVVFLADKAHYEFGRS | isoform of apyrase |
| | LRDANRVRLASEDAYYENFGLAFSDAYGMDI | |
| | SRENTPILYQLLTQVLQDSHDYAVRNAKEYYK | |
| | RVRPFVIYKDATCTPDKDEKMAITGSYPSGHA | |
| | SFGWAVALILAEINPQRKAEILRRGYEFGESP | |
| | VICGAHWQSDVEAGRLMGASVVAVLHNTPE | |
| | FTKSLSEAKKEFEELNTPTNELTP | |
| SEQ ID NO: 3 | ATGAAAACCAAAAACTTTCTTCTTTTTTGTA | phoN2 gene |
| | TTGCTACAAATATGATTTTTATCCCCTCAGC | encoding apyrase |
| | AAATGCTCTGAAGGCAGAAGGTTTTCTCAC | |
| | TCAACAAACTTCACCAGACAGTTTGTCAATA | |
| | CTTCCGCCGCCTCCGGCAGAGGATTCAGT | |
| | AGTATTTCTGGCTGACAAAGCTCATTATGA | |
| | ATTCGGCCGCTCGCTCCGGGATGCTAATC | |
| | GTGTACGTCTCGCTAGCGAAGATGCATACT | |
| | ACGAGAATTTTGGTCTTGCATTTTCAGATG | |
| | CTTATGGCATGGATATTTCAAGGGAAAATA | |
| | CCCCAATCTTATATCAGTTGTTAACACAAGT | |
| | ACTACAGGATAGCCATGATTACGCCGTGCG | |
| | TAACGCCAAAGAATATTATAAAAGAGTTCGT | |
| | CCATTCGTTATTTATAAAGACGCAACCTGTA | |
| | CACCTGATAAAGATGAGAAAATGGCTATCA | |
| | CTGGCTCTTATCCCTCTGGTCATGCATCCT | |
| | TTGGTTGGGCAGTAGCACTGATACTTGCGG | |
| | AGATTAATCCTCAACGTAAAGCGGAAATAC | |
| | TTCGACGTGGATATGAGTTTGGAGAAAGTC | |
| | GGGTCATCTGCGGTGCGCATTGGCAAAGC | |
| | GATGTAGAGGCTGGGCGTTTAATGGGAGC | |
| | ATCGGTTGTTGCAGTACTTCATAATACACCT | |
| | GAATTTACCAAAAGCCTTAGCGAAGCCAAA | |
| | AAAGAGTTTGAAGAATTAAATACTCCTACCA | |
| | ATGAACTGACCCCATAA | |
| SEQ ID NO: 4 | CGAGCAGGCACACTGGAAGTATTGCTGCAT | EcN malP gene |
| | CAGGCGCAGCTTTTTACCGGCAGTATGGTT | portion |
| | GTCGTTTGGATAGAGAACTTTGGTCAGTTTT | |
| | TCCGCGTTGATGCCCTGCTGTTCGGCACG | |
| | CAGGAAATCACCGTCGTTAAATTTAGTCAG | |
| | ATCAAACGGATGCGCATGCGTCGCCTGCC | |
| | ACAGACGCAGTGGCTGCGCCACGCCATTA | |
| | CGATAGCCGACAACGGGGAGATCCCACGC | |
| | TTGACCGGTAATGGTAAACTCCGGCTCCCA | |
| | GCGTCCATCTTTCGTCACTTTACCGCCAAT | |
| | CCCTACCTGCACATCCAGTGCTTCGTTGTG | |
| | GCGGAACCACGGGTAGTTACCGCGATGCC | |
| | AGTCATCCGGCGCTTCAACCTGTTTGCCAT | |
| | CGACAAATGACTGGCGGAACAAGCCATATT | |
| | GATAATTAAGGCCGTAGCCAGTAGCTGACT | |
| | GCCCGACAGTTGCCATTGAGTCGAGGAAG | |
| | CACGCCGCCAGACGTCCCAGACCACCGTT | |
| | CCCCAGCGCCGGGTCGATCTCTTCTTCCAA | |
| | CAGGTCAGTCAGGTTGATGTCATAAGCCTT | |
| | CAACGAATCCTGTACATCCTGATACCAGCC | |
| | GAGATTCAACAGGTTGTTGCCCGTCAGGCG | |
| | ACCAATCAAAAACTCCATTGAGATGTAGTTA | |
| | ACATGTCGCTGATTCGCCACTGGCTTGGCG | |
| | AATGGCTGAGCACGCAGCATTTCGGCCAGT | |
| | GCTTCGCTCACTGCCAGCCACCACTGGCG | |
| | AGGAGTCATTTCAGCCGCAGAATTTAAGCC | |
| | ATAACGCTGCCACTGACGTGAAAGCGCTTC | |
| | CTGAAATTGCTTATCGTTAAAAATAGGTTGT | |
| SEQ ID NO: 5 | ATGCTGATTCCGTCAAAATTAAGTCGTCCG | EcN malT gene |
| | GTTCGACTCGACCATACCGTGGTTCGTGAG | portion |
| | CGCCTGCTGGCTAAACTTTCCGGCGCGAA | |
| | CAACTTCCGGCTGGCGCTGATCACAAGTCC | |
| | TGCGGGCTACGGAAAGACCACGCTCATTTC | |
| | CCAGTGGGCGGCAGGCAAAAACGATATCG | |
| | GCTGGTACTCGCTGGATGAAGGTGATAACC | |
| | AGCAAGAGCGTTTCGCCAGCTATCTCATTG | |
| | CCGCCGTGCAACAGGCAACCAACGGTCAC | |
| | TGCGCGATATGTGAGACGATGGCGCAAAAA | |
| | CGGCAATATGCCAGCCTGACGTCACTCTTC | |
| | GCCCAGCTTTTCATTGAGCTGGCGGAATGG | |
| | CATAGCCCACTTTATCTGGTCATCGATGAC | |
| | TATCATCTGATCACTAATCCTGTGATCCACG | |
| | AGTCAATGCGCTTCTTTATTCGCCATCAACC | |
| | AGAAAATCTCACCCTTGTGGTGTTGTCACG | |
| | CAACCTTCCGCAACTGGGCATTGCCAATCT | |
| | GCGTGTTCGTCCAGCTAGCGAATTCGCTGG | |
| | AAATTGGCAGTCAGCAACTGGCATTTACCC | |
| | ATCAGGAAGCGAAGCAGTTTTTTGATTGCC | |
| | GTCTGTCATCGCCGATTGAAGCTGCAGAAA | |
| | GCAGTCGGATTTGTGATGATGTTTCCGGTT | |
| | GGGCGACGGCACTGCAGCTAATCGCCCTC | |
| | TCCGCCCGGCAGAATACTCACTCAGCCCAT | |
| | AAGTCGGCACGCCGCCTGGCGGGAATCAA | |
| | TGCCAGCCATCTTTCGGATTATCTGGTCGA | |
| | TGAGGTTTTGGATAACGTCGATCTCGCAAC | |
| | GCGCCA | |
| SEQ ID NO: 6 | CAGCTAACACCACGTCGTCCCTATCTGCTG | DNA fragment |
| | CCCTAGGTCTATGAGTGGTTGCTGGATAAC | including the P_{proD} |
| | TTTACGGGCATGCATAAGGCTCGTATAATA | promoter, the |
| | TATTCAGGGAGACCACAACGGTTTCCCTCT | BBa_BB0032 RBS, |
| | ACAAATAATTTTGTTTAACTTTTACTAGAGT | the S. flexneri phoN2 |
| | CACACAGGAAAGTACTAGATGAAAACCAAA | gene and the phoN2 |
| | AACTTTCTTCTTTTTTGTATTGCTACAAATAT | transcriptional |
| | GATTTTTATCCCCTCAGCAAATGCTCTGAA | terminator |
| | GGCAGAAGGTTTTCTCACTCAACAAACTTC | |
| | ACCAGACAGTTTGTCAATACTTCCGCCGCC | |
| | TCCGGCAGAGGATTCAGTAGTATTTCTGGC | |
| | TGACAAAGCTCATTATGAATTCGGCCGCTC | |
| | GCTCCGGGATGCTAATCGTGTACGTCTCG | |
| | CTAGCGAAGATGCATACTACGAGAATTTTG | |
| | GTCTTGCATTTTCAGATGCTTATGGCATGG | |
| | ATATTTCAAGGGAAAATACCCCAATCTTATA | |
| | TCAGTTGTTAACACAAGTACTACAGGATAG | |
| | CCATGATTACGCCGTGCGTAACGCCAAAGA | |
| | ATATTATAAAAGAGTTCGTCCATTCGTTATT | |
| | TATAAAGACGCAACCTGTACACCTGATAAA | |
| | GATGAGAAAATGGCTATCACTGGCTCTTAT | |
| | CCCTCTGGTCATGCATCCTTTGGTTGGGCA | |
| | GTAGCACTGATACTTGCGGAGATTAATCCT | |
| | CAACGTAAAGCGGAAATACTTCGACGTGGA | |
| | TATGAGTTTGGAGAAAGTCGGGTCATCTGC | |
| | GGTGCGCATTGGCAAAGCGATGTAGAGGC | |
| | TGGGCGTTTAATGGGAGCATCGGTTGTTGC | |
| | AGTACTTCATAATACACCTGAATTTACCAAA | |
| | AGCCTTAGCGAAGCCAAAAAAGAGTTTGAA | |
| | GAATTAAATACTCCTACCAATGAACTGACC | |
| | CCATAAAGCTGGACAGCCTGTATCAGGCT | |
| | ATGGAGGGCCCATAGACAAATCTACCCTA | |
| | TATGAGCATAGGAGGAGTCTATGGGCACA | |
| | CCACGTTTTACCCCTGAATTTAAGGGATTA | |
| | CTGGAAAGGCTGGGACATATCCCTCCGGC | |
| | AGAAGCAGAAAAAGCTTATTATGCTGCCA | |
| | TCGGAAACGATGATCTGGCAACCTGAGTT | |
| | CACAGATAAAACATTCTCTAGGAAACTCG | |
| | GGGCGGTTCCGTTCACCACATGCAATGTG | |
| | GTGTTGCAGGGGAACGGTCTGCCCATCCC | |
| | CTATGTCGATCAATATAACAGAAATGACA | |
| | ACTTCAGATTCAGGGCACAACCTAAATAT | |
| | ATTTTAGGTCACCTCTCAAATCGTTTGCCT | |
| | GA | |
| SEQ ID NO: 7 | GAAGTTCCTATTCTCTAGAAAGTATAGGAAC | DNA fragment |
| | TTCGGCGCGCCTACCTGTGACGGAAGATC | including the E. coli |
| | ACTTCGCAGAATAAATAAATCCTGGTGTCC | cat gene flanked by |
| | CTGTTGATACCGGGAAGCCCTGGGCCAAC | the FRT sequences |
| | TTTTGGCGAAAATGAGACGTTGATCGGCAC | |
| | GTAAGAGGTTCCAACTTTCACCATAATGAAA | |
| | TAAGATCACTACCGGGCGTATTTTTTGAGTT | |
| | GTCGAGATTTTCAGGAGCTAAGGAAGCTAA | |
| | AATGGAGAAAAAAATCACTGGATATACCAC | |
| | CGTTGATATATCCCAATGGCATCGTAAAGA | |
| | ACATTTTGAGGCATTTCAGTCAGTTGCTCAA | |
| | TGTACCTATAACCAGACCGTTCAGCTGGAT | |
| | ATTACGGCCTTTTTAAAGACCGTAAAGAAAA | |
| | ATAAGCACAAGTTTTATCCGGCCTTTATTCA | |
| | CATTCTTGCCCGCCTGATGAATGCTCATCC | |
| | GGAATTACGTATGGCAATGAAAGACGGTGA | |
| | GCTGGTGATATGGGATAGTGTTCACCCTTG | |
| | TTACACCGTTTTCCATGAGCAAACTGAAAC | |
| | GTTTTCATCGCTCTGGAGTGAATACCACGA | |
| | CGATTTCCGGCAGTTTCTACACATATATTCG | |
| | CAAGATGTGGCGTGTTACGGTGAAAACCTG | |
| | GCCTATTTCCCTAAAGGGTTTATTGAGAATA | |
| | TGTTTTTCGTCTCAGCCAATCCCTGGGTGA | |
| | GTTTCACCAGTTTTGATTTAAACGTGGCCAA | |
| | TATGGACAACTTCTTCGCCCCCGTTTTCAC | |
| | CATGGGCAAATATTATACGCAAGGCGACAA | |
| | GGTGCTGATGCCGCTGGCGATTCAGGTTC | |
| | ATCATGCCGTTTGTGATGGCTTCCATGTCG | |
| | GCAGAATGCTTAATGAATTACAACAGTACT | |
| | GCGATGAGTGGCAGGGCGGGGCGTAAGG | |
| | CGCGCCATTTAAATGAAGTTCCTATTCCGA | |
| | AGTTCCTATTCTCTAGAAAGTATAGGAACTT | |
| | CGAAGCAGCTCCAGCCTACACAATGAATTC | |
| SEQ ID NO: 8 | ATGGGCTCCATCGGTGCAGCAAGCATGGA | cDNA encoding |
| | ATTTTGTTTTGATGTATTCAAGGAGCTCAAA | chicken ovalbumin |
| | GTCCACCATGCCAATGAGAACATCTTCTAC | |
| | TGCCCCATTGCCATCATGTCAGCTCTAGCC | |
| | ATGGTATACCTGGGTGCAAAAGACAGCACC | |
| | AGGACACAAATAAATAAGGTTGTTCGCTTT | |
| | GATAAACTTCCAGGATTCGGAGACAGTATT | |
| | GAAGCTCAGTGTGGCACATCTGTAAACGTT | |
| | CACTCTTCACTTAGAGACATCCTCAACCAAA | |
| | TCACCAAACCAAATGATGTTTATTCGTTCAG | |
| | CCTTGCCAGTAGACTTTATGCTGAAGAGAG | |
| | ATACCCAATCCTGCCAGAATACTTGCAGTG | |
| | TGTGAAGGAACTGTATAGAGGAGGCTTGGA | |
| | ACCTATCAACTTTCAAACAGCTGCAGATCAA | |
| | GCCAGAGAGCTCATCAATTCCTGGGTAGAA | |
| | AGTCAGACAAATGGAATTATCAGAAATGTC | |
| | CTTCAGCCAAGCTCCGTGGATTCTCAAACT | |
| | GCAATGGTTCTGGTTAATGCCATTGTCTTCA | |
| | AAGGACTGTGGGAGAAAGCATTTAAGGATG | |
| | AAGACACACAAGCAATGCCTTTCAGAGTGA | |
| | CTGAGCAAGAAAGCAAACCTGTGCAGATGA | |
| | TGTACCAGATTGGTTTATTTAGAGTGGCATC | |
| | AATGGCTTCTGAGAAAATGAAGATCCTGGA | |
| | GCTTCCATTTGCCAGTGGGACAATGAGCAT | |
| | GTTGGTGCTGTTGCCTGATGAAGTCTCAGG | |
| | CCTTGAGCAGCTTGAGAGTATAATCAACTTT | |
| | GAAAAACTGACTGAATGGACCAGTTCTAAT | |
| | GTTATGGAAGAGAGGAAGATCAAAGTGTAC | |
| | TTACCTCGCATGAAGATGGAGGAAAAATAC | |
| | AACCTCACATCTGTCTTAATGGCTATGGGC | |
| | ATTACTGACGTGTTTAGCTCTTCAGCCAATC | |
| | TGTCTGGCATCTCCTCAGCAGAGAGCCTGA | |
| | AGATATCTCAAGCTGTCCATGCAGCACATG | |
| | CAGAAATCAATGAAGCAGGCAGAGAGGTG | |
| | GTAGGGTCAGCAGAGGCTGGAGTGGATGC | |
| | TGCAAGCGTCTCTGAAGAATTTAGGGCTGA | |
| | CCATCCATTCCTCTTCTGTATCAAGCACATC | |
| | GCAACCAACGCCGTTCTCTTCTTTGGCAGA | |
| | TGTGTTTCCCCT | |
| SEQ ID NO: 9 | MGSIGAASMEFCFDVFKELKVHHANENIFYC | chicken ovalbumin |
| | PIAIMSALAMVYLGAKDSTRTQINKVVRFDKL | |
| | PGFGDSIEAQCGTSVNVHSSLRDILNQITKPN | |
| | DVYSFSLASRLYAEERYPILPEYLQCVKELYR | |
| | GGLEPINFQTAADQARELINSWVESQTNGIIR | |
| | NVLQPSSVDSQTAMVLVNAIVFKGLWEKAFK | |
| | DEDTQAMPFRVTEQESKPVQMMYQIGLFRV | |
| | ASMASEKMKILELPFASGTMSMLVLLPDEVS | |
| | GLEQLESIINFEKLTEWTSSNVMEERKIKVYLP | |
| | RMKMEEKYNLTSVLMAMGITDVFSSSANLSG | |
| | ISSAESLKISQAVHAAHAEINEAGREVVGSAE | |
| | AGVDAASVSEEFRADHPFLFCIKHIATNAVLF | |
| | FGRCVSP | |

1. A method for reducing the risk of occurrence, treating, ameliorating, or reducing cancer or initiating, enhancing or prolonging an anti-tumor-response in a subject in need thereof, comprising administering to the subject a combination of
(i) an immune checkpoint modulator; and
(ii) (a) an ATP hydrolyzing enzyme,
(b) a nucleic acid comprising a polynucleotide encoding the ATP hydrolyzing enzyme,
(c) a host cell comprising the nucleic acid,
(d) a microorganism comprising the nucleic acid, or
(e) a viral particle comprising the nucleic acid
wherein the ATP hydrolyzing enzyme is apyrase.

2. The method of claim 1, wherein the ATP hydrolyzing enzyme is not endogenous CD39.

3. The method of claim 1, wherein the ATP hydrolyzing enzyme is a soluble ATP hydrolyzing enzyme.

4. The method of claim 1, wherein the apyrase is a bacterial apyrase or a plant apyrase.

5. The method of claim 1, wherein the ATP hydrolyzing enzyme comprises an amino acid sequence as set forth in SEQ ID NO: 1 or a sequence variant thereof having at least 70%, 80% or 90% sequence identity.

6. The method of claim 1, wherein the method comprises administering to the subject a combination of
(i) the immune checkpoint modulator; and
(ii) the nucleic acid comprising the polynucleotide encoding the ATP hydrolyzing enzyme.

7. The method of claim 6, wherein the nucleic acid comprising the polynucleotide encoding the ATP hydrolyzing enzyme is a vector.

8. The method of claim 6, wherein the nucleic acid further comprises heterologous elements for (heterologous) expression of the ATP hydrolyzing enzyme.

9. The method of claim 1, wherein the method comprises administering to the subject a combination of
(i) the immune checkpoint modulator; and
(ii) the host cell comprising the nucleic acid.

10. The method of claim 9, wherein the host cell is a prokaryotic or a eukaryotic cell.

11. The method of claim 1, wherein the method comprises administering to the subject a combination of
(i) the immune checkpoint modulator; and
(ii) the microorganism comprising the nucleic acid.

12. The method of claim 11, wherein the microorganism is selected from the group consisting of archaea, bacteria and eukaryotes.

13. The method of claim 11, wherein the microorganism is selected from the group consisting of Escherichia spp., Salmonella spp., Yersinia spp., Vibrio spp., Listeria spp., Lactococcus spp., Shigella spp., Cyanobacteria, and Saccharomyces spp.

14. The method of claim 11, wherein the microorganisms are provided as probiotics.

15. The method of claim 11, wherein the virulence of the microorganism is attenuated.

16. The method of claim 11, wherein the microorganism is a (recombinant) bacterium comprising the nucleic acid comprising the polynucleotide encoding the ATP hydrolyzing enzyme.

17. The method of claim 16, wherein the bacterium heterologously expresses the ATP hydrolyzing enzyme.

18. The method of claim 16, wherein the bacterium is selected from the group consisting of Gram-positive bacteria, Gram-negative bacteria and Cyanobacteria.

19. The method of claim 16, wherein the bacterium is selected from the group consisting of Escherichia coli, Salmonella typhi, Salmonella typhimurium, Yersinia enterocolitica, Vibrio cholerae, Listeria monocytogenes, Lactococcus lactis and Shigella flexneri.

20. The method of claim 16, wherein the bacterium is E. coli of the strain Nissle 1917.

21. The method of claim 1, wherein the method comprises administering to the subject a combination of
(i) the immune checkpoint modulator; and
(ii) the viral particle comprising the nucleic acid.

22. The method of claim 21, wherein the viral particle is a bacteriophage.

23. The method of claim 1, wherein combination is comprised in a composition.

24. The method of claim 23, wherein the composition is a pharmaceutical composition further comprising a pharmaceutically acceptable carrier, diluent and/or excipient.

25. The method of claim 23, wherein the composition comprises a periplasmic extract of a bacterium comprising the nucleic acid comprising the polynucleotide encoding the ATP hydrolyzing enzyme.

26. The method of claim 1, wherein the immune checkpoint modulator is an inhibitor of an inhibitory checkpoint molecule (checkpoint inhibitor).

27. The method of claim 26, wherein the inhibitory checkpoint molecule is selected from the group consisting of A2AR, B7-H3, B7-H4, BTLA, CD40, CTLA-4, IDO, KIR, LAG3, PD-1, PDL-1, PD-L2, TIM-3, VISTA, CEACAM1, GARP, PS, CSF1R, CD94/NKG2A, TDO, TNFR, TIGIT and FasR/DcR3.

28. The method of claim 1, wherein the immune checkpoint modulator is an inhibitor of A2AR, B7-H3, B7-H4, BTLA, CD40, CTLA-4, IDO, KIR, LAG3, PD-1, TIM-3, VISTA, CEACAM1, GARP, PS, CSF1R, CD94/NKG2A, TDO, TNFR, TIGIT or DcR3; or an inhibitor of a ligand thereof.

29. The method of claim 1, wherein the immune checkpoint modulator is an inhibitor of the CTLA-4 pathway or the PD-1 pathway.

30. The method of claim 1, wherein the immune checkpoint modulator is an inhibitor of PD-1, PD-L1, or PD-L2.

31. The method of claim 1, wherein the combination further comprises
(a) an antigen or a fragment thereof comprising at least one antigenic epitope,
(b) a nucleic acid comprising a polynucleotide encoding the antigen or the fragment thereof comprising at least one antigenic epitope,
(c) a host cell comprising the nucleic acid comprising the polynucleotide encoding the antigen or the fragment thereof comprising at least one antigenic epitope,
(d) a microorganism comprising the nucleic acid comprising the polynucleotide encoding the antigen or the fragment thereof comprising at least one antigenic epitope, or
(e) a viral particle comprising the nucleic acid comprising the polynucleotide encoding the antigen or the fragment thereof comprising at least one antigenic epitope.

32. The method of claim 31, wherein the combination comprises the host cell or the microorganism comprising the nucleic acid comprising the polynucleotide encoding the ATP hydrolyzing enzyme and the host cell or the microorganism comprising the nucleic acid comprising the polynucleotide encoding the antigen or the fragment thereof comprising at least one antigenic epitope.

33. The method of claim 31, wherein the combination comprises the host cell or the microorganism (heterologously) expressing the ATP hydrolyzing enzyme and the host cell or the microorganism (heterologously) expressing the antigen or the fragment thereof comprising at least one antigenic epitope.

34. The method of claim 1, wherein the combination is administered via a parenteral route of administration.

35. The method of claim 1, wherein the combination is orally administered.

36. A combination of
(i) an immune checkpoint modulator; and
(ii) an ATP hydrolyzing enzyme, wherein the ATP hydrolyzing enzyme is apyrase,
(iii) a nucleic acid comprising a polynucleotide encoding the ATP hydrolyzing enzyme,
(iv) a host cell comprising the nucleic acid,
(v) a microorganism comprising the nucleic acid, or
(vi) a viral particle comprising the nucleic acid.

37. A kit comprising:
(i) an immune checkpoint modulator; and
(ii) (a) an ATP hydrolyzing enzyme,
(b) a nucleic acid comprising a polynucleotide encoding the ATP hydrolyzing enzyme,
(c) a host cell comprising the nucleic acid,
(d) a microorganism comprising the nucleic acid, or
(e) a viral particle comprising the nucleic acid,
wherein the ATP hydrolyzing enzyme is apyrase.


SEQUENCE LISTING (9 sequences)

SEQ ID NO: 1 - protein, 246 aa, Shigella flexneri
MKTKNFLLFCIATNMIFIPSANALKAEGFLTQQTSPDSLSILPPPPAEDSVVFLADKAHYEFGRSLRDANRVRLASEDAYYENFGLAFSDAYGMDISRENTPILYQLLTQVLQDSHDYAVRNAKEYYKRVRPFVIYKDATCTPDKDEKMAITGSYPSGHASFGWAVALILAEINPQRKAEILRRGYEFGESRVICGAHWQSDVEAGRLMGASVVAVLHNTPEFTKSLSEAKKEFEELNTPTNELTP

SEQ ID NO: 2 - protein, 246 aa, synthetic construct
  REGION 1..246: note = Loss-of-function isoform of apyrase
MKTKNFLLFCIATNMIFIPSANALKAEGFLTQQTSPDSLSILPPPPAEDSVVFLADKAHYEFGRSLRDANRVRLASEDAYYENFGLAFSDAYGMDISRENTPILYQLLTQVLQDSHDYAVRNAKEYYKRVRPFVIYKDATCTPDKDEKMAITGSYPSGHASFGWAVALILAEINPQRKAEILRRGYEFGESPVICGAHWQSDVEAGRLMGASVVAVLHNTPEFTKSLSEAKKEFEELNTPTNELTP

SEQ ID NO: 3 - DNA, 741 bp, Shigella flexneri
  source 1..741: mol_type = other DNA
atgaaaaccaaaaactttcttcttttttgtattgctacaaatatgatttttatcccctcagcaaatgctctgaaggcagaaggttttctcactcaacaaacttcaccagacagtttgtcaatacttccgccgcctccggcagaggattcagtagtatttctggctgacaaagctcattatgaattcggccgctcgctccgggatgctaatcgtgtacgtctcgctagcgaagatgcatactacgagaattttggtcttgcattttcagatgcttatggcatggatatttcaagggaaaataccccaatcttatatcagttgttaacacaagtactacaggatagccatgattacgccgtgcgtaacgccaaagaatattataaaagagttcgtccattcgttatttataaagacgcaacctgtacacctgataaagatgagaaaatggctatcactggctcttatccctctggtcatgcatcctttggttgggcagtagcactgatacttgcggagattaatcctcaacgtaaagcggaaatacttcgacgtggatatgagtttggagaaagtcgggtcatctgcggtgcgcattggcaaagcgatgtagaggctgggcgtttaatgggagcatcggttgttgcagtacttcataatacacctgaatttaccaaaagccttagcgaagccaaaaaagagtttgaagaattaaatactcctaccaatgaactgaccccataa

SEQ ID NO: 4 - DNA, 840 bp, synthetic construct
  misc_feature 1..840: note = EcN malP gene portion
  source 1..840: mol_type = other DNA
cgagcaggcacactggaagtattgctgcatcaggcgcagctttttaccggcagtatggttgtcgtttggatagagaactttggtcagtttttccgcgttgatgccctgctgttcggcacgcaggaaatcaccgtcgttaaatttagtcagatcaaacggatgcgcatgcgtcgcctgccacagacgcagtggctgcgccacgccattacgatagccgacaacggggagatcccacgcttgaccggtaatggtaaactccggctcccagcgtccatctttcgtcactttaccgccaatccctacctgcacatccagtgcttcgttgtggcggaaccacgggtagttaccgcgatgccagtcatccggcgcttcaacctgtttgccatcgacaaatgactggcggaacaagccatattgataattaaggccgtagccagtagctgactgcccgacagttgccattgagtcgaggaagcacgccgccagacgtcccagaccaccgttccccagcgccgggtcgatctcttcttccaacaggtcagtcaggttgatgtcataagccttcaacgaatcctgtacatcctgataccagccgagattcaacaggttgttgcccgtcaggcgaccaatcaaaaactccattgagatgtagttaacatgtcgctgattcgccactggcttggcgaatggctgagcacgcagcatttcggccagtgcttcgctcactgccagccaccactggcgaggagtcatttcagccgcagaatttaagccataacgctgccactgacgtgaaagcgcttcctgaaattgcttatcgttaaaaataggttgtgacat

SEQ ID NO: 5 - DNA, 813 bp, synthetic construct
  misc_feature 1..813: note = EcN malT gene portion
  source 1..813: mol_type = other DNA
atgctgattccgtcaaaattaagtcgtccggttcgactcgaccataccgtggttcgtgagcgcctgctggctaaactttccggcgcgaacaacttccggctggcgctgatcacaagtcctgcgggctacggaaagaccacgctcatttcccagtgggcggcaggcaaaaacgatatcggctggtactcgctggatgaaggtgataaccagcaagagcgtttcgccagctatctcattgccgccgtgcaacaggcaaccaacggtcactgcgcgatatgtgagacgatggcgcaaaaacggcaatatgccagcctgacgtcactcttcgcccagcttttcattgagctggcggaatggcatagcccactttatctggtcatcgatgactatcatctgatcactaatcctgtgatccacgagtcaatgcgcttctttattcgccatcaaccagaaaatctcacccttgtggtgttgtcacgcaaccttccgcaactgggcattgccaatctgcgtgttcgtccagctagcgaattcgctggaaattggcagtcagcaactggcatttacccatcaggaagcgaagcagttttttgattgccgtctgtcatcgccgattgaagctgcagaaagcagtcggatttgtgatgatgtttccggttgggcgacggcactgcagctaatcgccctctccgcccggcagaatactcactcagcccataagtcggcacgccgcctggcgggaatcaatgccagccatctttcggattatctggtcgatgaggttttggataacgtcgatctcgcaacgcgcca

SEQ ID NO: 6 - DNA, 1285 bp, synthetic construct
  misc_feature 1..1285: note = DNA fragment including the PproD promoter, the BBa_BB0032 RBS, the S. flexneri phoN2 gene and the phoN2 transcriptional terminator
  source 1..1285: mol_type = other DNA
cagctaacaccacgtcgtccctatctgctgccctaggtctatgagtggttgctggataactttacgggcatgcataaggctcgtataatatattcagggagaccacaacggtttccctctacaaataattttgtttaacttttactagagtcacacaggaaagtactagatgaaaaccaaaaactttcttcttttttgtattgctacaaatatgatttttatcccctcagcaaatgctctgaaggcagaaggttttctcactcaacaaacttcaccagacagtttgtcaatacttccgccgcctccggcagaggattcagtagtatttctggctgacaaagctcattatgaattcggccgctcgctccgggatgctaatcgtgtacgtctcgctagcgaagatgcatactacgagaattttggtcttgcattttcagatgcttatggcatggatatttcaagggaaaataccccaatcttatatcagttgttaacacaagtactacaggatagccatgattacgccgtgcgtaacgccaaagaatattataaaagagttcgtccattcgttatttataaagacgcaacctgtacacctgataaagatgagaaaatggctatcactggctcttatccctctggtcatgcatcctttggttgggcagtagcactgatacttgcggagattaatcctcaacgtaaagcggaaatacttcgacgtggatatgagtttggagaaagtcgggtcatctgcggtgcgcattggcaaagcgatgtagaggctgggcgtttaatgggagcatcggttgttgcagtacttcataatacacctgaatttaccaaaagccttagcgaagccaaaaaagagtttgaagaattaaatactcctaccaatgaactgaccccataaagctggacagcctgtatcaggctatggagggcccatagacaaatctaccctatatgagcataggaggagtctatgggcacaccacgttttacccctgaatttaagggattactggaaaggctgggacatatccctccggcagaagcagaaaaagcttattatgctgccatcggaaacgatgatctggcaacctgagttcacagataaaacattctctaggaaactcggggcggttccgttcaccacatgcaatgtggtgttgcaggggaacggtctgcccatcccctatgtcgatcaatataacagaaatgacaacttcagattcagggcacaacctaaatatattttaggtcacctctcaaatcgtttgcctga

SEQ ID NO: 7 - DNA, 995 bp, synthetic construct
  misc_feature 1..995: note = DNA fragment including the E. coli cat gene flanked by the FRT sequences
  source 1..995: mol_type = other DNA
gaagttcctattctctagaaagtataggaacttcggcgcgcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaggcgcgccatttaaatgaagttcctattccgaagttcctattctctagaaagtataggaacttcgaagcagctccagcctacacaatgaattc

SEQ ID NO: 8 - DNA, 1158 bp, synthetic construct
  misc_feature 1..1158: note = cDNA encoding chicken ovalbumin
  source 1..1158: mol_type = other DNA
atgggctccatcggtgcagcaagcatggaattttgttttgatgtattcaaggagctcaaagtccaccatgccaatgagaacatcttctactgccccattgccatcatgtcagctctagccatggtatacctgggtgcaaaagacagcaccaggacacaaataaataaggttgttcgctttgataaacttccaggattcggagacagtattgaagctcagtgtggcacatctgtaaacgttcactcttcacttagagacatcctcaaccaaatcaccaaaccaaatgatgtttattcgttcagccttgccagtagactttatgctgaagagagatacccaatcctgccagaatacttgcagtgtgtgaaggaactgtatagaggaggcttggaacctatcaactttcaaacagctgcagatcaagccagagagctcatcaattcctgggtagaaagtcagacaaatggaattatcagaaatgtccttcagccaagctccgtggattctcaaactgcaatggttctggttaatgccattgtcttcaaaggactgtgggagaaagcatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttcccct

SEQ ID NO: 9 - protein, 386 aa, synthetic construct
  REGION 1..386: note = chicken ovalbumin
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP